(19) United States
(12) Patent Application Publication
(10) Pub. No.: US 2023/0338555 A1
(43) Pub. Date: Oct. 26, 2023

(54) CONJUGATED OLIGONUCLEOTIDES AND USES THEREOF

(71) Applicant: Ionis Pharmaceuticals, Inc., Carlsbad, CA (US)

(72) Inventors: Thazha P. Prakash, Carlsbad, CA (US); Mehran Nikan, Oceanside, CA (US); Michael Tanowitz, Solana Beach, CA (US); Chrissa A. Dwyer, San Diego, CA (US); Frank Rigo, Carlsbad, CA (US); Punit P. Seth, Carlsbad, CA (US)

(73) Assignee: Ionis Pharmaceuticals, Inc., Carlsbad, CA (US)

(21) Appl. No.: 17/998,141

(22) PCT Filed: May 18, 2021
(86) PCT No.: PCT/US2021/032906

§ 371 Date: Nov. 7, 2022

Related U.S. Application Data
(60) Provisional application No. 63/026,648, filed on May 18, 2020.

(51) Int. Cl. A61K 47/64; C07K 7/08

(52) U.S. Cl. CPC A61K 47/64; C07K 7/083

(57) ABSTRACT

Disclosed herein are compounds that comprise an oligonucleotide, a conjugate linker, and a conjugate moiety capable of interacting with a cell surface moiety, wherein the oligonucleotide and the conjugate moiety are connected via the conjugate linker. The conjugate moiety may comprise a cell-targeting moiety and a peptide extender. In general, peptide extenders have sufficient length and/or structure to reduce or prevent interaction between the oligonucleotide and the cell-targeting moiety. Such compounds are useful to treat, prevent or ameliorate a condition or disease in an individual with limited off-target effects.

### SEQUENCE LISTING

The present application is being filed along with a Sequence Listing in electronic format. The Sequence Listing is provided as a file entitled CORE0153WOSEQ_ST25.txt created May 14, 2021, which is 90 kb in size. The information in the electronic format of the sequence listing is incorporated herein by reference in its entirety.

### FIELD

The present embodiments provide compounds and methods for targeting cells of interest with an oligonucleotide.

### BACKGROUND

Oligomeric compounds, such as siRNA and single-stranded antisense oligonucleotides (ASOs), have been shown to be useful for regulating gene expression and have proven to be therapeutically effective. Certain chemical modifications of oligomeric compounds can improve the potency, efficacy, and unwanted side effects of oligomeric compounds, allowing for administration of lower doses, reducing the potential for toxicity, and decreasing the overall cost of therapy. Oligomeric compounds can be modified with a conjugate group, e.g., a ligand for a receptor expressed on a cell of interest, which results in targeting the oligomeric compound to one or more tissues of interest.

Sortilin, also referred to as sortilin receptor, is a membrane-bound glycoprotein that is encoded by the SORT1 gene. Sortilin is a member of the vacuolar protein sorting 10 protein (Vps10p) family of sorting receptors which play a role in intracellular protein trafficking. Sortilin functions in protein transport between the Golgi apparatus, endosome, lysosome, and plasma membrane. Sortilin is highly expressed in the central nervous system relative to other tissues. Sortilin includes an N-terminal pro-peptide that prevents premature ligand binding during its synthesis. The pro-peptide is ultimately cleaved from sortilin to produce a functional glycoprotein capable of binding ligands. Neurotensin (NT) is a 13-amino acid endogenous peptide that has a high affinity ligand for sortilin. The receptor-binding site of NT is located on the C-terminal and is comprised of a hexapeptide unit of RRPYIL.

### SUMMARY

Embodiments provided herein are directed to oligomeric compounds that comprise an oligonucleotide and a conjugate group. In certain embodiments, the conjugate group comprises a cell-targeting moiety. In certain embodiments, the conjugate group comprises a conjugate linker that links the cell-targeting moiety to the oligonucleotide. In certain embodiments, the cell-targeting moiety comprises or consists of a cell-targeting peptide. In certain embodiments, the cell-targeting moiety comprises a cell-targeting peptide and a peptide extender that extends from the cell-targeting peptide via an amide bond. In certain embodiments, the conjugate linker links the oligonucleotide to the peptide extender of the cell-targeting moiety. FIGS. 1 and 2A-2D provide representations of exemplary oligomeric compounds for clarity. In general, peptide extenders have sufficient length and/or structure to separate the oligonucleotide and the cell-targeting peptide such that the cell-targeting peptide does not inhibit an activity of the oligonucleotide (e.g., antisense activity) and the oligonucleotide does not inhibit an activity of the cell-targeting peptide (e.g., binding a cell surface moiety).

### BRIEF DESCRIPTION OF THE DRAWINGS

FIG. 1 shows a representative oligomeric compound comprising an oligonucleotide, a conjugate linker, a peptide extender and a cell-targeting peptide.

FIG. 2A shows a representative oligomeric compound wherein an amino terminus of a cell-targeting peptide is covalently connected to a carboxy terminus of a peptide extender, the peptide extender amino terminus is covalently linked to a conjugate linker, and the conjugate linker is covalently connected to the 3′ end of an oligonucleotide.

FIG. 2B shows a representative oligomeric compound wherein a carboxy terminus of a cell-targeting peptide is covalently connected to an amino terminus of a peptide extender, the peptide extender carboxy terminus is covalently linked to a conjugate linker, and the conjugate linker is covalently connected to the 3′ end of an oligonucleotide.

FIG. 2C shows a representative oligomeric compound wherein a carboxy terminus of a cell-targeting peptide is covalently connected to an amino terminus of a peptide extender, the peptide extender carboxy terminus is covalently linked to a conjugate linker, and the conjugate linker is covalently connected to the 5′ end of an oligonucleotide.

FIG. 2D shows a representative oligomeric compound wherein an amino terminus of a cell-targeting peptide is covalently connected to a carboxy terminus of a peptide extender, the peptide extender amino terminus is covalently linked to a conjugate linker, and the conjugate linker is covalently connected to the 5′ end of an oligonucleotide.

### DETAILED DESCRIPTION

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The section headings used herein are for organizational purposes only and are not to be construed as limiting the subject matter described. All documents, or portions of documents, cited in this application, including, but not limited to, patents, patent applications, articles, books, treatises, and GenBank, NCBI, and ENSEMBL reference sequence records are hereby expressly incorporated by reference for the portions of the document discussed herein, as well as in their entirety.

It is understood that the sequence set forth in each SEQ ID NO of an oligonucleotide in the examples contained herein is independent of any modification to a sugar moiety, an internucleoside linkage, or a nucleobase. As such, oligonucleotides defined by a SEQ ID NO may comprise, independently, one or more modifications to a sugar moiety, an internucleoside linkage, or a nucleobase.

It is understood that throughout the specification, the first letter in a peptide sequence is the first amino acid of the peptide at the N-terminus and the last letter in a peptide sequence is the last amino acid of the peptide at the C-terminus unless indicated otherwise.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings: As used herein, “2′-deoxynucleoside” means a nucleoside comprising a 2′-H(H) deoxyfuranosyl sugar moiety. In certain embodiments, a 2′-deoxynucleoside is a 2′-β-D-deoxynucleoside and comprises a 2′-β-D-deoxyribosyl sugar moiety, which has the β-D configuration as found in naturally occurring deoxyribonucleic acids (DNA). In certain embodiments, a 2′-deoxynucleoside or nucleoside comprising an unmodified 2′-deoxyfuranosyl sugar moiety may comprise a modified nucleobase or may comprise an RNA nucleobase (uracil).

As used herein, “2′-MOE” or “2′-MOE sugar moiety” means a 2′—OCH₂CH₂OCH₃ group in place of the 2′—OH group of a ribosyl sugar moiety. “MOE” means methoxyethyl.

As used herein, “2′-MOE nucleoside” means a nucleoside comprising a 2′-MOE sugar moiety.

As used herein, “2′-OMe” or “2′-O-methyl sugar moiety” means a 2′—OCH₃ group in place of the 2′-OH group of a ribosyl sugar moiety.

As used herein, “2′-OMe nucleoside” means a nucleoside comprising a 2′-OMe sugar moiety. As used herein, “2′-NMA” means a —O—CH₂—C(═O)—NH—CH₃ group in place of the 2′—OH group of a ribosyl sugar moiety. A “2′-NMA sugar moiety” is a sugar moiety with a 2′-O—CH₂—C(═O)—NH—CH₃ group in place of the 2′—OH group of a ribosyl sugar moiety. Unless otherwise indicated, a 2′-NMA sugar moiety is in the β-D configuration. “NMA” means O—N-methyl acetamide.

As used herein, “2′-NMA nucleoside” means a nucleoside comprising a 2′-NMA sugar moiety.

As used herein, “2′-substituted nucleoside” means a nucleoside comprising a 2′-substituted sugar moiety. As used herein, “2′-substituted” in reference to a sugar moiety means a sugar moiety comprising at least one 2′-substituent group other than H or OH.

As used herein, “5-methyl cytosine” means a cytosine modified with a methyl group attached to the 5 position. A 5-methyl cytosine is a modified nucleobase.

As used herein, “about” means plus or minus 7% of the provided value.

As used herein, “administering” means providing a pharmaceutical agent to an animal.

As used herein, “animal” means a human or non-human animal. In certain embodiments, the animal is a human.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound.

As used herein, “antisense compound” means an oligomeric compound capable of achieving at least one antisense activity.

As used herein, “bicyclic nucleoside” or “BNA” means a nucleoside comprising a bicyclic sugar moiety.

As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

As used herein, “cell-targeting moiety” means a group of atoms that is capable of interacting with a cell-surface receptor or a cell-surface moiety. In certain embodiments, a cell-targeting moiety is capable of binding the cell-surface receptor or the cell-surface moiety. In certain embodiments, a cell-targeting moiety is capable of being internalized when it interacts with or binds the cell-surface receptor or the cell-surface moiety. In certain embodiments, a cell-targeting moiety comprises a cell-targeting peptide. In certain embodiments, a cell-targeting moiety consists of a cell-targeting peptide.

As used herein, “cell-targeting peptide” means a peptide that is capable of interacting with a cell-surface receptor or a cell-surface moiety. In certain embodiments, a cell-targeting peptide is capable of binding the cell-surface receptor or the cell-surface moiety. In certain embodiments, a cell-targeting peptide is capable of being internalized when it interacts with or binds the cell-surface receptor or the cell-surface moiety.

As used herein, “cell-surface moiety” means a moiety present on the surface of a cell that is available to interact with matter external to the cell. In certain embodiments, a portion of the cell-surface moiety is integral with the cell membrane of the cell. Non-limiting examples of cell-surface moieties are lipids, proteins, and carbohydrates. In certain embodiments, a cell-surface moiety is a cell-surface receptor.

As used herein, “cell-surface receptor” means a protein receptor expressed on the surface of a cell that is available to interact with a corresponding ligand. The ligand may be endogenous or exogenous.

As used herein, “cleavable moiety” means a bond or group of atoms that is cleaved under physiological conditions, for example, inside a cell or an animal.

As used herein, “complementary” in reference to an oligonucleotide means that at least 70% of the nucleobases of the oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. The term, “complementary nucleobases,” means nucleobases that are capable of forming hydrogen bonds with one another. Complementary nucleobase pairs include adenine (A) and thymine (T), adenine (A) and uracil (U), cytosine (C) and guanine (G), 5-methyl cytosine (mC) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that oligonucleotides are complementary to another oligonucleotide or nucleic acid at each nucleoside of the oligonucleotide.

As used herein, “conjugate group” means a group of atoms that is directly attached to an oligonucleotide. In certain embodiments, a conjugate group comprises a conjugate moiety and a conjugate linker that attaches the conjugate moiety to the oligonucleotide.

As used herein, “conjugate linker” means a single bond or group of atoms comprising at least one bond that connects a conjugate moiety to an oligonucleotide. In certain embodiments, a conjugate linker comprises a cleavable moiety.

As used herein, “conjugate moiety” means a group of atoms that is attached to an oligonucleotide via a conjugate linker. In certain embodiments, a conjugate moiety comprises a cell-targeting moiety. In certain embodiments, a cell-targeting moiety comprises or consists of a cell-targeting peptide. In certain embodiments, a cell-targeting moiety comprises or consists of peptide extender and a cell-targeting peptide.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are oligomeric compounds disclosed herein. In certain embodiments, the oligomeric compounds are antisense compounds. In certain embodiments, the molecules are modified oligonucleotides. In certain embodiments, the molecules are oligomeric compounds comprising modified oligonucleotides.

As used herein, “gapmer” means a modified oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.” Unless otherwise indicated, “gapmer” refers to a sugar motif. Unless otherwise indicated, the sugar moiety of each nucleoside of the gap is a 2′-β-D-deoxyribosyl sugar moiety. Thus, by way of example, the term “MOE gapmer” indicates a gapmer having a gap comprising 2′-β-D-deoxynucleosides and wings comprising 2′-MOE nucleosides. Unless otherwise indicated, a MOE gapmer may comprise one or more modified internucleoside linkages and/or modified nucleobases and such modifications do not necessarily follow the gapmer pattern of the sugar modifications.

As used herein, “human peptide” means a peptide encoded by a human gene, as described in a published database such as NCBI GenBank®, GeneCards®, or Ensembl Genome Browser.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, “% identical,” with regards to an amino acid sequence, means the percentage of amino acids that are identical between two amino acid sequences when the amino acid sequences are aligned for maximal similarity.

As used herein, the term “internucleoside linkage” is the covalent linkage between adjacent nucleosides in an oligonucleotide. As used herein “modified internucleoside linkage” means any internucleoside linkage other than a phosphodiester internucleoside linkage. “Phosphorothioate internucleoside linkage” is a modified internucleoside linkage in which one of the non-bridging oxygen atoms of a phosphodiester internucleoside linkage is replaced with a sulfur atom.

As used herein, “non-bicyclic modified sugar moiety” means a modified sugar moiety that comprises a modification, such as a substituent, that does not form a bridge between two atoms of the sugar to form a second ring.

As used herein, “mismatch” or “non-complementary” means a nucleobase of a first oligonucleotide that is not complementary with the corresponding nucleobase of a second oligonucleotide or target nucleic acid when the first and second oligonucleotide are aligned.

As used herein, “motif” means the pattern of unmodified and/or modified sugar moieties, nucleobases, and/or internucleoside linkages, in an oligonucleotide.

As used herein, “nucleobase” means an unmodified nucleobase or a modified nucleobase. As used herein an “unmodified nucleobase” is adenine (A), thymine (T), cytosine (C), uracil (U), and guanine (G). As used herein, a “modified nucleobase” is a group of atoms other than unmodified A, T, C, U, or G capable of pairing with at least one unmodified nucleobase. A “5-methyl cytosine” is a modified nucleobase. A universal base is a modified nucleobase that can pair with any one of the five unmodified nucleobases. As used herein, “nucleobase sequence” means the order of contiguous nucleobases in a nucleic acid or oligonucleotide independent of any sugar or internucleoside linkage modification.

As used herein, “nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. “Linked nucleosides” are nucleosides that are connected in a contiguous sequence (i.e., no additional nucleosides are presented between those that are linked).

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound. The term “oligomeric duplex” means a duplex formed by two oligomeric compounds having complementary nucleobase sequences. Each oligomeric compound of an oligomeric duplex may be referred to as a “duplexed oligomeric compound.”

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “peptide extender” means a peptide that extends from a cell-targeting moiety via an amide bond and attaches to an oligonucleotide via a conjugate linker. In certain embodiments, the cell-targeting moiety comprises or consists of a cell-targeting peptide, and the peptide extender extends from the cell-targeting peptide.

As used herein, “pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by an animal. In certain embodiments, a pharmaceutically acceptable carrier or diluent is sterile water, distilled water for injection, sterile saline, sterile buffer solution or sterile artificial cerebrospinal fluid.

As used herein “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein “pharmaceutical composition” means a mixture of substances suitable for administering to an animal. For example, a pharmaceutical composition may comprise an oligomeric compound and a sterile aqueous solution. In certain embodiments, a pharmaceutical composition shows activity in free uptake assay in certain cell lines.

As used herein, “phosphoramidite morpholino oligomer” means a nonionic antisense oligonucleotide, wherein each nucleoside comprises a morpholino sugar surrogate and each internucleoside linkage is a phosphoramidite linkage. One nucleotide of a phosphoramidite morpholino oligomer is shown below:

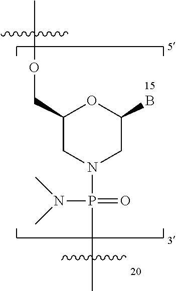

In certain embodiments, a phosphoramidite morpholino oligomer is capable of modifying splicing of a target nucleic acid.

As used herein, “reducing or inhibiting the amount or activity” refers to a reduction or blockade of the transcriptional expression or activity relative to the transcriptional expression or activity in an untreated or control sample and does not necessarily indicate a total elimination of transcriptional expression or activity.

As used herein, “RNAi compound” means an antisense compound that acts, at least in part, through RISC or Ago2 to modulate a target nucleic acid and/or protein encoded by a target nucleic acid. RNAi compounds include, but are not limited to double-stranded siRNA, single-stranded RNA (ssRNA), and microRNA, including microRNA mimics. In certain embodiments, an RNAi compound modulates the amount, activity, and/or splicing of a target nucleic acid. The term RNAi compound excludes antisense compounds that act through RNase H.

As used herein, “RNase H compound” means an antisense compound that acts through RNase H to modulate a target nucleic acid and/or protein encoded by a target nucleic acid. In certain embodiments, RNase H compounds are single-stranded. In certain embodiments, RNase H compounds are double-stranded. RNase H compounds may comprise conjugate groups and/or terminal groups. In certain embodiments, an RNase H compound modulates the amount and/or activity of a target nucleic acid. The term RNase H compound excludes antisense compounds that act principally through RISC/Ago2.

As used herein, “self-complementary” in reference to an oligonucleotide means an oligonucleotide that at least partially hybridizes to itself.

As used herein, “stabilized phosphate group” refers to a 5′-chemical moiety that results in stabilization of a 5′-phosphate moiety of the 5′-terminal nucleoside of an oligonucleotide, relative to the stability of an unmodified 5′-phosphate of an unmodified nucleoside under biologic conditions. Such stabilization of a 5′-phophate group includes but is not limited to resistance to removal by phosphatases. Stabilized phosphate groups include, but are not limited to, 5′-vinyl phosphonates and 5′-cyclopropyl phosphonate.

As used herein, “stereorandom chiral center” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the results of a synthetic method that is not designed to control the stereochemical configuration. In certain embodiments, a stereorandom chiral center is a stereorandom phosphorothioate internucleoside linkage.

As used herein, “standard cell assay” means assay(s) described in the Examples and reasonable variations thereof.

As used herein, “subject” refers to a human or non-human animal, including, but not limited to, mice, rats, rabbits, dogs, cats, pigs, and non-human primates, including, but not limited to, monkeys and chimpanzees.

As used herein, “sugar moiety” means an unmodified sugar moiety or a modified sugar moiety. As used herein, “unmodified sugar moiety” means a 2′-OH(H) ribosyl moiety, as found in RNA (an “unmodified RNA sugar moiety”), or a 2′-H(H) deoxyribosyl moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate.

As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

As used herein, “target nucleic acid” and “target RNA” mean a nucleic acid that an antisense compound is designed to affect.

As used herein, “terminal group” means a chemical group or group of atoms that is covalently linked to a terminus of an oligonucleotide.

As used herein, a “neutral amino acid” is an amino acid having a side chain that does not comprise a positive charge or a negative charge when in solution at pH=7.0. Neutral amino acids include, but are not limited to, glycine, alanine, leucine, isoleucine, valine, serine, cysteine, methionine, proline, threonine, tyrosine, phenylalanine, tryptophan, beta-alanine, and 2-aminoisobutyric acid.

As used herein, a “charged amino acid” is an amino acid having a side chain that comprises a positive charge or a negative charge when in solution at pH=7.0. A “basic amino acid” has a side chain that comprises a positive charge when in solution at pH=7.0. Basic amino acids include, but are not limited to, lysine, arginine, and ornithine. A “acidic amino acid” has a side chain that comprises a negative charge when in solution at pH=7.0. Acidic amino acids include, but are not limited to, glutamic acid and aspartic acid.

As used herein, an “aliphatic amino acid” is an amino acid having a side chain composed of H and C. Aliphatic amino acids include, but are not limited to, glycine, alanine, leucine, isoleucine, valine, beta-alanine, 2-aminoisobutyric acid.

As used herein, an “aromatic amino acid” is an amino acid having an aromatic ring in its side chain. Aromatic amino acids include, but are not limited to, phenylalanine, tyrosine, and tryptophan.

As used herein, a “cyclic amino acid” is an amino acid where the side chain connects to the backbone amide to form a cyclic structure. “Cyclic amino acid” includes, but is not limited to, proline or hydroxyproline.

As used herein, a “compact amino acid” is an amino acid having a side chain with a molecular weight less than 50 g/mol. “Compact amino acids” include, but are not limited to, glycine, alanine, proline, cysteine, serine, and threonine.

As used herein, “side chain” means a sub-structure of an amino acid attached to the alpha carbon of the amino acid.

As used herein, “reactive group” means a sub-structure of an amino acid that can form bonds with another compound, e.g., another sub-structure of another amino acid, amine or carboxylic acid.

### CERTAIN EMBODIMENTS

The present disclosure provides the following non-limiting numbered embodiments:
Embodiment 1. An oligomeric compound comprising an oligonucleotide; and a conjugate group comprising a conjugate moiety, and a conjugate linker that links the conjugate moiety to the oligonucleotide, wherein the conjugate moiety comprises a cell-targeting peptide that binds human sortilin.
Embodiment 2. The oligomeric compound of embodiment 1, wherein the cell-targeting peptide comprises or consists of a human neurotensin peptide or a sortilin-binding portion thereof.
Embodiment 3. The oligomeric compound of embodiment 2, wherein the amino acid sequence of the human neurotensin peptide comprises or consists of an amino acid sequence selected from XLYENKPRRPYIL (SEQ ID NO: 3) LYENKPRRPYIL (SEQ ID NO: 32), ELYENKPRRPYIL (SEQ ID NO: 72), XLYENKPKRPYIL (SEQ ID NO: 73), XLYENKPRKPYIL (SEQ ID NO: 74), and XLYENKPKKPYIL (SEQ ID NO: 75), wherein X is lysine or 2-azido-acetyl lysine.
Embodiment 4. The oligomeric compound of embodiment 2, wherein the amino acid sequence of the human neurotensin peptide sequence is at least 75%, at least 80%, at least 85%, at least 90%, or at least 95% identical to an amino acid sequence selected from XLYENKPRRPYIL (SEQ ID NO: 3), LYENKPRRPYIL (SEQ ID NO: 32), ELYENKPRRPYIL (SEQ ID NO: 72), XLYENKPKRPYIL (SEQ ID NO: 73), XLYENKPRKPYIL (SEQ ID NO: 74), and XLYENKPKKPYIL (SEQ ID NO: 75), wherein X is lysine or azido-acetyl lysine.
Embodiment 5. The oligomeric compound of embodiment 1, wherein the cell-targeting peptide comprises or consists of a sortilin propeptide or a sortilin-binding portion thereof.
Embodiment 6. The oligomeric compound of embodiment 5, wherein the amino acid sequence of the sortilin propeptide comprises or consists of an amino acid sequence selected from CQDRLDAPPPPAAPLPRWSGPIGVSWGLRAAAAGGAFPRGGRWRR (SEQ ID NO: 76) and CQDRLDAPPPPAAPLPRWSGPIGVSWGLR (SEQ ID NO: 77).
Embodiment 7. The oligomeric compound of embodiment 5, wherein the amino acid sequence of the sortilin propeptide is at least 75%, at least 80%, at least 85%, at least 90%, or at least 95% identical to an amino acid sequence selected from CQDRLDAPPPPAAPLPRWSGPIGVSWGLRAAAAGGAFPRGGRWRR (SEQ ID NO: 76) and CQDRLDAPPPPAAPLPRWSGPIGVSWGLR (SEQ ID NO: 77).
Embodiment 8. The oligomeric compound of any one of embodiments 1-7, wherein the conjugate moiety comprises a peptide extender that extends the cell-targeting peptide.
Embodiment 9. The oligomeric compound of embodiment 8, wherein the amino acid sequence of the peptide extender and cell-targeting peptide together is selected from: XPPPAGSSPGLYENKPRRPYIL (SEQ ID NO: 5), XPAPSGPSPGLYENKPRRPYIL (SEQ ID NO: 6), XAGSIKPPPAGSSPGLYENKPRRPYIL (SEQ ID NO: 7), and XAGMSGASAGLYENKPRRPYIL (SEQ ID NO: 8), wherein X is lysine or 2-azido-acetyl lysine.
Embodiment 10. The oligomeric compound of embodiment 8, wherein the amino acid sequence of the peptide extender and cell-targeting peptide together is at least 75%, at least 80%, at least 85%, at least 90%, or at least 95% identical to an amino acid sequence selected from XPPPAGSSPGLYENKPRRPYIL (SEQ ID NO: 5), XPAPSGPSPGLYENKPRRPYIL (SEQ ID NO: 6), XAGSIKPPPAGSSPGLYENKPRRPYIL (SEQ ID NO: 7), and XAGMSGASAGLYENKPRRPYIL (SEQ ID NO: 8), wherein X is lysine or 2-azido-acetyl lysine.
Embodiment 11. An oligomeric compound comprising an oligonucleotide; and a conjugate group comprising a conjugate moiety and a conjugate linker, wherein the conjugate moiety comprises a cell-targeting peptide and a peptide extender that extends the cell-targeting peptide, wherein the conjugate linker links the conjugate moiety to the oligonucleotide via the peptide extender, wherein the cell-targeting peptide is at least 70%, at least 75%, at least 80%, at least 85%, at least 90%, or at least 95% identical to a peptide encoded by a human gene.
Embodiment 12. The oligomeric compound of embodiment 11, wherein the peptide extender is less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, less than about 5% identical to a human peptide.
Embodiment 13. The oligomeric compound of embodiment 11, wherein the peptide extender does not comprise a human peptide.
Embodiment 14. The oligomeric compound of any one of embodiments 8-13, wherein the peptide extender comprises or consists essentially of 3 to 50, 3 to 45, 3 to 40, 3 to 35, 3 to 30, 3 to 25, 3 to 20, 3 to 15, 3 to 10, 6 to 50, 6 to 45, 6 to 40, 6 to 35, 6 to 30, 6 to 25, 6 to 20, 6 to 15, or 6 to 10 amino acids.
Embodiment 15. The oligomeric compound of any one of embodiments 8-14, wherein the peptide extender comprises at least 6, at least 7, at least 8, at least 9, or at least 10 amino acids.
Embodiment 16. The oligomeric compound of any one of embodiments 8-15, wherein the peptide extender comprises at least one amino acid selected from serine, proline, hydroxyproline, methionine, cysteine and tyrosine.
Embodiment 17. The oligomeric compound of embodiment 9, wherein the peptide extender comprises at least 2 or at least 3 amino acids selected from serine, proline, hydroxyproline, methionine, cysteine and tyrosine.
Embodiment 18. The oligomeric compound of embodiment 17, wherein the at least 2 or at least 3 amino acids are contiguous.
Embodiment 19. The oligomeric compound of any one of embodiments 8-18, wherein the peptide extender comprises three contiguous proline residues.
Embodiment 20. The oligomeric compound of any one of embodiments 8-19, wherein the peptide extender has a molecular weight of about 400 g/mol to about 1800 g/mol.
Embodiment 21. The oligomeric compound of any one of embodiments 8-13, wherein the length of the peptide extender is at least 15 Å in a solvent selected from water and phosphate buffered saline.
Embodiment 22. The oligomeric compound of any one of embodiments 8-14, wherein the conjugate linker is connected to the 5′ end of the oligonucleotide.
Embodiment 23. The oligomeric compound of any one of embodiments 8-14, wherein the conjugate linker is connected to the 3′ end of the oligonucleotide.
Embodiment 24. The oligomeric compound of embodiment 22 or 23, wherein the conjugate linker is connected to a carboxy terminus of the peptide extender.
Embodiment 25. The oligomeric compound of embodiment 22 or 23, wherein the conjugate linker is connected to an amino terminus of the peptide extender.
Embodiment 26. The oligomeric compound of any one of embodiments 8-25, wherein the peptide extender has an amino acid sequence selected from: KPPPAGSSPG (SEQ ID NO: 48), X₁PPPAGSSPG (SEQ ID NO: 49), X₁X₂PPAGSSPG (SEQ ID NO: 50), X₁P X₂PAGSSPG (SEQ ID NO: 51), X₁PP X₂AGSSPG (SEQ ID NO: 52), X₁PPP X₂GSSPG (SEQ ID NO: 53), X₁PPPA X₂SSPG (SEQ ID NO: 54), X₁PPPAG X₂SPG (SEQ ID NO: 55), X₁PPPAGS X₂PG (SEQ ID NO: 56), X₁PPPAGSS X₂G (SEQ ID NO: 57), and X₁PPPAGSSPX (SEQ ID NO: 58), wherein X₁ is selected from lysine, 2-azido-acetyl lysine, D-lysine, L-lysine, N6-(2-azido-acetyl)-D-lysine, and N⁶-(2-azido-acetyl)-L-lysine; and X₂ is any amino acid.
Embodiment 27. The oligomeric compound of any one of embodiments 8-25, wherein the peptide extender has an amino acid sequence that is at least 70%, at least 75%, at least 80%, at least 85%, at least 90%, at least 95%, or 100% identical to the amino acid sequence of KPPPAGSSPG (SEQ ID NO: 48).
Embodiment 28. The oligomeric compound of any one of embodiments 8-25, wherein the peptide extender has an amino acid sequence selected from: CPPPAGSSPG (SEQ ID NO: 59), XPPPAGSSPG (SEQ ID NO: 49), CXPPAGSSPG (SEQ ID NO: 60), CPXPAGSSPG (SEQ ID NO: 61), CPPXAGSSPG (SEQ ID NO: 62), CPPPXGSSPG (SEQ ID NO: 63), CPPPAXSSPG (SEQ ID NO: 64), CPPPAGXSPG (SEQ ID NO: 65), CPPPAGSXPG (SEQ ID NO: 66), CPPPAGSSXG (SEQ ID NO: 67), and CPPPAGSSPX (SEQ ID NO: 68), wherein X is any amino acid.
Embodiment 29. The oligomeric compound of any one of embodiments 8-25, wherein the peptide extender comprises an amino acid sequence that is at least 70%, at least 75%, at least 80%, at least 85%, at least 90%, at least 95%, or 100% identical to the amino acid sequence of CPPPAGSSPG (SEQ ID NO: 59).
Embodiment 30. The oligomeric compound of any one of embodiments 1-18, wherein the peptide extender is represented by amino acid sequence CAGSIKPPPAGSSPG (SEQ ID NO: 70) or KAGSIKPPPAGSSPG (SEQ ID NO: 71).
Embodiment 31. The oligomeric compound of any one of embodiments 8-25, wherein the peptide extender has an amino acid sequence selected from: XPAPSGPSPG (SEQ ID NO: 81), XAGSIKPPPAGSSPG (SEQ ID NO: 82), and XAGMSGASAG (SEQ ID NO: 83), wherein X is selected from lysine, cysteine, and N⁶-(2-azido-acetyl)-lysine.
Embodiment 32. The oligomeric compound of any one of embodiments 26, 27 and 31, wherein the lysine of the peptide extender is D-Lysine.
Embodiment 33. The oligomeric compound of any one of embodiments 8-32, wherein the peptide extender has a net charge of 0, 1, or 2 at neutral pH.
Embodiment 34. The oligomeric compound of any one of embodiments 8-25, wherein the peptide extender comprises a structure of the formula X_{L}—(X_{S})ₙ—X₁—X₂—X₃—X₄—X₅—X₆—X₇—X₈—X₉, wherein: X_{L} comprises a linker amino acid; n is from 0 to 5; at least one of X_{S} is independently selected from an aliphatic amino acid, lysine, and serine; at least one of X₁—X₉ is a neutral amino acid.
Embodiment 35. The oligomeric compound of embodiment 34, wherein each of X₁—X₉ is a neutral amino acid.
Embodiment 36. The oligomeric compound of embodiment 34 or 35, wherein the side chain of X_{L} comprises a reactive group.
Embodiment 37. The oligomeric compound of embodiment 36, wherein the side chain of X L comprises a nucleophile.
Embodiment 38. The oligomeric compound of embodiment 36, wherein the side chain of X L comprises an electrophile.
Embodiment 39. The oligomeric compound of embodiment 36, wherein the side chain of X_{L} comprises an amine or a sulfhydryl.
Embodiment 40. The oligomeric compound of embodiment 39, wherein X_{L} is lysine.
Embodiment 41. The oligomeric compound of embodiment 39, wherein X_{L} is cysteine.
Embodiment 42. The oligomeric compound of embodiment 34, wherein n is 0.
Embodiment 43. The oligomeric compound of any one of embodiments 34-41, wherein n is 4.
Embodiment 44. The oligomeric compound of embodiment 43, wherein (X_{S})₄ is Ala-Gly-Ser-Ile.
Embodiment 45. The oligomeric compound of embodiment 43, wherein each amino acid of (X_{S})₄ is independently selected from an aliphatic amino acid, lysine, and serine.
Embodiment 46. The oligomeric compound of embodiment 43, wherein at least one amino acid of (X_{S})₄ is not lysine.
Embodiment 47. The oligomeric compound of embodiment 43, wherein at least one amino acid of (X_{S})₄ is not arginine.
Embodiment 48. The oligomeric compound of embodiment 43, wherein (X_{S})₄ does not comprise a lysine.
Embodiment 49. The oligomeric compound of embodiment 43, wherein (X_{S})₄ does not comprise an arginine.
Embodiment 50. The oligomeric compound of any of embodiments 28-42, wherein each of X₁, X₂, and X₃ is selected from alanine, proline, glycine, cysteine, methionine, or tyrosine.
Embodiment 51. The oligomeric compound of embodiment 50, wherein X₁ is alanine or proline.
Embodiment 52. The oligomeric compound of embodiment 51, wherein X₁ is alanine.
Embodiment 53. The oligomeric compound of embodiment 51, wherein X₁ is proline.
Embodiment 54. The oligomeric compound of any of embodiments 50-53, wherein X₂ is proline, alanine, or glycine.
Embodiment 55. The oligomeric compound of embodiment 54, wherein X₂ is proline.
Embodiment 56. The oligomeric compound of embodiment 54, wherein X₂ is alanine.
Embodiment 57. The oligomeric compound of embodiment 54, wherein X₂ is glycine.
Embodiment 58. The oligomeric compound of any of embodiments 34-57, wherein X₃ is proline, serine, or methionine.
Embodiment 59. The oligomeric compound of embodiment 58, wherein X₃ is proline.
Embodiment 60. The oligomeric compound of embodiment 58, wherein X₃ is serine.
Embodiment 61. The oligomeric compound of embodiment 58, wherein X₃ is methionine.
Embodiment 62. The oligomeric compound of any of embodiments 34-61, wherein X₄ is an aliphatic amino acid, serine, or cysteine.
Embodiment 63. The oligomeric compound of any of embodiments 34-61, wherein X₄ is a compact amino acid.
Embodiment 64. The oligomeric compound of embodiment 62 or 63, wherein X₄ is selected from alanine, serine, glycine, or cysteine.
Embodiment 65. The oligomeric compound of any of embodiments 62-64, wherein X₄ is alanine.
Embodiment 66. The oligomeric compound of any of embodiments 62-64, wherein X₄ is serine.
Embodiment 67. The oligomeric compound of any of embodiments 34-66, wherein X₅ is glycine.
Embodiment 68. The oligomeric compound of any of embodiments 34-67, wherein X₆ is a compact amino acid.
Embodiment 69. The oligomeric compound of any of embodiments 34-67, wherein X₆ is selected from alanine, proline, serine, glycine, cysteine, methionine, or tyrosine.
Embodiment 70. The oligomeric compound of embodiment 68 or 69, wherein X₆ is serine.
Embodiment 71. The oligomeric compound of embodiment 68 or 69, wherein X₆ is proline.
Embodiment 72. The oligomeric compound of embodiment 68 or 69, wherein X₆ is alanine.
Embodiment 73. The oligomeric compound of any of embodiments 34-72, wherein X₇ is a compact amino acid.
Embodiment 74. The oligomeric compound of embodiment 72, wherein X₇ is serine.
Embodiment 75. The oligomeric compound of any of embodiments 34-74, wherein X₈ is a compact amino acid.
Embodiment 76. The oligomeric compound of any of embodiments 34-74, wherein X₈ is selected from alanine, proline, glycine, cysteine, methionine, or tyrosine.
Embodiment 77. The oligomeric compound of embodiment 75 or 76, wherein X₈ is proline.
Embodiment 78. The oligomeric compound of embodiment 75 or 76, wherein X₈ is alanine.
Embodiment 79. The oligomeric compound of any of embodiments 34-78, wherein X₉ is a compact amino acid.
Embodiment 80. The oligomeric compound of embodiment 79, wherein X₉ is glycine.
Embodiment 81. The oligomeric compound of any of embodiments 34-80, wherein the peptide extender comprises at least two cyclic amino acids.
Embodiment 82. The oligomeric compound of any of embodiments 34-81, wherein the peptide extender comprises at least three cyclic amino acids.
Embodiment 83. The oligomeric compound of embodiment 81 or 82, wherein the peptide extender comprises at least two prolines.
Embodiment 84. The oligomeric compound of embodiment 81 or 82, wherein the peptide extender comprises at least three prolines.
Embodiment 85. The oligomeric compound of embodiment 81 or 82, wherein the peptide extender comprises exactly three prolines.
Embodiment 86. The oligomeric compound of any of embodiments 34-85, wherein the peptide extender comprises at least five, at least six, at least seven, at least eight or at least nine compact amino acids.
Embodiment 87. The oligomeric compound of any of embodiments 34-85, wherein each of X₄, X₅, X₆, X₇, X₈, and X₉ are compact amino acids.
Embodiment 88. The oligomeric compound of any of embodiments 34-85, wherein at least five of X₄, X₅, X₆, X₇, X₈, and X₉ are compact amino acids.
Embodiment 89. The oligomeric compound of any of embodiments 34-85, wherein the peptide extender comprises at least two or at least three glycines.
Embodiment 90. The oligomeric compound of any of embodiments 34-85, wherein the peptide extender does not comprise more than two or more than three glycines.
Embodiment 91. The oligomeric compound of any of embodiments 34-90, wherein the peptide extender comprises at least two serines.
Embodiment 92. The oligomeric compound of any of embodiments 34-90, wherein the peptide extender comprises not more than two or more than three serines.
Embodiment 93. The oligomeric compound of any of embodiments 34-92, wherein the peptide extender does not comprise the sequence GSSG.
Embodiment 94. The oligomeric compound of any of embodiments 34-93, wherein the peptide extender does not comprise the sequence GSSS.
Embodiment 95. The oligomeric compound of any of embodiments 34-94, wherein the peptide extender does not comprise the sequence SSYG.
Embodiment 96. The oligomeric compound of any of embodiments 34-95, wherein the peptide extender comprises at least three different amino acid types selected from acidic, basic, aliphatic, cyclic, or aromatic.
Embodiment 97. The oligomeric compound of any of embodiments 34-96, wherein the peptide extender comprises at least four different amino acid types selected from acidic, basic, aliphatic, cyclic, or aromatic.
Embodiment 98. The oligomeric compound of any of embodiments 34-97, wherein the peptide extender comprises at least five different amino acid types selected from acidic, basic, aliphatic, cyclic, or aromatic.
Embodiment 99. The oligomeric compound of any of embodiments 34-98, wherein the peptide extender does not have a motif of ABAB, wherein A is an amino acid of one type and B is an amino acid of another type.
Embodiment 100. The oligomeric compound of any of embodiments 34-99, wherein the peptide extender does not have more than three consecutive amino acids of the same type.
Embodiment 101. The oligomeric compound of any of embodiments 34-100, wherein X₅ and X₉ are glycine.
Embodiment 102. The oligomeric compound of any of embodiments 34-100, wherein X₅ and X₉ are glycine and X₇ is serine.
Embodiment 103. The oligomeric compound of any of embodiments 34-100, wherein X₉ is linked to another amino acid by a peptide bond.
Embodiment 104. The oligomeric compound of any one of embodiments 1-8 and 11-103, wherein the carboxy terminus of the peptide extender is connected to the amino terminus of the cell-targeting peptide.
Embodiment 105. The oligomeric compound of any one of embodiments 1-8 and 11-103, wherein an amino terminus of the peptide extender is connected to a carboxy terminus of the peptide.
Embodiment 106. The oligomeric compound of any one of embodiments 1-105, wherein the conjugate linker comprises maleimide.
Embodiment 107. The oligomeric compound of any one of embodiments 1-105, wherein the conjugate linker and the oligonucleotide are connected via click chemistry.
Embodiment 108. The oligomeric compound of any one of embodiments 1-107, wherein the oligonucleotide is a single stranded oligonucleotide.
Embodiment 109. The oligomeric compound of any one of embodiments 1-107, wherein the oligonucleotide is an antisense strand and the oligomeric compound comprises a sense strand that forms a duplex with the antisense strand, or wherein the oligonucleotide is a sense strand and the oligomeric compound comprises an antisense strand that forms a duplex with the sense strand.
Embodiment 110. The oligomeric compound of any one of embodiments 1-109, wherein the oligonucleotide comprises at least one deoxyribonucleoside.
Embodiment 111. The oligomeric compound of any one of embodiments 1-110, wherein the oligonucleotide comprises at least one ribonucleoside.
Embodiment 112. The oligomeric compound of any one of embodiments 1-111, wherein the oligonucleotide comprises 8 linked nucleosides to 50 linked nucleosides.
Embodiment 113. The oligomeric compound of any one of embodiments 1-112, wherein the oligonucleotide is a modified oligonucleotide comprising at least one of a modified internucleoside linkage, a modified sugar moiety, and a modified nucleobase.
Embodiment 114. The oligomeric compound of embodiment 113, wherein the modified sugar moiety is a non-bicyclic sugar moiety.
Embodiment 115. The oligomeric compound of embodiment 114, wherein the non-bicyclic sugar moiety comprises a 2′-MOE sugar moiety or 2′-OMe sugar moiety.
Embodiment 116. The oligomeric compound of embodiment 113, wherein the modified sugar moiety is a bicyclic sugar moiety.
Embodiment 117. The oligomeric compound of embodiment 116, wherein the bicyclic sugar moiety comprises a 2′-4′ bridge selected from —O—CH₂—; and —O—CH(CH₃)—.
Embodiment 118. The oligomeric compound of any one of embodiments 108-117, wherein the oligonucleotide is a gapmer.
Embodiment 119. The oligomeric compound of any one of embodiments 108 or 112-113, wherein the oligonucleotide is a phosphoramidite morpholino oligomer.
Embodiment 120. The oligomeric compound of any one of embodiments 1-119, wherein the peptide extender enhances uptake of the oligonucleotide into a cell relative to a control oligomeric compound without the peptide extender, wherein the oligonucleotide and the conjugate moiety of the control oligomeric compound are linked only by the conjugate linker.
Embodiment 121. An oligomeric duplex comprising an oligomeric compound of any of embodiments 1-120.
Embodiment 122. An antisense compound comprising or consisting of an oligomeric compound of any of embodiments 1-120 or an oligomeric duplex of embodiment 121.
Embodiment 123. A pharmaceutical composition comprising the oligomeric compound of any one of embodiments 1-120, the oligomeric duplex of embodiment 121, or the antisense compound of embodiment 122, and a pharmaceutically acceptable carrier or diluent.
Embodiment 124. The pharmaceutical composition of embodiment 123, wherein the pharmaceutically acceptable carrier or diluent comprises phosphate buffered saline.
Embodiment 125. A method comprising administering the oligomeric compound of any one of embodiments 1-120, the oligomeric duplex of embodiment 121, the antisense compound of embodiment 122, or the pharmaceutical composition of embodiment 123-124 to a subject in need thereof.
Embodiment 126. A chirally enriched population of oligomeric compounds of any of embodiments 1-120, wherein the population is enriched for oligomeric compounds comprising at least one particular phosphorothioate internucleoside linkage having a particular stereochemical configuration.
Embodiment 127. The chirally enriched population of embodiment 126, wherein the population is enriched for oligomeric compounds comprising at least one particular phosphorothioate internucleoside linkage having the (Sp) configuration.
Embodiment 128. The chirally enriched population of embodiment 126, wherein the population is enriched for oligomeric compounds comprising at least one particular phosphorothioate internucleoside linkage having the (Rp) configuration.
Embodiment 129. The chirally enriched population of embodiment 126, wherein the population is enriched for oligomeric compounds having a particular, independently selected stereochemical configuration at each phosphorothioate internucleoside linkage
Embodiment 130. The chirally enriched population of embodiment 126, wherein the population is enriched for oligomeric compounds having the (Sp) configuration at each phosphorothioate internucleoside linkage.
Embodiment 131. The chirally enriched population of embodiment 126, wherein the population is enriched for oligomeric compounds having the (Rp) configuration at each phosphorothioate internucleoside linkage.
Embodiment 132. The chirally enriched population of embodiment 126, wherein the population is enriched for oligomeric compounds having the (Rp) configuration at one particular phosphorothioate internucleoside linkage and the (Sp) configuration at each of the remaining phosphorothioate internucleoside linkages.
Embodiment 133. The chirally enriched population of embodiment 126 or embodiment 127 wherein the population is enriched for oligomeric compounds having at least 3 contiguous phosphorothioate internucleoside linkages in the Sp, Sp, and Rp configurations, in the 5′ to 3′ direction.
Embodiment 134. A chirally enriched population of modified oligonucleotides of any of embodiments 1-120, wherein all of the phosphorothioate internucleoside linkages of the modified oligonucleotide are stereorandom.
Embodiment 135. An oligomeric compound comprising an oligonucleotide and a conjugate group comprising a conjugate moiety, and a conjugate linker that links the conjugate moiety to the oligonucleotide, wherein the conjugate moiety comprises a cell-targeting peptide that binds human sortilin.
Embodiment 136. The oligomeric compound of embodiment 135, wherein the cell-targeting peptide comprises or consists of a human neurotensin peptide or a sortilin-binding portion thereof.
Embodiment 137. The oligomeric compound of embodiment 136, wherein the amino acid sequence of the human neurotensin peptide comprises or consists of an amino acid sequence selected from XLYENKPRRPYIL (SEQ ID NO: 3) LYENKPRRPYIL (SEQ ID NO: 32), ELYENKPRRPYIL (SEQ ID NO: 72), XLYENKPKRPYIL (SEQ ID NO: 73), XLYENKPRKPYIL (SEQ ID NO: 74), and XLYENKPKKPYIL (SEQ ID NO: 75), wherein X is lysine or 2-azido-acetyl lysine.
Embodiment 138. The oligomeric compound of embodiment 136, wherein the amino acid sequence of the human neurotensin peptide sequence is at least 75%, at least 80%, at least 85%, at least 90%, or at least 95% identical to an amino acid sequence selected from KLYENKPRRPYIL (SEQ ID NO: 3), LYENKPRRPYIL (SEQ ID NO: 32), ELYENKPRRPYIL (SEQ ID NO: 72), XLYENKPKRPYIL (SEQ ID NO: 73), XLYENKPRKPYIL (SEQ ID NO: 74), and XLYENKPKKPYIL (SEQ ID NO: 75), wherein X is lysine or 2-azido-acetyl lysine.
Embodiment 139. The oligomeric compound of embodiment 135, wherein the cell-targeting peptide comprises or consists of a sortilin propeptide or a sortilin-binding portion thereof.
Embodiment 140. The oligomeric compound of embodiment 139, wherein the amino acid sequence of the sortilin propeptide comprises or consists of an amino acid sequence selected from CQDRLDAPPPPAAPLPRWSGPIGVSWGLRAAAAGGAFPRGGRWRR (SEQ ID NO: 76) and CQDRLDAPPPPAAPLPRWSGPIGVSWGLR (SEQ ID NO: 77).
Embodiment 141. The oligomeric compound of embodiment 139, wherein the amino acid sequence of the sortilin propeptide is at least 75%, at least 80%, at least 85%, at least 90%, or at least 95% identical to an amino acid sequence selected from CQDRLDAPPPPAAPLPRWSGPIGVSWGLRAAAAGGAFPRGGRWRR (SEQ ID NO: 76) and CQDRLDAPPPPAAPLPRWSGPIGVSWGLR (SEQ ID NO: 77).
Embodiment 142. The oligomeric compound of any one of embodiments 139-141, wherein the conjugate moiety comprises a peptide extender that extends the cell-targeting peptide.
Embodiment 143. The oligomeric compound of embodiment 142, wherein the amino acid sequence of the peptide extender and cell-targeting peptide together is selected from: XPPPAGSSPGLYENKPRRPYIL (SEQ ID NO: 5), XPAPSGPSPGLYENKPRRPYIL (SEQ ID NO: 6), XAGSIKPPPAGSSPGLYENKPRRPYIL (SEQ ID NO: 7), and XAGMSGASAGLYENKPRRPYIL (SEQ ID NO: 8), wherein X is lysine of azido-acetyl lysine.
Embodiment 144. The oligomeric compound of embodiment 142, wherein the amino acid sequence of the peptide extender and cell-targeting peptide together is at least 75%, at least 80%, at least 85%, at least 90%, or at least 95% identical to an amino acid sequence selected from XPPPAGSSPGLYENKPRRPYIL (SEQ ID NO: 5), XPAPSGPSPGLYENKPRRPYIL (SEQ ID NO: 6), XAGSIKPPPAGSSPGLYENKPRRPYIL (SEQ ID NO: 7), and XAGMSGASAGLYENKPRRPYIL (SEQ ID NO: 8), wherein X is lysine of azido-acetyl lysine.
Embodiment 145. An oligomeric compound comprising an oligonucleotide and a conjugate group comprising a conjugate moiety and a conjugate linker,
wherein the conjugate moiety comprises a cell-targeting peptide and a peptide extender that extends the cell-targeting peptide,
wherein the conjugate linker links the conjugate moiety to the oligonucleotide via the peptide extender,
wherein the cell-targeting peptide is at least 70%, at least 75%, at least 80%, at least 85%, at least 90%, or at least 95% identical to a peptide encoded by a human gene.
Embodiment 146. The oligomeric compound of embodiment 145, wherein the peptide extender is less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, less than about 5% identical to a human peptide.
Embodiment 147. The oligomeric compound of embodiment 145, wherein the peptide extender does not comprise a human peptide.
Embodiment 148. The oligomeric compound of any one of embodiments 142-147, wherein the peptide extender comprises or consists essentially of 3 to 50, 3 to 45, 3 to 40, 3 to 35, 3 to 30, 3 to 25, 3 to 20, 3 to 15, 3 to 10, 6 to 50, 6 to 45, 6 to 40, 6 to 35, 6 to 30, 6 to 25, 6 to 20, 6 to 15, or 6 to 10 amino acids.
Embodiment 149. The oligomeric compound of any one of embodiments 142-148, wherein the peptide extender comprises at least 6, at least 7, at least 8, at least 9, or at least 10 amino acids.
Embodiment 150. The oligomeric compound of any one of embodiments 142-149, wherein the peptide extender comprises at least one amino acid selected from serine, proline, hydroxyproline, methionine, cysteine and tyrosine.
Embodiment 151. The oligomeric compound of embodiment 142, wherein the peptide extender comprises at least 2 or at least 3 amino acids selected from serine, proline, hydroxyproline, methionine, cysteine and tyrosine.
Embodiment 152. The oligomeric compound of embodiment 151, wherein the at least 2 or at least 3 amino acids are contiguous.
Embodiment 153. The oligomeric compound of any one of embodiments 142-152, wherein the peptide extender comprises three contiguous proline residues.
Embodiment 154. The oligomeric compound of any one of embodiments 142-153, wherein the peptide extender has a molecular weight of about 400 g/mol to about 1800 g/mol.
Embodiment 155. The oligomeric compound of any one of embodiments 142-147, wherein the length of the peptide extender is at least 15 Å in a solvent selected from water and phosphate buffered saline.
Embodiment 156. The oligomeric compound of any one of embodiments 142-148, wherein the conjugate linker is connected to the 5′ end of the oligonucleotide.
Embodiment 157. The oligomeric compound of any one of embodiments 142-148 wherein the conjugate linker is connected to the 3′ end of the oligonucleotide.
Embodiment 158. The oligomeric compound of embodiment 156 or 157, wherein the conjugate linker is connected to a carboxy terminus of the peptide extender.
Embodiment 159. The oligomeric compound of embodiment 156 or 157, wherein the conjugate linker is connected to an amino terminus of the peptide extender.
Embodiment 160. The oligomeric compound of any one of embodiments 142-159, wherein the peptide extender has an amino acid sequence selected from: KPPPAGSSPG (SEQ ID NO: 48), X₁PPPAGSSPG (SEQ ID NO: 49), X₁X₂PPAGSSPG (SEQ ID NO: 50), X₁P X₂PAGSSPG (SEQ ID NO: 51), X₁PP X₂AGSSPG (SEQ ID NO: 52), X₁PPP X₂GSSPG (SEQ ID NO: 53), X₁PPPA X₂SSPG (SEQ ID NO: 54), X₁PPPAG X₂SPG (SEQ ID NO: 55), X₁PPPAGS X₂PG (SEQ ID NO: 56), X₁PPPAGSS X₂G (SEQ ID NO: 57), and X₁PPPAGSSPX (SEQ ID NO: 58), wherein X₁ is selected from lysine, 2-azido-acetyl lysine, D-lysine, L-lysine, N⁶-(2-azido-acetyl)-D-lysine, and N⁶-(2-azido-acetyl)-L-lysine; and X₂ is any amino acid.
Embodiment 161. The oligomeric compound of any one of embodiments 142-159, wherein the peptide extender has an amino acid sequence that is at least 70%, at least 75%, at least 80%, at least 85%, at least 90%, at least 95%, or 100% identical to the amino acid sequence of KPPPAGSSPG (SEQ ID NO: 48).
Embodiment 162. The oligomeric compound of any one of embodiments 142-159, wherein the peptide extender has an amino acid sequence selected from: CPPPAGSSPG (SEQ ID NO: 59), XPPPAGSSPG (SEQ ID NO: 49), CXPPAGSSPG (SEQ ID NO: 60), CPXPAGSSPG (SEQ ID NO: 61), CPPXAGSSPG (SEQ ID NO: 62), CPPPXGSSPG (SEQ ID NO: 63), CPPPAXSSPG (SEQ ID NO: 64), CPPPAGXSPG (SEQ ID NO: 65), CPPPAGSXPG (SEQ ID NO: 66), CPPPAGSSXG (SEQ ID NO: 67), and CPPPAGSSPX (SEQ ID NO: 68), wherein X is any amino acid.
Embodiment 163. The oligomeric compound of any one of embodiments 142-159, wherein the peptide extender comprises an amino acid sequence that is at least 70%, at least 75%, at least 80%, at least 85%, at least 90%, at least 95%, or 100% identical to the amino acid sequence of CPPPAGSSPG (SEQ ID NO: 59).
Embodiment 164. The oligomeric compound of any one of embodiments 135-152, wherein the peptide extender is represented by amino acid sequence CAGSIKPPPAGSSPG (SEQ ID NO: 70) or KAGSIKPPPAGSSPG (SEQ ID NO: 71).
Embodiment 165. The oligomeric compound of any one of embodiments 142-159, wherein the peptide extender has an amino acid sequence selected from: XPAPSGPSPG (SEQ ID NO: 81), XAGSIKPPPAGSSPG (SEQ ID NO: 82), and XAGMSGASAG (SEQ ID NO: 83), wherein X is selected from lysine, cysteine, and N⁶-(2-azido-acetyl)-lysine.
Embodiment 166. The oligomeric compound of any one of embodiments 160, 161 and 165, wherein the lysine of the peptide extender is D-Lysine.
Embodiment 167. The oligomeric compound of any one of embodiments 142-166, wherein the peptide extender has a net charge of 0, 1, or 2 at neutral pH.
Embodiment 168. The oligomeric compound of any one of embodiments 142-159, wherein the peptide extender comprises a structure of the formula X_{L}—(X_{S})ₙ—X₁—X₂—X₃—X₄—X₅—X₆—X₇—X₈—X₉, wherein:
   X_{L} comprises a linker amino acid;
   n is from 0 to 5;
   at least one of X_{S} is independently selected from an aliphatic amino acid, lysine, and serine;
   at least one of X₁—X₉ is a neutral amino acid.
Embodiment 169. The oligomeric compound of embodiment 168, wherein each of X₁—X₉ is a neutral amino acid.
Embodiment 170. The oligomeric compound of embodiment 168 or 169, wherein the side chain of X_{L} comprises a reactive group.
Embodiment 171. The oligomeric compound of embodiment 170, wherein the side chain of X_{L} comprises a nucleophile.
Embodiment 172. The oligomeric compound of embodiment 170, wherein the side chain of X_{L} comprises an electrophile.
Embodiment 173. The oligomeric compound of embodiment 170, wherein the side chain of X_{L} comprises an amine or a sulfhydryl.
Embodiment 174. The oligomeric compound of embodiment 173, wherein X_{L} is lysine.
Embodiment 175. The oligomeric compound of embodiment 173, wherein X_{L} is cysteine.
Embodiment 176. The oligomeric compound of embodiment 168, wherein n is 0.
Embodiment 177. The oligomeric compound of any one of embodiments 138-175, wherein n is 4.
Embodiment 178. The oligomeric compound of embodiment 177, wherein (X_{S})₄ is Ala-Gly-Ser-Ile.
Embodiment 179. The oligomeric compound of embodiment 177, wherein each amino acid of (X_{S})₄ is independently selected from an aliphatic amino acid, lysine, and serine.
Embodiment 180. The oligomeric compound of embodiment 177, wherein at least one amino acid of (X_{S})₄ is not lysine.
Embodiment 181. The oligomeric compound of embodiment 177, wherein at least one amino acid of (X_{S})₄ is not arginine.
Embodiment 182. The oligomeric compound of embodiment 177, wherein (X_{S})₄ does not comprise a lysine.
Embodiment 183. The oligomeric compound of embodiment 177, wherein (X_{S})₄ does not comprise an arginine.
Embodiment 184. The oligomeric compound of any of embodiments 162-176, wherein each of X₁, X₂, and X₃ is selected from alanine, proline, glycine, cysteine, methionine, or tyrosine.
Embodiment 185. The oligomeric compound of embodiment 184, wherein X₁ is alanine or proline.
Embodiment 186. The oligomeric compound of embodiment 185, wherein X₁ is alanine.
Embodiment 187. The oligomeric compound of embodiment 185, wherein X₁ is proline.
Embodiment 188. The oligomeric compound of any of embodiments 184-187, wherein X₂ is proline, alanine, or glycine.
Embodiment 189. The oligomeric compound of embodiment 188, wherein X₂ is proline.
Embodiment 190. The oligomeric compound of embodiment 188, wherein X₂ is alanine.
Embodiment 191. The oligomeric compound of embodiment 188, wherein X₂ is glycine.
Embodiment 192. The oligomeric compound of any of embodiments 188-191, wherein X₃ is proline, serine, or methionine.
Embodiment 193. The oligomeric compound of embodiment 192, wherein X₃ is proline.
Embodiment 194. The oligomeric compound of embodiment 192, wherein X₃ is serine.
Embodiment 195. The oligomeric compound of embodiment 192, wherein X₃ is methionine.
Embodiment 196. The oligomeric compound of any of embodiments 168-195, wherein X₄ is an aliphatic amino acid, serine, or cysteine.
Embodiment 197. The oligomeric compound of any of embodiments 168-195, wherein X₄ is a compact amino acid.
Embodiment 198. The oligomeric compound of embodiment 196 or 197, wherein X₄ is selected from alanine, serine, glycine, or cysteine.
Embodiment 199. The oligomeric compound of any of embodiments 196-198, wherein X₄ is alanine.
Embodiment 200. The oligomeric compound of any of embodiments 196-198, wherein X₄ is serine.
Embodiment 201. The oligomeric compound of any of embodiments 168-200, wherein X₅ is glycine.
Embodiment 202. The oligomeric compound of any of embodiments 168-201, wherein X₆ is a compact amino acid.
Embodiment 203. The oligomeric compound of any of embodiments 168-202, wherein X₆ is selected from alanine, proline, serine, glycine, cysteine, methionine, or tyrosine.
Embodiment 204. The oligomeric compound of embodiment 202 or 203, wherein X₆ is serine.
Embodiment 205. The oligomeric compound of embodiment 202 or 203, wherein X₆ is proline.
Embodiment 206. The oligomeric compound of embodiment 202 or 203, wherein X₆ is alanine.
Embodiment 207. The oligomeric compound of any of embodiments 168-206, wherein X₇ is a compact amino acid.
Embodiment 208. The oligomeric compound of embodiment 207, wherein X₇ is serine.
Embodiment 209. The oligomeric compound of any of embodiments 168-208, wherein X₈ is a compact amino acid.
Embodiment 210. The oligomeric compound of any of embodiments 168-208, wherein X₈ is selected from alanine, proline, glycine, cysteine, methionine, or tyrosine.
Embodiment 211. The oligomeric compound of embodiment 209 or 210, wherein X₈ is proline.
Embodiment 212. The oligomeric compound of embodiment 209 or 210, wherein X₈ is alanine.
Embodiment 213. The oligomeric compound of any of embodiments 168-212, wherein X₉ is a compact amino acid.
Embodiment 214. The oligomeric compound of embodiment 213, wherein X₉ is glycine.
Embodiment 215. The oligomeric compound of any of embodiments 168-214, wherein the peptide extender comprises at least two cyclic amino acids.
Embodiment 216. The oligomeric compound of any of embodiments 168-215, wherein the peptide extender comprises at least three cyclic amino acids.
Embodiment 217. The oligomeric compound of embodiment 214 or 215, wherein the peptide extender comprises at least two prolines.
Embodiment 218. The oligomeric compound of embodiment 214 or 215, wherein the peptide extender comprises at least three prolines.
Embodiment 219. The oligomeric compound of embodiment 214 or 215, wherein the peptide extender comprises exactly three prolines.
Embodiment 220. The oligomeric compound of any of embodiments 168-219, wherein the peptide extender comprises at least five, at least six, at least seven, at least eight or at least nine compact amino acids.
Embodiment 221. The oligomeric compound of any of embodiments 168-219, wherein each of X₄, X₅, X₆, X₇, X₈, and X₉ are compact amino acids.
Embodiment 222. The oligomeric compound of any of embodiments 168-219, wherein at least five of X₄, X₅, X₆, X₇, X₈, and X₉ are compact amino acids.
Embodiment 223. The oligomeric compound of any of embodiments 168-219, wherein the peptide extender comprises at least two or at least three glycines.
Embodiment 224. The oligomeric compound of any of embodiments 168-219, wherein the peptide extender does not comprise more than two or more than three glycines.
Embodiment 225. The oligomeric compound of any of embodiments 168-224, wherein the peptide extender comprises at least two serines.
Embodiment 226. The oligomeric compound of any of embodiments 168-224, wherein the peptide extender comprises not more than two or more than three serines.
Embodiment 227. The oligomeric compound of any of embodiments 168-226, wherein the peptide extender does not comprise the sequence GSSG.
Embodiment 228. The oligomeric compound of any of embodiments 168-227, wherein the peptide extender does not comprise the sequence GSSS.
Embodiment 229. The oligomeric compound of any of embodiments 168-228, wherein the peptide extender does not comprise the sequence SSYG.
Embodiment 230. The oligomeric compound of any of embodiments 168-229, wherein the peptide extender comprises at least three different amino acid types selected from acidic, basic, aliphatic, cyclic, or aromatic.
Embodiment 231. The oligomeric compound of any of embodiments 168-230, wherein the peptide extender comprises at least four different amino acid types selected from acidic, basic, aliphatic, cyclic, or aromatic.
Embodiment 232. The oligomeric compound of any of embodiments 168-231, wherein the peptide extender comprises at least five different amino acid types selected from acidic, basic, aliphatic, cyclic, or aromatic.
Embodiment 233. The oligomeric compound of any of embodiments 168-232, wherein the peptide extender does not have a motif of ABAB, wherein A is an amino acid of one type and B is an amino acid of another type.
Embodiment 234. The oligomeric compound of any of embodiments 168-233, wherein the peptide extender does not have more than three consecutive amino acids of the same type.
Embodiment 235. The oligomeric compound of any of embodiments 168-234, wherein X₅ and X₉ are glycine.
Embodiment 236. The oligomeric compound of any of embodiments 168-235, wherein X₅ and X₉ are glycine and X₇ is serine.
Embodiment 237. The oligomeric compound of any of embodiments 168-234, wherein X₉ is linked to another amino acid by a peptide bond.
Embodiment 238. The oligomeric compound of any of embodiments 135-142 and 145-237, wherein the carboxy terminus of the peptide extender is connected to the amino terminus of the cell-targeting peptide.
Embodiment 239. The oligomeric compound of any of embodiments 135-142 and 145-23, wherein an amino terminus of the peptide extender is connected to a carboxy terminus of the peptide.
Embodiment 240. The oligomeric compound of any of embodiments 135-239, wherein the conjugate linker comprises maleimide.
Embodiment 241. The oligomeric compound of any of embodiments 135-239, wherein the conjugate linker and the modified oligonucleotide are connected via click chemistry.
Embodiment 242. The oligomeric compound of any one of embodiments 135-241, wherein the conjugate linker comprises at least one linker-nucleoside.
Embodiment 243. The oligomeric compound of embodiment 242, wherein the conjugate linker comprises three linker-nucleosides.
Embodiment 244. The oligomeric compound of embodiment 242 or 243, wherein the internucleoside linkage between the 3′-most linker-nucleoside and the oligonucleotide is a phosphodiester internucleoside linkage.
Embodiment 245. The oligomeric compound of any of embodiments 242-244, wherein each internucleoside linkage between linker-nucleosides in the conjugate linker is a phosphodiester internucleoside linkage.
Embodiment 246. The oligomeric compound of any of embodiments 135-245, wherein the oligonucleotide comprises at least one deoxyribonucleoside.
Embodiment 247. The oligomeric compound of any of embodiments 135-246, wherein the oligonucleotide comprises at least one ribonucleoside.
Embodiment 248. The oligomeric compound of any of embodiments 135-247, wherein the oligonucleotide consists of 8 linked nucleosides to 50 linked nucleosides.
Embodiment 249. The oligomeric compound of any of embodiments 135-248, wherein the oligonucleotide consists of 12 to 30 linked nucleosides.
Embodiment 250. The oligomeric compound of any of embodiments 135-249, wherein the oligonucleotide is a modified oligonucleotide comprising at least one of a modified internucleoside linkage, a modified sugar moiety, and a modified nucleobase.
Embodiment 251. The oligomeric compound of embodiment 250, wherein the modified sugar moiety is a non-bicyclic sugar moiety.
Embodiment 252. The oligomeric compound of embodiment 251, wherein the non-bicyclic sugar moiety comprises a 2′-MOE sugar moiety, a 2′-OMe sugar moiety, a 2′-NMA sugar moiety, or a 2′-F sugar moiety.
Embodiment 253. The oligomeric compound of embodiment 250, wherein the modified sugar moiety is a bicyclic sugar moiety.
Embodiment 254. The oligomeric compound of embodiment 253, wherein the bicyclic sugar moiety comprises a 2′-4′ bridge selected from —O—CH₂—; and —O—CH(CH₃)—.
Embodiment 255. The oligomeric compound of any of embodiments 250-254, wherein the oligonucleotide is a gapmer.
Embodiment 256. The oligomeric compound of any of embodiments 250-255, wherein the oligonucleotide has
   a 5′-region consisting of 1-6 linked nucleosides;
   a deoxy region consisting of 6-10 linked nucleosides; and
   a 3′-region consisting of 1-6 linked nucleosides; wherein
   each of the 5′-region nucleosides and each of the 3′-region nucleosides comprises a modified sugar moiety.
Embodiment 257. The oligomeric compound of embodiment 256, wherein the oligonucleotide has
   a 5′-region consisting of 3 linked nucleosides;
   a deoxy region consisting of 10 linked nucleosides; and
   a 3′-region consisting of 3 linked nucleosides; wherein
   each of the 5′-region nucleosides and each of the 3′-region nucleosides comprises a bicyclic sugar moiety, and each of the deoxy region nucleosides comprises a 2′-β-D-deoxyribosyl sugar moiety.
Embodiment 258. The oligomeric compound of embodiment 257, wherein each bicyclic nucleoside comprises a —O—CH(CH₃)-2′-4′ bridge.
Embodiment 259. The oligomeric compound of embodiment 250, wherein each nucleoside of the oligonucleotide comprises a modified sugar moiety.
Embodiment 260. The oligomeric compound of embodiment 259, wherein each modified sugar moiety is selected from 2′-OMe, 2′-MOE, and 2′-NMA.
Embodiment 261. The oligomeric compound of any of embodiments 250-260, wherein at least one modified internucleoside linkage is a phosphorothioate internucleoside linkage.
Embodiment 262. The oligomeric compound of embodiment 261, wherein each internucleoside linkage of the oligonucleotide is a phosphorothioate internucleoside linkage.
Embodiment 263. The oligomeric compound of embodiment 261, wherein each internucleoside linkage is independently selected from a phosphorothioate internucleoside linkage and a phosphodiester internucleoside linkage.
Embodiment 264. The oligomeric compound of embodiment 250, wherein the oligonucleotide is a phosphoramidite morpholino oligomer.
Embodiment 265. The oligomeric compound of any one of embodiments 135-264, wherein the oligonucleotide is a single stranded oligonucleotide.
Embodiment 266. An oligomeric duplex comprising an oligomeric compound of any of embodiments 135-265 and a second oligomeric compound comprising a second oligonucleotide.
Embodiment 267. The oligomeric duplex of embodiment 266, wherein the oligonucleotide of the oligomeric compound of any of embodiments 135-264 is complementary to a target nucleic acid.
Embodiment 268. The oligomeric duplex of embodiment 266, wherein the second oligonucleotide is complementary to a target nucleic acid.
Embodiment 269. The oligomeric duplex of embodiment 267 or 268, wherein the second oligomeric compound comprises a 5′-stabilized phosphate moiety.
Embodiment 270. The oligomeric duplex of embodiment 269, wherein the 5′-stabilized phosphate moiety is 5′-vinyl phosphonate.
Embodiment 271. The oligomeric duplex of any of embodiments 266-270, wherein the second oligonucleotide is a modified oligonucleotide comprising at least one of a modified internucleoside linkage, a modified sugar moiety, and a modified nucleobase.
Embodiment 272. The oligomeric duplex of embodiment 271, wherein the modified sugar moiety is a non-bicyclic sugar moiety.
Embodiment 273. The oligomeric duplex of embodiment 272, wherein the non-bicyclic sugar moiety comprises a 2′-MOE sugar moiety, a 2′-OMe sugar moiety, a 2′-NMA sugar moiety, or a 2′-F sugar moiety.
Embodiment 274. The oligomeric duplex of embodiment 271, wherein the modified sugar moiety is a bicyclic sugar moiety.
Embodiment 275. The oligomeric duplex of embodiment 274, wherein the bicyclic sugar moiety comprises a 2′-4′ bridge selected from —O—CH₂—; and —O—CH(CH₃)—.
Embodiment 276. The oligomeric duplex of any of embodiments 266-275, wherein the second oligonucleotide is a gapmer.
Embodiment 277. The oligomeric duplex of any of embodiments 266-276, wherein the second oligonucleotide has
   a 5′-region consisting of 1-6 linked nucleosides;
   a deoxy region consisting of 6-10 linked nucleosides; and
   a 3′-region consisting of 1-6 linked nucleosides; wherein
   each of the 5′-region nucleosides and each of the 3′-region nucleosides comprises a modified sugar moiety.
Embodiment 278. The oligomeric duplex of embodiment 277, wherein the second oligonucleotide has
   a 5′-region consisting of 3 linked nucleosides;
   a deoxy region consisting of 10 linked nucleosides; and
   a 3′-region consisting of 3 linked nucleosides; wherein
   each of the 5′-region nucleosides and each of the 3′-region nucleosides comprises a bicyclic sugar moiety, and each of the deoxy region nucleosides comprises a 2′-β-D-deoxyribosyl sugar moiety.
Embodiment 279. The oligomeric duplex of embodiment 278, wherein each bicyclic nucleoside comprises a —O—CH(CH₃)-2′-4′ bridge.
Embodiment 280. The oligomeric duplex of any of embodiments 266-279, wherein at least one internucleoside linkage of the second oligonucleotide is a modified internucleoside linkage.
Embodiment 281. The oligomeric duplex of embodiment 280, wherein at least one modified internucleoside linkage of the second oligonucleotide is a phosphorothioate internucleoside linkage.
Embodiment 282. The oligomeric duplex of embodiment 280, wherein at least one internucleoside linkage of the second oligonucleotide is a phosphodiester internucleoside linkage.
Embodiment 283. The oligomeric duplex of embodiment 280, wherein each internucleoside linkage of the second oligonucleotide is selected from a phosphodiester internucleoside linkage and a phosphorothioate internucleoside linkage.
Embodiment 284. The oligomeric compound of any of embodiments 135-265 or the oligomeric duplex of embodiments 266-283, wherein the peptide extender enhances uptake of the oligonucleotide into a cell relative to a control oligomeric compound or oligomeric duplex without the peptide extender, wherein the oligonucleotide and the conjugate moiety of the control oligomeric compound or oligomeric duplex are linked only by the conjugate linker.
Embodiment 285. An antisense compound comprising or consisting of an oligomeric compound of any of embodiments 135-265 or an oligomeric duplex of embodiments 266-283.
Embodiment 286. The antisense compound of embodiment 285, which is an RNAse H compound capable of reducing the amount of a nucleic acid target through the activation of RNAse H.
Embodiment 287. The antisense compound of embodiment 285, which is an RNAi compound capable of reducing the amount of a nucleic acid target through the activation of RISC/Ago2.
Embodiment 288. The antisense compound of embodiment 285, which is splice-modulating agent capable of modulating splicing of a nucleic acid target.
Embodiment 289. A pharmaceutical composition comprising the oligomeric compound of any of embodiments 135-265, the oligomeric duplex of any of embodiments 266-283, or the antisense compound of any of embodiments 285-288, and a pharmaceutically acceptable carrier or diluent.
Embodiment 290. The pharmaceutical composition of embodiment 289, wherein the pharmaceutically acceptable carrier or diluent comprises phosphate buffered saline.
Embodiment 291. A method comprising administering the oligomeric compound of any of embodiments 135-265, the oligomeric duplex of any of embodiments 266-283, or the antisense compound of any of embodiments 285-288, or the pharmaceutical composition of embodiment 155-156 to a subject in need thereof.
Embodiment 292. A chirally enriched population of oligomeric compounds of any of embodiments 135-265, wherein the population is enriched for oligomeric compounds comprising at least one particular phosphorothioate internucleoside linkage having a particular stereochemical configuration.
Embodiment 293. The chirally enriched population of embodiment 292, wherein the population is enriched for oligomeric compounds comprising at least one particular phosphorothioate internucleoside linkage having the (Sp) configuration.
Embodiment 294. The chirally enriched population of embodiment 292, wherein the population is enriched for oligomeric compounds comprising at least one particular phosphorothioate internucleoside linkage having the (Rp) configuration.
Embodiment 295. The chirally enriched population of embodiment 292, wherein the population is enriched for oligomeric compounds having a particular, independently selected stereochemical configuration at each phosphorothioate internucleoside linkage
Embodiment 296. The chirally enriched population of embodiment 292, wherein the population is enriched for oligomeric compounds having the (Sp) configuration at each phosphorothioate internucleoside linkage.
Embodiment 297. The chirally enriched population of embodiment 292, wherein the population is enriched for oligomeric compounds having the (Rp) configuration at each phosphorothioate internucleoside linkage.
Embodiment 298. The chirally enriched population of embodiment 292, wherein the population is enriched for oligomeric compounds having the (Rp) configuration at one particular phosphorothioate internucleoside linkage and the (Sp) configuration at each of the remaining phosphorothioate internucleoside linkages.
Embodiment 299. The chirally enriched population of embodiment 292, wherein the population is enriched for oligomeric compounds having at least 3 contiguous phosphorothioate internucleoside linkages in the Sp, Sp, and Rp configurations, in the 5′ to 3′ direction.
Embodiment 300. A chirally enriched population of modified oligonucleotides of any of embodiments 135-291, wherein all of the phosphorothioate internucleoside linkages of the modified oligonucleotide are stereorandom.

I. Certain Oligomeric Compounds

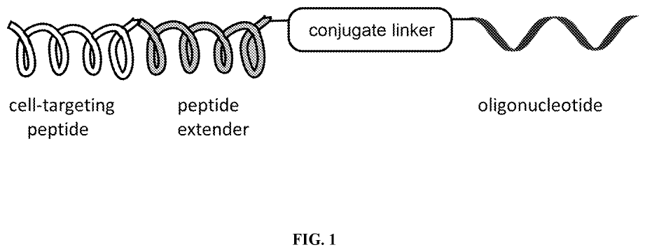

In certain embodiments, provided herein are oligomeric compounds comprising an oligonucleotide and one or more conjugate moieties. In certain embodiments, the oligonucleotide is a modified oligonucleotide. In certain embodiments, the oligonucleotide is an unmodified oligonucleotide. In certain embodiments, oligomeric compounds comprise an oligonucleotide, a cell-targeting moiety, a peptide extender, and a conjugate linker. In certain embodiments, the conjugate linker connects the peptide extender to the oligonucleotide, and the peptide extender connects the conjugate linker a cell-targeting peptide, as shown in FIG. 1. In certain embodiments, the oligonucleotide and the cell-targeting moiety are connected via the peptide extender and a conjugate linker, wherein the oligonucleotide is directly connected to the conjugate linker, the conjugate linker is directly connected to the peptide extender and the peptide extender is directly connected to the conjugate moiety.

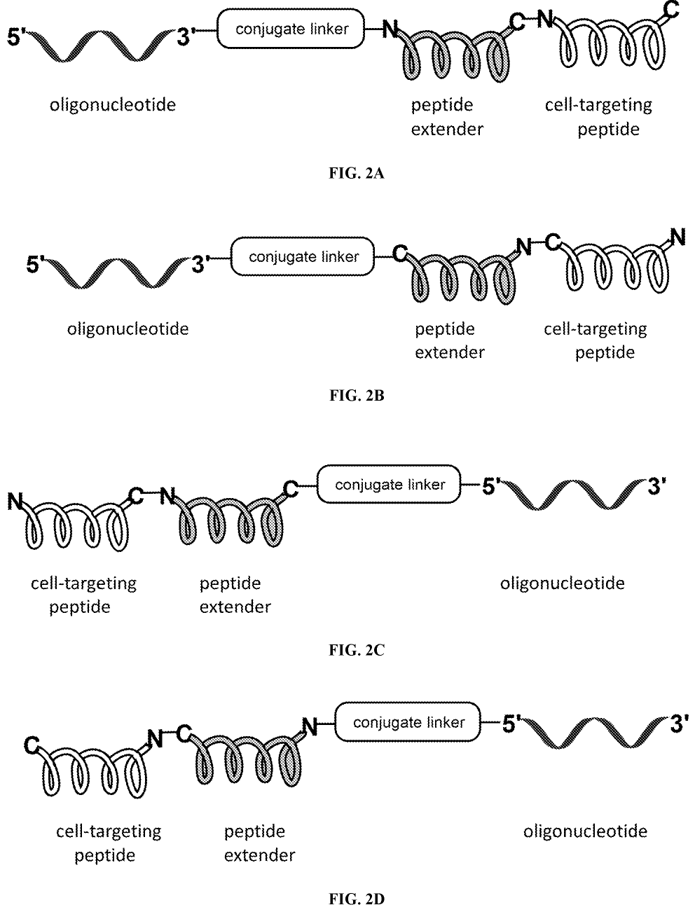

In certain embodiments, the cell-targeting peptide amino terminus is covalently connected to the peptide extender carboxy terminus, the peptide extender amino terminus is covalently linked to the conjugate linker, and the conjugate linker is covalently connected to the 3′ end of the oligonucleotide, see, e.g., FIG. 2A. In certain embodiments, the cell-targeting peptide carboxy terminus is covalently connected to the peptide extender amino terminus, the peptide extender carboxy terminus is covalently linked to the conjugate linker, and the conjugate linker is covalently connected to the 3′ end of the oligonucleotide, see, e.g., FIG. 2B. In certain embodiments, the cell-targeting peptide carboxy terminus is covalently connected to the peptide extender amino terminus, the peptide extender carboxy terminus is covalently linked to the conjugate linker, and the conjugate linker is covalently connected to the 5′ end of the oligonucleotide, see, e.g., FIG. 2C. In certain embodiments, the cell-targeting peptide amino terminus is covalently connected to the peptide extender carboxy terminus, the peptide extender amino terminus is covalently linked to the conjugate linker, and the conjugate linker is covalently connected to the 5′ end of the oligonucleotide, see, e.g., FIG. 2D.

In certain embodiments, the conjugate moiety is attached via the peptide extender and the conjugate linker to the 2′-position of a furanosyl sugar moiety of a nucleoside of an oligonucleotide. In certain such embodiments, the conjugate linker is attached to the 3′-end nucleoside of the oligonucleotide. In certain embodiments, the conjugate linker is attached to one of the two, three, four, five or six nucleosides closest to the 3′-end of the oligonucleotide. In certain embodiments, the conjugate linker is attached to the 5′-end nucleoside of the oligonucleotide. In certain embodiments, the conjugate linker is attached to one of the two, three, four, five or six nucleosides closest to the 5′-end of the oligonucleotide.

A. Certain Conjugate Moieties

In certain embodiments, conjugate moieties modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate moieties impart a new property on the attached oligonucleotide.

In certain embodiments, the conjugate moiety comprises a cell-targeting moiety. In certain embodiments, the cell-targeting moiety comprises a protein. In certain embodiments, the cell-targeting moiety consists of a protein. In certain embodiments, the protein consists of at least about 100, at least about 110, at least about 120, at least about 130, at least about 140, at least about 150, at least about 160, at least about 170, at least about 180, at least about 190 or at least about 200 amino acids. In certain embodiments, the conjugate moiety comprises a cell-targeting peptide. In certain embodiments, the conjugate moiety comprises a protein, wherein the protein comprises the cell-targeting peptide. In certain embodiments, the conjugate moiety consists of a cell-targeting peptide. In certain embodiments, the conjugate moiety comprises a cell-targeting peptide and a peptide extender. In certain embodiments, the conjugate moiety consists of a cell-targeting peptide and a peptide extender.

In certain embodiments, the cell-targeting peptide consists of 3 to 150, 4 to 150, 5 to 150, 6 to 150, 7 to 150, 8 to 150, 9 to 150, 10 to 150, 3 to 120, 4 to 120, 5 to 120, 6 to 120, 7 to 120, 8 to 120, 9 to 120, 10 to 120, 3 to 100, 4 to 100, 5 to 100, 6 to 100, 7 to 100, 8 to 100, 9 to 100, 10 to 100, 10 to 100 amino acids, 3 to 90, 4 to 90, 5 to 90, 6 to 90, 7 to 90, 8 to 90, 9 to 90, or 10 to 90 amino acids.

In certain embodiments, the cell-targeting peptide is capable of interacting with a cell surface receptor on a cell. In certain embodiments, the cell-targeting peptide is capable of interacting with a cell surface moiety on a cell. In certain embodiments, the cell-targeting peptide is capable of binding a cell surface receptor on a cell. In certain embodiments, the cell-targeting peptide is capable of binding a cell surface moiety on a cell. In certain embodiments, the cell-targeting peptide is capable of being internalized by the cell when it interacts with or binds the cell surface receptor or cell surface moiety. In certain embodiments, the cell surface receptor is not expressed ubiquitously (e.g., the cell surface receptor is undetectable in at least one tissue of a human subject), and the cell-targeting peptide selectively delivers an oligonucleotide to a tissue of interest or a cell of interest. By way of non-limiting example, the tissue of interest may be any one of brain, spinal cord, retina, heart, kidney, liver, lung, skeletal muscle, cardiac muscle, smooth muscle, adipose, white adipose, brown adipose, spleen, bone, intestine, colon, testes, breast, ovary, placenta, uterus, bladder, pancreas, pituitary, prostate, skin, adrenal gland, and thyroid. By way of non-limiting example, the cell of interest may be any one of a myocyte, adipocyte, hepatocyte, cardiomyocyte, vascular smooth muscle cell, endothelial cell, neuron, blood cell, macrophage, lymphocyte, cancer cell, and immune cell.

In certain embodiments, the cell-targeting peptide is capable of interacting with or binding any one of a ligand-gated ion channel, a G protein-coupled receptor, and a receptor tyrosine kinase. In certain embodiments, the cell surface receptor is capable of internalizing the cell-targeting peptide. In certain embodiments, the cell surface receptor is capable of internalizing an oligonucleotide connected to the cell-targeting peptide via a conjugate linker and peptide extender. In certain embodiments, the cell surface receptor is human sortilin, and the cell-targeting peptide is neurotensin, sortilin propeptide, or a sortilin-binding portion thereof.

Neurotensin In certain embodiments, the cell-targeting peptide is capable of interacting with sortilin. In certain embodiments, the cell-targeting peptide is capable of binding sortilin. In certain embodiments, the cell-targeting peptide comprises or consists of human neurotensin. In certain embodiments, the amino acid sequence of human neurotensin ELYENKPRRPYIL (SEQ ID NO: 72). In certain embodiments, the cell-targeting peptide comprises at least 5, at least 6, at least 7, at least 8, at least 9, at least 10, at least 11 or at least 12 contiguous amino acids that are identical to an equal length portion of SEQ ID NO: 72.

In certain embodiments, the cell-targeting peptide comprises human neurotensin. In certain embodiments, the cell-targeting peptide consists essentially of human neurotensin. In certain embodiments, the amino acid sequence of the cell-targeting peptide comprises an amino acid sequence selected from XLYENKPRRPYIL (SEQ ID NO: 3), LYENKPRRPYIL (SEQ ID NO: 32), ELYENKPRRPYIL (SEQ ID NO: 72), KLYENKPKRPYIL (SEQ ID NO: 73), KLYENKPRKPYIL (SEQ ID NO: 74), and KLYENKPKKPYIL (SEQ ID NO: 75), wherein X is lysine or 2-azido-acetyl lysine. In certain embodiments, the amino acid sequence of the cell-targeting peptide consists of an amino acid sequence selected from XLYENKPRRPYIL (SEQ ID NO: 3), LYENKPRRPYIL (SEQ ID NO: 32), ELYENKPRRPYIL (SEQ ID NO: 72), XLYENKPKRPYIL (SEQ ID NO: 73), XLYENKPRKPYIL (SEQ ID NO: 74), and XLYENKPKKPYIL (SEQ ID NO: 75) wherein X is lysine or 2-azido-acetyl lysine. In certain embodiments, the amino acid sequence of the cell-targeting peptide is at least 75%, at least 80%, at least 85%, at least 90%, or at least 95% identical to an amino acid sequence selected from XLYENKPRRPYIL (SEQ ID NO: 3), LYENKPRRPYIL (SEQ ID NO: 32), ELYENKPRRPYIL (SEQ ID NO: 72), XLYENKPKRPYIL (SEQ ID NO: 73), XLYENKPRKPYIL (SEQ ID NO: 74), and XLYENKPKKPYIL (SEQ ID NO: 75), wherein X is lysine or azido-acetyl lysine.

In certain embodiments, the conjugate moiety comprises or consists of a cell-targeting peptide and a peptide extender, wherein the amino acid sequence of the cell-targeting peptide and peptide extender together is selected from: XPPPAGSSPGLYENKPRRPYIL (SEQ ID NO: 5), XPAPSGPSPGLYENKPRRPYIL (SEQ ID NO: 6), XAGSIKPPPAGSSPGLYENKPRRPYIL (SEQ ID NO: 7), and XAGMSGASAGLYENKPRRPYIL (SEQ ID NO: 8), wherein X is lysine or 2-azido-acetyl lysine. In certain embodiments, the amino acid sequence of the cell-targeting peptide and peptide extender collectively is at least 75%, at least 80%, at least 85%, or at least 90% identical to an amino acid sequence selected from SEQ ID NOS: 6-8.

In certain embodiments, the cell-targeting peptide comprises a sortilin propeptide that is capable of binding to sortilin. In certain embodiments, the cell-targeting peptide consists essentially of a sortilin propeptide that is capable of binding to sortilin. Sortilin propeptides are described by Westergaard et al., (2004) Journal of Biological Chemistry 279:50221-50229. In certain embodiments, the amino acid sequence of the cell-targeting peptide comprises an amino acid sequence selected from QDRLDAPPPPAAPLPRWSGPIGVSWGLR (SEQ ID NO. 33) and QDRLDAPPPPAAPLPRWSGPIGVSWGLRAAAAGGAFPRGGRWRR (SEQ ID NO. 34). In certain embodiments, the amino acid sequence of the cell-targeting peptide consists of an amino acid sequence selected from QDRLDAPPPPAAPLPRWSGPIGVSWGLR (SEQ ID NO. 33) and QDRLDAPPPPAAPLPRWSGPIGVSWGLRAAAAGGAFPRGGRWRR (SEQ ID NO. 34). In certain embodiments, the amino acid sequence of the cell-targeting peptide is at least 75%, at least 80%, at least 85%, at least 90%, or at least 95% identical to an amino acid sequence selected from QDRLDAPPPPAAPLPRWSGPIGVSWGLR (SEQ ID NO. 33) and QDRLDAPPPPAAPLPRWSGPIGVSWGLRAAAAGGAFPRGGRWRR (SEQ ID NO. 34). In certain embodiments, the amino acid sequence of the cell-targeting peptide comprises an amino acid sequence selected from CQDRLDAPPPPAAPLPRWSGPIGVSWGLRAAAAGGAFPRGGRWRR (Propeptide 31, SEQ ID NO: 76) and CQDRLDAPPPPAAPLPRWSGPIGVSWGLR (Propeptide 30, SEQ ID NO: 77). In certain embodiments, the amino acid sequence of the cell-targeting peptide consists of an amino acid sequence selected from CQDRLDAPPPPAAPLPRWSGPIGVSWGLRAAAAGGAFPRGGRWRR (Propeptide 31, SEQ ID NO: 76) and CQDRLDAPPPPAAPLPRWSGPIGVSWGLR (Propeptide 30, SEQ ID NO: 77). In certain embodiments, the amino acid sequence of the cell-targeting peptide is at least 75%, at least 80%, at least 85%, at least 90%, or at least 95% identical to an amino acid sequence selected from CQDRLDAPPPPAAPLPRWSGPIGVSWGLRAAAAGGAFPRGGRWRR (Propeptide 31, SEQ ID NO: 76) and CQDRLDAPPPPAAPLPRWSGPIGVSWGLR (Propeptide 30, SEQ ID NO: 77).

In certain embodiments, the cell-targeting peptide comprises a cysteine at its amino terminus. In certain embodiments, the cell-targeting peptide comprises a cysteine at its carboxy terminus. In certain embodiments, the cysteine is directly linked to the modified oligonucleotide. In certain embodiments, the cysteine is indirectly linked to the modified oligonucleotide. For example, the oligomeric compound may comprise a conjugate linker, wherein the cysteine is directly linked to the conjugate linker at a first point on the conjugate linker and the modified oligonucleotide is directly linked to the conjugate linker at a second point on the conjugate linker, wherein the first point and the second point are different.

In certain embodiments, the cell-targeting peptide comprises a lysine at its amino terminus. In certain embodiments, the cell-targeting peptide comprises a lysine at its carboxy terminus. In certain embodiments, the lysine is directly linked to the modified oligonucleotide. In certain embodiments, the lysine is indirectly linked to the modified oligonucleotide. For example, the oligomeric compound may comprise a conjugate linker, wherein the lysine is directly linked to the conjugate linker at a first point on the conjugate linker and the modified oligonucleotide is directly linked to the conjugate linker at a second point on the conjugate linker, wherein the first point and the second point are different.

In certain embodiments, an amino terminal lysine of the cell-targeting peptide is modified to 2-azido-acetyl lysine to facilitate the conjugation of the conjugate moiety to the modified oligonucleotide. In certain embodiments, the amino acid sequence of the cell-targeting peptide is at least 80%, at least 85%, at least 90%, at least 95%, or 100% identical to an amino acid sequence selected from: SEQ ID NOS: 3, 6-8, and 73-75. The azido-acetyl group is attached to the side chain amine as shown below:

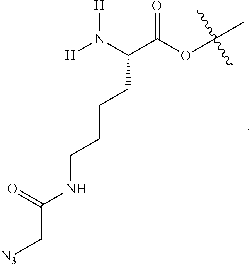 [SMILES: *OC(=O)[C@@H](N)CCCCNC(=O)CN=[N+]=[N-]]

Certain Peptide Extenders

In certain embodiments, oligomeric compounds comprise a peptide extender. In certain embodiments, the peptide extender is capable of providing a distance or barrier between the oligonucleotide and the cell-targeting moiety such that the oligonucleotide does not inhibit an activity of the cell-targeting moiety and the cell-targeting moiety does not inhibit an activity of the oligonucleotide. In certain embodiments, the peptide extender is capable of providing a distance or barrier between the oligonucleotide and the cell-targeting moiety such that the oligonucleotide inhibits an activity of the cell-targeting moiety to a lesser degree and/or the cell-targeting moiety inhibits an activity of the oligonucleotide to a lesser degree relative to the respective inhibition that would occur in the absence of the peptide extender. In certain embodiments, the activity of the cell-targeting moiety is binding a cell surface moiety (e.g., cell surface receptor). In certain embodiments, the activity of the oligonucleotide is an antisense activity.

In general, the peptide extender is not a cell-targeting moiety. In certain embodiments, the peptide extender does not interact with a cell-surface moiety. In certain embodiments, the cell-targeting moiety comprises a cell-targeting peptide, wherein the peptide extender and the cell-targeting peptide are not peptides encoded by the same species. In certain embodiments, the peptide extender is not a peptide encoded by a human gene.

In certain embodiments, the peptide extender comprises at least one amino acid selected from serine, proline, hydroxyproline, methionine, cysteine and tyrosine. In certain embodiments, the peptide extender comprises at least two, at least three or at least four amino acids selected from serine, proline, hydroxyproline, methionine, cysteine and tyrosine. In certain embodiments, the peptide extender comprises two contiguous amino acids selected from a serine, proline, hydroxyproline, methionine, cysteine and tyrosine. In certain embodiments, the peptide extender comprises three contiguous amino acids selected from a serine, proline, hydroxyproline, methionine, cysteine and tyrosine. In certain embodiments, the peptide extender comprises four contiguous amino acids selected from a serine, proline, hydroxyproline, methionine, cysteine and tyrosine. In certain embodiments, the peptide extender comprises a polyproline helix.

In certain embodiments, the peptide extender does not comprise more than 1 basic amino acid. In certain embodiments, the peptide extender does not comprise any basic amino acids. In certain embodiments, the peptide extender does not comprise more than one lysine or arginine. In certain embodiments, the peptide extender does not comprise a lysine or an arginine. In certain embodiments, the net charge of the peptide extender at pH=7 is less than or equal to 2.

In certain embodiments, the peptide extender has a molecular weight of about 400 g/mol to about 1800 g/mol, about 500 g/mol to about 1700 g/mol, about 600 g/mol to about 1600 g/mol, about 700 g/mol to about 1500 g/mol, or about 800 g/mol to about 1400 g/mol.

In certain embodiments, the peptide extender has a molecular weight of at least about 400 g/mol, at least about 425 g/mol, at least about 450 g/mol, at least about 475 g/mol, at least about 500 g/mol, at least about 525 g/mol, at least about 550 g/mol, at least about 575 g/mol, at least about 600 g/mol, at least about 625 g/mol, at least about 650 g/mol, at least about 675 g/mol, at least about 700 g/mol, at least about 725 g/mol, at least about 750 g/mol, at least about 775 g/mol, at least about 800 g/mol, at least about 825 g/mol, at least about 850 g/mol, at least about 875 g/mol, or at least about 900 g/mol.

In certain embodiments, the peptide extender has a molecular weight of about 400 g/mol, about 425 g/mol, about 450 g/mol, about 475 g/mol, about 500 g/mol, about 525 g/mol, about 550 g/mol, about 575 g/mol, about 600 g/mol, about 625 g/mol, about 650 g/mol, about 675 g/mol, about 700 g/mol, about 725 g/mol, about 750 g/mol, about 775 g/mol, about 800 g/mol, about 825 g/mol, about 850 g/mol, about 875 g/mol, about 900 g/mol, about 925 g/mol, about 950 g/mol, about 975 g/mol, about 1000 g/mol, about 1025 g/mol, about 1050 g/mol, about 1075 g/mol, about 1100 g/mol, about 1125 g/mol, about 1150 g/mol, about 1175 g/mol, about 1200 g/mol, about 1225 g/mol, about 1250 g/mol, about 1275 g/mol, about 1300 g/mol, about 1325 g/mol, about 1350 g/mol, about 1375 g/mol, about 1400 g/mol, about 1425 g/mol, about 1450 g/mol, about 1475 g/mol, about 1500 g/mol, about 1525 g/mol, about 1550 g/mol, about 1575 g/mol, about 1600 g/mol.

In certain embodiments, the peptide extender has a length selected from about 5 Å to about 10 Å, about 10 Å to about 15 Å, about 15 Å to about 20 Å, and about 20 Å to about 25 Å. In certain embodiments, the peptide extender has a length of at least 2 Å, at least 4 Å, at least 6 Å, at least 8 Å, at least 10 Å, at least 12 Å, at least 14 Å, at least 16 Å, at least 18 Å, at least 20 Å, at least 22 Å, or at least 24 Å. In certain embodiments, the peptide extender has a length selected from 10 Å, 11 Å, 12 Å, 13 Å, 14 Å, 15 Å, 16 Å, 17 Å, 18 Å, 19 Å, and 20 Å. In certain embodiments, the length of a peptide extender of an oligomeric compound is its length when the oligomeric compound is present in a solvent. In certain embodiments, the solvent is water. In certain embodiments, the solvent is a saline solution. In certain embodiments, the solvent is phosphate buffered saline (PBS).

In certain embodiments, the peptide extender comprises at least 1, at least 2, at least 3, at least 4, at least 5, at least 6, at least 7, at least 8, at least 9, at least 10, at least 11, or at least 12 amino acids. In certain embodiments, the peptide extender comprises 3 to 50, 3 to 45, 3 to 40, 3 to 35, 3 to 30, 3 to 25, 3 to 20, 3 to 15, 3 to 10, 6 to 50, 6 to 45, 6 to 40, 6 to 35, 6 to 30, 6 to 25, 6 to 20, 6 to 15, or 6 to 10 amino acids. In certain embodiments, the peptide extender comprises 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids. In certain embodiments, the peptide extender consists of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids. In certain embodiments, the peptide extender comprises 9 amino acids. In certain embodiments, the peptide extender consists of 9 amino acids. In certain embodiments, the peptide extender comprises 10 amino acids. In certain embodiments, the peptide extender consists of 10 amino acids.

In certain embodiments, the amino acid sequence of the peptide extender comprises the amino acid sequence of PPPAGSSPG (SEQ ID NO: 38). In certain embodiments, the amino acid sequence of the peptide extender is PPPAGSSPG (SEQ ID NO: 38). In certain embodiments, the amino acid sequence of the peptide extender comprises an amino acid sequence that is at least 75%, at least 80%, at least 85% identical to the amino acid sequence of PPPAGSSPG (SEQ ID NO: 38).

In certain embodiments, the amino acid sequence of the peptide extender comprises an amino acid sequence selected from: PPPAGSSPG (SEQ ID NO: 38), XPPAGSSPG (SEQ ID NO: 39), PXPAGSSPG (SEQ ID NO: 40), PPXAGSSPG (SEQ ID NO: 41), PPPXGSSPG (SEQ ID NO: 42), PPPAXSSPG (SEQ ID NO: 43), PPPAGXSPG (SEQ ID NO: 44), PPPAGSXPG (SEQ ID NO: 45), PPPAGSSXG (SEQ ID NO: 46), and PPPAGSSPX (SEQ ID NO: 47), wherein X is any amino acid. In certain embodiments, X is a nonpolar amino acid. In certain embodiments, X is a non-charged polar amino acid. In certain embodiments, X is a basic amino acid. In certain embodiments, X is an acidic amino acid. In certain embodiments, at least one serine is replaced with a threonine. In certain embodiments, alanine is replaced with valine, leucine, or isoleucine. In certain embodiments, X is not lysine. In certain embodiments, X is not arginine.

In certain embodiments, the peptide extender comprises a lysine at its amino terminus. In certain embodiments, the peptide extender comprises a lysine at its carboxy terminus. In certain embodiments, the lysine is directly linked to the modified oligonucleotide. In certain embodiments, the lysine is indirectly linked to the modified oligonucleotide. For example, the oligomeric compound may comprise a conjugate linker, wherein the lysine is directly linked to the conjugate linker at a first point on the conjugate linker and the modified oligonucleotide is directly linked to the conjugate linker at a second point on the conjugate linker, wherein the first point and the second point are different. In some embodiments, an amino terminal lysine of the peptide extender is modified to azido-acetyl lysine to facilitate the conjugation of peptide extender to the modified oligonucleotide. The azido-acetyl group is attached to the side chain amine as shown below:

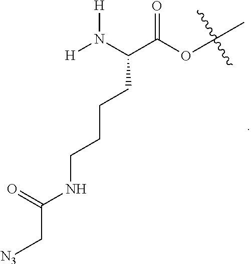 [SMILES: *OC(=O)[C@@H](N)CCCCNC(=O)CN=[N+]=[N-]]

In certain embodiments, the amino acid sequence of the peptide extender comprises or consists of an amino acid sequence selected from: KPPPAGSSPG (SEQ ID NO: 48), X₁PPPAGSSPG (SEQ ID NO: 49), X₁X₂PPAGSSPG (SEQ ID NO: 50), X₁P X₂PAGSSPG (SEQ ID NO: 51), X₁PP X₂AGSSPG (SEQ ID NO: 52), X₁PPP X₂GSSPG (SEQ ID NO: 53), X₁PPPA X₂SSPG (SEQ ID NO: 54), X₁PPPAG X₂SPG (SEQ ID NO: 55), X₁PPPAGS X₂PG (SEQ ID NO: 56), X₁PPPAGSS X₂G (SEQ ID NO: 57), and X₁PPPAGSSPX (SEQ ID NO: 58), wherein X₁ is selected from lysine, 2-azido-acetyl lysine, D-lysine, L-lysine, N⁶-(2-azido-acetyl)-D-lysine, and N⁶-(2-azido-acetyl)-L-lysine; and X₂ is any amino acid. In certain embodiments, X₂ is a nonpolar amino acid. In certain embodiments, X₂ is a non-charged polar amino acid. In certain embodiments, X₂ is a basic amino acid. In certain embodiments, X₂ is an acidic amino acid. In certain embodiments, at least one serine is replaced with a threonine. In certain embodiments, alanine is replaced with valine, leucine, or isoleucine. In certain embodiments, the amino acid sequence of the peptide extender is at least 75%, at least 80%, or at least 85% identical to an amino acid sequence selected from: KPPPAGSSPG (SEQ ID NO: 48), X₁PPPAGSSPG (SEQ ID NO: 49), X₁X₂PPAGSSPG (SEQ ID NO: 50), X₁P X₂PAGSSPG (SEQ ID NO: 51), X₁PP X₂AGSSPG (SEQ ID NO: 52), X₁PPP X₂GSSPG (SEQ ID NO: 53), X₁PPPA X₂SSPG (SEQ ID NO: 54), X₁PPPAG X₂SPG (SEQ ID NO: 55), X₁PPPAGS X₂PG (SEQ ID NO: 56), X₁PPPAGSS X₂G (SEQ ID NO: 57), and X₁PPPAGSSPX (SEQ ID NO: 58), wherein X₁ is selected from lysine, 2-azido-acetyl lysine, D-lysine, L-lysine, N⁶-(2-azido-acetyl)-D-lysine, and N⁶-(2-azido-acetyl)-L-lysine; and X₂ is any amino acid.

In certain embodiments, the amino acid of the peptide extender comprises an amino acid sequence of: CPPPAGSSPG (SEQ ID NO: 59). In certain embodiments, the amino acid of the peptide extender consists of an amino acid sequence of: CPPPAGSSPG (SEQ ID NO: 59). In certain embodiments, the peptide extender comprises an amino acid sequence that is at least 75% or at least 85% identical to the amino acid sequence of CPPPAGSSPG (SEQ ID NO: 59).

In certain embodiments, the amino acid sequence of the peptide extender comprises or consists of an amino acid sequence selected from: CPPPAGSSPG (SEQ ID NO: 59), XPPPAGSSPG (SEQ ID NO: 49), CXPPAGSSPG (SEQ ID NO: 60), CPXPAGSSPG (SEQ ID NO: 61), CPPXAGSSPG (SEQ ID NO: 62), CPPPXGSSPG (SEQ ID NO: 63), CPPPAXSSPG (SEQ ID NO: 64), CPPPAGXSPG (SEQ ID NO: 65), CPPPAGSXPG (SEQ ID NO: 66), CPPPAGSSXG (SEQ ID NO: 67), and CPPPAGSSPX (SEQ ID NO: 68) wherein X is any amino acid. In certain embodiments, X is a nonpolar amino acid. In certain embodiments, X is a non-charged polar amino acid. In certain embodiments, X is a basic amino acid. In certain embodiments, X is an acidic amino acid. In certain embodiments, at least one serine is replaced with a threonine. In certain embodiments, alanine is replaced with valine, leucine, or isoleucine. In certain embodiments, X is not lysine. In certain embodiments, X is not arginine.

In certain embodiments, the amino acid sequence of the peptide extender comprises or consists of an amino acid sequence selected from: XPAPSGPSPG (SEQ ID NO: 81), XAGSIKPPPAGSSPG (SEQ ID NO: 82), and XAGMSGASAG (SEQ ID NO: 83), wherein X is selected from lysine, cysteine, D-lysine, L-lysine, N⁶-(2-azido-acetyl)-lysine, N⁶-(2-azido-acetyl)-D-lysine, and N⁶-(2-azido-acetyl)-L-lysine. In certain embodiments, the amino acid sequence of the peptide extender is at least 75%, at least 80%, at least 85%, or at least 90% identical to an amino acid sequence selected from SEQ ID NOS: 6-8, wherein X is selected from lysine (K) and cysteine (C). In certain embodiments, the amino acid sequence of the peptide extender comprises an amino acid sequence having at least 3, at least 4, at least 5, at least 6, at least 7, or at least 8 consecutive amino acids that are identical to an equal length portion of the amino acid sequence of any one of SEQ ID NOS: 38-68. In certain embodiments, the amino acid sequence of the peptide extender comprises or consists of an amino acid sequence selected from CAGSIKPPPAGSSPG (SEQ ID NO: 70) or KAGSIKPPPAGSSPG (SEQ ID NO: 71).

In certain embodiments, the peptide extender comprises a linker amino acid that links the peptide extender to the conjugate linker. In certain embodiments, the linker amino acid is selected from lysine, cysteine, azido norleucine, and methionine. For example, the conjugate linker may be formed by click chemistry and the linker amino acid is lysine. Also, by way of example, the conjugate linker may comprise maleimide and the linker amino acid is cysteine. In certain embodiments, the peptide extender comprises the linker amino acid at its amino terminus. In certain embodiments, the peptide extender comprises the linker amino acid at its carboxy terminus. By way of non-limiting example, the peptide extender may be represented by the sequence: KPPPAGSSPG (SEQ ID NO: 48), wherein “K” (Lysine) is the linker amino acid. In certain embodiments, linker amino acid is selected from D-lysine, L-lysine, N⁶-(2-azido-acetyl)-D-lysine, and N⁶-(2-azido-acetyl)-L-lysine. In certain embodiments, the linker amino acid is D-Lysine, which improves the stability of the peptide as compared to when the linker amino acid is L-Lysine.

B. Certain Conjugate Linkers

In certain embodiments, oligomeric compounds comprise an oligonucleotide and a conjugate group, wherein the conjugate group comprises a conjugate moiety and a conjugate linker. In certain embodiments, the conjugate moiety comprises a peptide extender, wherein the conjugate linker links the peptide extender to the oligonucleotide. In certain embodiments, the conjugate linker is a single chemical bond (i.e., the peptide linker is attached directly to an oligonucleotide through a single bond). In certain embodiments, the conjugate linker comprises one or more atoms. In certain embodiments, the conjugate linker comprises a chemical group. In certain embodiments, the conjugate linker comprises a chain structure, such as a hydrocarbyl chain, or an oligomer of repeating units such as ethylene glycol, nucleosides, or amino acid units. In certain embodiments, the oligonucleotide is a modified oligonucleotide.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate moieties to parent compounds, such as the oligonucleotides provided herein. In general, a bifunctional linking moiety comprises at least two functional groups. One of the functional groups is selected to react with particular site on a parent compound and the other is selected to react with a peptide extender. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methyl cytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a oligonucleotide consisting of 8-30 nucleosides and (2) a conjugate linker comprising 1-10 linker-nucleosides that are contiguous with the nucleosides of the oligonucleotide. The total number of contiguous linked nucleosides in such an oligomeric compound is more than 30. Alternatively, an oligomeric compound may comprise a oligonucleotide consisting of 8-30 nucleosides and no conjugate linker. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate moiety to be cleaved from the oligonucleotide. For example, in certain circumstances oligomeric compounds comprising a particular conjugate moiety are better taken up by a particular cell type, but once the oligomeric compound has been taken up, it is desirable that the conjugate moiety be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphodiester linkage between an oligonucleotide and a conjugate moiety.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

In certain embodiments, oligomeric compounds disclosed herein comprise an oligonucleotide linked to conjugate moiety by a conjugate linker, wherein the oligomeric compound is prepared using Click chemistry known in the art. Compounds have been prepared using Click chemistry wherein alkynyl phosphonate internucleoside linkages on an oligomeric compound attached to a solid support are converted into the 1,2,3-triazolylphosphonate internucleoside linkages and then cleaved from the solid support (Krishna et al., J. Am. Chem. Soc. 2012, 134(28), 11618-11631), which is incorporated by reference herein in its entirety. Additional conjugate linkers suitable for use in several embodiments can be prepared by Click chemistry described in “Click Chemistry for Biotechnology and Materials Science” Ed. Joerg Laham, Wiley 2009, which is incorporated by reference herein in its entirety.

In certain embodiments, a Click reaction can be used to link a conjugate moiety and an oligonucleotide by reacting:

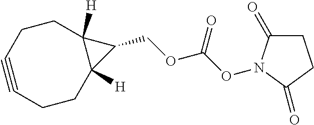 [SMILES: O=C(OC[C@@H]1[C@@H]2CCC#CCC[C@@H]21)ON1C(=O)CCC1=O]

with an oligonucleotide having a terminal amine, including but not limited to the following compound:

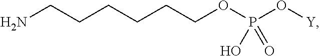

wherein Y is the remainder of the oligonucleotide, to yield:

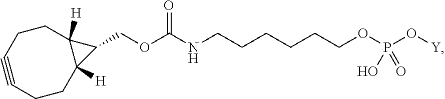

which can be reacted with a conjugate moiety having an azide to yield:

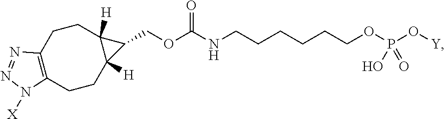

wherein N—N═N is formed from an azido group of the conjugate moiety, and wherein X represents the remainder of the conjugate moiety. In certain embodiments, the conjugate moiety comprises a peptide extender and/or a cell-targeting peptide. In certain embodiments, the azido group is attached to an amino-acid side chain of the peptide extender. In certain embodiments, the azido group replaces the amino group of a lysine of the peptide extender.

In certain embodiments, an oligomeric compound comprises an oligonucleotide linked to a conjugate moiety by a conjugate linker, wherein the conjugate linker is prepared from the following compound:

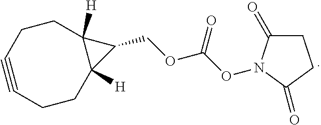 [SMILES: O=C(OC[C@@H]1[C@@H]2CCC#CCC[C@@H]21)ON1C(=O)CCC1=O]

In certain embodiments, an oligomeric compound comprises an oligonucleotide linked to a conjugate moiety by a conjugate linker, wherein the conjugate linker comprises:

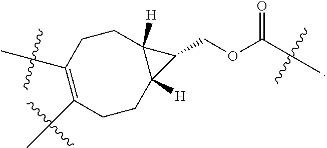 [SMILES: *C(=O)OC[C@@H]1[C@@H]2CC/C(*)=C(/*)CC[C@@H]21]

In certain embodiments, an oligomeric compound comprises an oligonucleotide linked to a conjugate moiety by a conjugate linker, wherein the conjugate linker comprises:

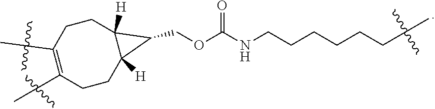 [SMILES: *CCCCCCNC(=O)OC[C@@H]1[C@@H]2CC/C(*)=C(/*)CC[C@@H]21]

In certain embodiments, an oligomeric compound comprises an oligonucleotide linked to a conjugate moiety by a conjugate linker, wherein the compound comprises:

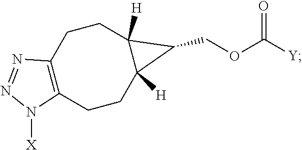

wherein N—N═N is formed from an azido group of the conjugate moiety; X represents the remainder of the conjugate moiety; and Y represents a portion of the oligomeric compound comprising the oligonucleotide. In certain embodiments, the conjugate moiety comprises a peptide extender and/or a cell-targeting peptide. In certain embodiments, the azido group is attached to an amino-acid side chain of the peptide extender. In certain embodiments, the azido group replaces the amino group of a lysine of the peptide extender.

In certain embodiments, an oligomeric compound comprises an oligonucleotide linked to a conjugate moiety by a conjugate linker, wherein the oligomeric compound comprises:

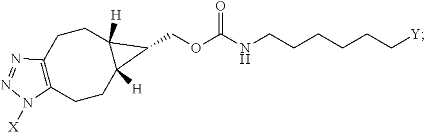

wherein N—N═N is formed from an azido group of the conjugate moiety; X represents the remainder of the conjugate moiety; and Y represents the remainder of the oligonucleotide. In certain embodiments, the conjugate moiety comprises a peptide extender and/or a cell-targeting peptide. In certain embodiments, the azido group is attached to an amino-acid side chain of the peptide extender. In certain embodiments, the azido group replaces the amino group of a lysine of the peptide extender.

In certain embodiments, an oligomeric compound comprises an oligonucleotide linked to a conjugate moiety by a conjugate linker, wherein the oligomeric compound comprises:

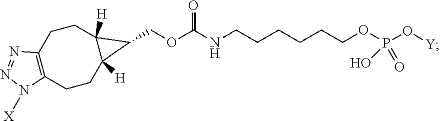

wherein N—N═N is formed from an azido group of the conjugate moiety; X represents the remainder of the conjugate moiety; and Y represents the remainder of the oligonucleotide. In certain embodiments, the conjugate moiety comprises a peptide extender and/or a cell-targeting peptide. In certain embodiments, the azido group is attached to an amino-acid side chain of the peptide extender. In certain embodiments, the azido group replaces the amino group of a lysine of the peptide extender.

In certain embodiments, an oligomeric compound comprises an oligonucleotide linked to a conjugate moiety by a conjugate linker, wherein the conjugate linker is prepared using Click chemistry and disulfide linkages.

In certain embodiments, an oligomeric compound comprises an oligonucleotide linked to a conjugate moiety by a conjugate linker, wherein the oligomeric compound comprises:

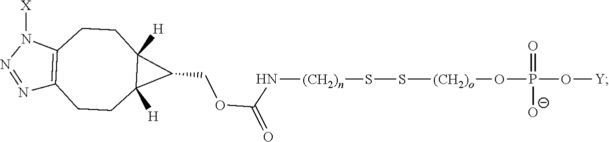

wherein N—N═N is formed from an azido group of the conjugate moiety; X represents the remainder of the conjugate moiety; n and o are independently selected from 2 to 10; and Y represents the remainder of the oligonucleotide. In certain embodiments, the conjugate moiety comprises a peptide extender and/or a cell-targeting peptide. In certain embodiments, the azido group is attached to an amino-acid side chain of the peptide extender. In certain embodiments, the azido group replaces the amino group of a lysine of the peptide extender.

In certain embodiments, an oligomeric compound comprises an oligonucleotide linked to a conjugate moiety by a conjugate linker, wherein the oligomeric compound comprises:

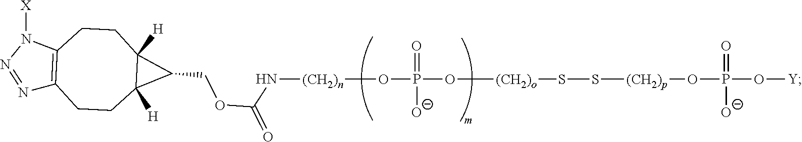

wherein N—N═N is formed from an azido group of the conjugate moiety; X represents the remainder of the conjugate moiety; n, o, and p are independently selected from 2 to 10; m is 0 or 1; and Y represents the remainder of the oligonucleotide. In certain embodiments, the conjugate moiety comprises a peptide extender and/or a cell-targeting peptide. In certain embodiments, the azido group is attached to an amino-acid side chain of the peptide extender. In certain embodiments, the azido group replaces the amino group of a lysine of the peptide extender.

In certain embodiments, an oligomeric compound comprises an oligonucleotide linked to a conjugate moiety by a conjugate linker, wherein the oligomeric compound comprises:

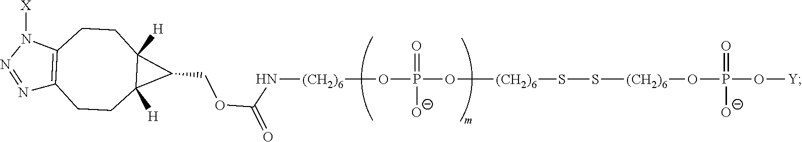

wherein N—N═N is formed from an azido group of the conjugate moiety; X represents the remainder of the conjugate moiety; m is 0 or 1; and Y represents the remainder of the oligonucleotide. In certain embodiments, the conjugate moiety comprises a peptide extender and/or a cell-targeting peptide. In certain embodiments, the azido group is attached to an amino-acid side chain of the peptide extender. In certain embodiments, the azido group replaces the amino group of a lysine of the peptide extender.

In certain embodiments, an oligomeric compound comprises an oligonucleotide linked to a conjugate moiety by a conjugate linker, wherein the oligomeric compound comprises:

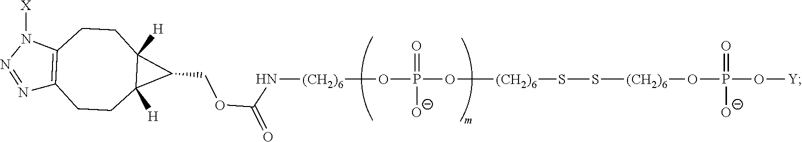

wherein N—N═N is formed from an azido group of the conjugate moiety; X represents the remainder of the conjugate moiety; m is 1; and Y represents the remainder of the oligonucleotide. In certain embodiments, the conjugate moiety comprises a peptide extender and/or a cell-targeting peptide. In certain embodiments, the azido group is attached to an amino-acid side chain of the peptide extender. In certain embodiments, the azido group replaces the amino group of a lysine of the peptide extender.

In certain embodiments, an oligomeric compound comprises an oligonucleotide linked to a conjugate moiety by a conjugate linker, wherein the compound comprises:

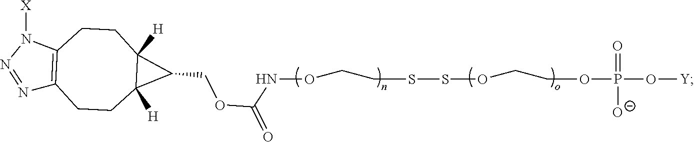

wherein N—N═N is formed from an azido group of the conjugate moiety; X represents the remainder of the conjugate moiety; n and o are independently selected from 2 to 10; and Y represents the remainder of the oligonucleotide. In certain embodiments, the conjugate moiety comprises a peptide extender and/or a cell-targeting peptide. In certain embodiments, the azido group is attached to an amino-acid side chain of the peptide extender. In certain embodiments, the azido group replaces the amino group of a lysine of the peptide extender.

In certain embodiments, an oligomeric compound comprises an oligonucleotide linked to a conjugate moiety by a conjugate linker, wherein the oligomeric compound comprises:

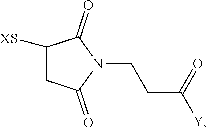

wherein X comprises the conjugate moiety; and Y comprises the oligonucleotide. In certain embodiments, the conjugate moiety comprises a cell-targeting peptide and a peptide extender, wherein the peptide extender links the cell-targeting peptide to the conjugate linker.

In certain embodiments, an oligomeric compound comprises an oligonucleotide linked to a conjugate moiety by a conjugate linker, wherein the compound comprises:

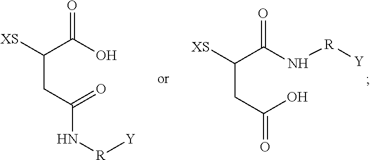

wherein R═(CH₂)ₙ and n is from 1 to 12; X comprises the conjugate moiety; and Y comprises the oligonucleotide. In certain embodiments, the conjugate moiety comprises a cell-targeting peptide and a peptide extender, wherein the peptide extender links the cell-targeting peptide to the conjugate linker.

In certain embodiments, an oligomeric compound comprises an oligonucleotide linked to a peptide extender by a conjugate linker, wherein the compound comprises:

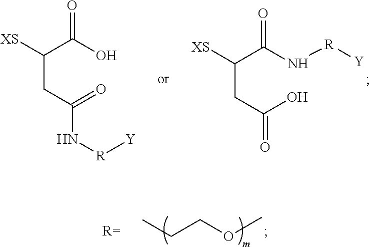

wherein m is from 1 to 12; X comprises the conjugate moiety; and Y comprises the oligonucleotide. In certain embodiments, the conjugate moiety comprises a cell-targeting peptide and a peptide extender, wherein the peptide extender links the cell-targeting peptide to the conjugate linker.

In certain embodiments, a composition comprises or consists of a substantially pure mixture of two oligomeric compounds, wherein the first oligomeric compound comprises a first oligonucleotide linked to a first conjugate moiety by a first conjugate linker, wherein the first oligomeric compound comprises:

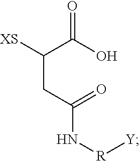

wherein R═(CH₂)ₙ and n is from 1 to 12; X comprises the first conjugate moiety; and Y comprises the first oligonucleotide; and
the second oligomeric compound comprises a second oligonucleotide linked to a second conjugate moiety by a second conjugate linker, wherein the second oligomeric compound comprises:

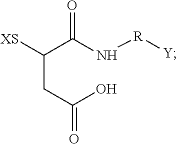

wherein R═(CH₂)ₙ and n is from 1 to 12; X comprises the second conjugate moiety; and Y comprises the second oligonucleotide. In certain embodiments, at least one of the first conjugate moiety and the second conjugate moiety comprises a cell-targeting peptide and a peptide extender, wherein the peptide extender links the cell-targeting peptide to the first conjugate linker or the second conjugate linker, respectively.

In certain embodiments, a composition comprises or consists of a substantially pure mixture of two oligomeric compounds, wherein the first oligomeric compound comprises a first oligonucleotide linked to a first conjugate moiety by a first conjugate linker, wherein the first oligomeric compound comprises:

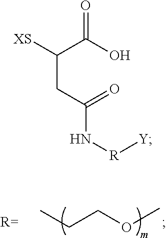

wherein m is from 1 to 12; X comprises the first conjugate moiety; and Y comprises the first oligonucleotide; and
the second oligomeric compound comprises a second oligonucleotide linked to a second conjugate moiety by a second conjugate linker, wherein the second oligomeric compound comprises:

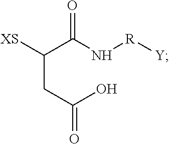

wherein

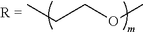

and m is from 1 to 12; X comprises the second conjugate moiety; and Y comprises the second oligonucleotide. In certain embodiments, at least one of the first conjugate moiety and the second conjugate moiety comprises a cell-targeting peptide and a peptide extender, wherein the peptide extender links the cell-targeting peptide to the first conjugate linker or the second conjugate linker, respectively.

In certain embodiments, an oligomeric compound comprises an oligonucleotide linked to a conjugate moiety by a conjugate linker, wherein the oligomeric compound comprises:

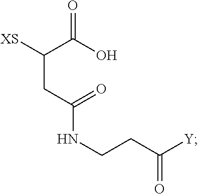

wherein X comprises the conjugate moiety; and Y comprises the oligonucleotide. In certain embodiments, the conjugate moiety comprises a cell-targeting peptide and a peptide extender, wherein the peptide extender links the cell-targeting peptide to the conjugate linker.

In certain embodiments, an oligomeric compound comprises an oligonucleotide linked to a conjugate moiety by a conjugate linker, wherein the oligomeric compound comprises:

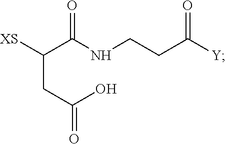

wherein X comprises the conjugate moiety; and Y comprises the oligonucleotide. In certain embodiments, the conjugate moiety comprises a cell-targeting peptide and a peptide extender, wherein the peptide extender links the cell-targeting peptide to the conjugate linker.

In certain embodiments, a composition comprises or consists of a substantially pure mixture of two oligomeric compounds, wherein the first oligomeric compound comprises a first oligonucleotide linked to a first conjugate moiety by a first conjugate linker, wherein the first oligomeric compound comprises:

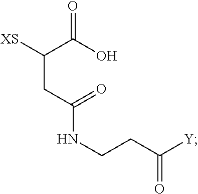

wherein X comprises the first conjugate moiety; and Y comprises the first oligonucleotide; and
the second oligomeric compound comprises a second oligonucleotide linked to a second conjugate moiety by a second conjugate linker, wherein the second oligomeric compound comprises:

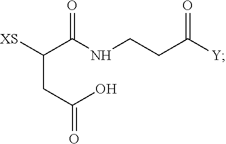

wherein X comprises the second conjugate moiety; and Y comprises the second oligonucleotide. In certain embodiments, at least one of the first conjugate moiety and the second conjugate moiety comprises a cell-targeting peptide and a peptide extender, wherein the peptide extender links the cell-targeting peptide to the first conjugate linker or the second conjugate linker, respectively.

In certain embodiments, an oligomeric compound comprises an oligonucleotide linked to a conjugate moiety by a conjugate linker, wherein the conjugate linker comprises a disulfide linkage. In certain embodiments, the conjugate moiety comprises a peptide extender, wherein the peptide extender is linked to the oligonucleotide via the disulfide bridge. In certain embodiments, the disulfide bridge directly connects the oligonucleotide to the peptide extender.

In certain embodiments, the conjugate linker does not comprise an amino acid.

II. Certain Oligonucleotides

In certain embodiments, provided herein are oligomeric compounds comprising oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage.

A. Certain Modified Nucleosides

Modified nucleosides comprise a modified sugar moiety or a modified nucleobase or both a modifed sugar moiety and a modified nucleobase.

1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more substituent groups none of which bridges two atoms of the furanosyl ring to form a bicyclic structure. Such non bridging substituents may be at any position of the furanosyl, including but not limited to substituents at the 2′, 4′, and/or 5′ positions. In certain embodiments one or more non-bridging substituent of non-bicyclic modified sugar moieties is branched. Examples of 2′-substituent groups suitable for non-bicyclic modified sugar moieties include but are not limited to: 2′-F, 2′-OCH₃(“OMe” or “O-methyl”), and 2′-O(CH₂)₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugar moieties comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.).

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCH₃, and OCH₂CH₂OCH₃.

Certain modifed sugar moieties comprise a substituent that bridges two atoms of the furanosyl ring to form a second ring, resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt”), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′C(—RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, C(═—NRₐ)—, —C(═O)—, —C(═S)—, —O—, —Si(Rₐ)₂—, —S(═O)ₓ—, and —N(Rₐ)—;
wherein: x is 0, 1, or 2; n is 1, 2, 3, or 4; each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J Org. Chem., 1998, 63, 10035-10039; Srivastava et al., J Am. Chem. Soc., 20017, 129, 8362-8379; Wengel et al., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

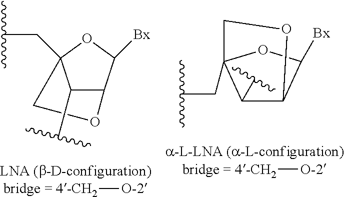

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, C J. Bioorg. & Med. Chem. 2002, 10, 841-854), fluoro HNA:

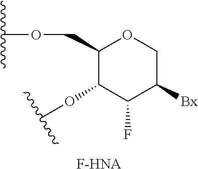

(“F-HNA”, see e.g., Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

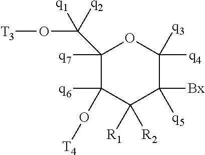

wherein, independently, for each of said modified THP nucleoside:
Bx is a nucleobase moiety;
T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate moiety, or a 5′ or 3-terminal group; q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

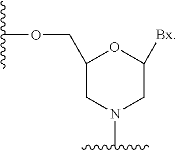

In certain embodiments, morpholinos may be modified, for example by adding or altering various substituent groups from the above morpholino structure. Such sugar surrogates are referred to herein as “modifed morpholinos.”

In certain embodiments, sugar surrogates comprise acyclic moieites. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides (see for example review article: Leumann, Bioorg. Med. Chem., 2002, 10, 841-854).

2. Certain Modified Nucleobases

In certain embodiments, modified oligonucleotides comprise one or more nucleosides comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

3. Certain Modified Internucleoside Linkages

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphodiesters (“P═O”) (also referred to as unmodified or naturally occurring linkages or phosphate linkages), phosphotriesters, methylphosphonates, phosphoramidates, and phosphorothioates (“P═S”), and phosphorodithioates (“HS-P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphodiester linkages, can be used to alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

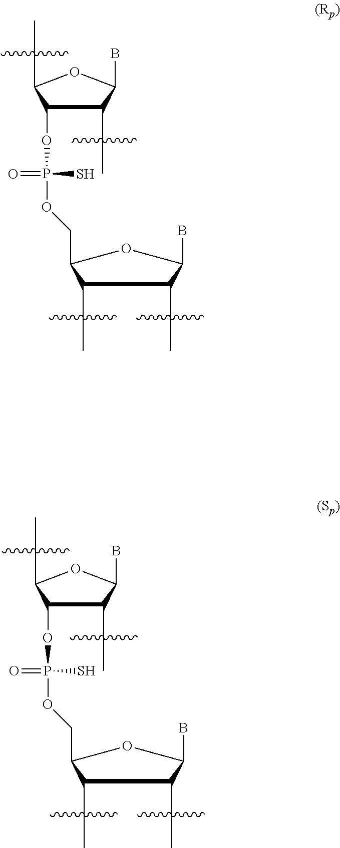

Unless otherwise indicated, chiral internucleoside linkages of modified oligonucleotides described herein can be stereorandom or in a particular stereochemical configuration.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

B. Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

1. Certain Sugar Motifs

In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides comprise or consist of a region having a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-5 nucleosides. In certain embodiments, each nucleoside of each wing of a gapmer is a modified nucleoside. In certain embodiments, at least one nucleoside of each wing of a gapmer is a modified nucleoside. In certain embodiments, at least two nucleosides of each wing of a gapmer are modified nucleosides. In certain embodiments, at least three nucleosides of each wing of a gapmer are modified nucleosides. In certain embodiments, at least four nucleosides of each wing of a gapmer are modified nucleosides.

In certain embodiments, the gap of a gapmer comprises 7-12 nucleosides. In certain embodiments, each nucleoside of the gap of a gapmer is an unmodified 2′-deoxy nucleoside.

In certain embodiments, the gapmer is a deoxy gapmer. In embodiments, the nucleosides on the gap side of each wing/gap junction are unmodified 2′-deoxy nucleosides and the nucleosides on the wing sides of each wing/gap junction are modified nucleosides. In certain embodiments, each nucleoside of the gap is an unmodified 2′-deoxy nucleoside. In certain embodiments, each nucleoside of each wing of a gapmer is a modified nucleoside.

In certain embodiments, modified oligonucleotides comprise or consist of a region having a fully modified sugar motif. In such embodiments, each nucleoside of the fully modified region of the modified oligonucleotide comprises a modified sugar moiety. In certain embodiments, each nucleoside of the entire modified oligonucleotide comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified comprises the same 2′-modification.

Herein, the lengths (number of nucleosides) of the three regions of a gapmer may be provided using the notation [#of nucleosides in the 5′-wing]−[#of nucleosides in the gap]−[#of nucleosides in the 3′-wing]. Thus, a 5-10-5 gapmer consists of 5 linked nucleosides in each wing and 10 linked nucleosides in the gap. Where such nomenclature is followed by a specific modification, that modification is the modification in each sugar moiety of each wing and the gap nucleosides comprise unmodified deoxynucleoside sugars. Thus, a 5-10-5 MOE gapmer consists of 5 linked MOE modified nucleosides in the 5′-wing, 10 linked deoxynucleosides in the gap, and 5 linked MOE nucleosides in the 3′-wing.

In certain embodiments, modified oligonucleotides are 5-10-5 MOE gapmers. In certain embodiments, modified oligonucleotides are 3-10-3 BNA gapmers. In certain embodiments, modified oligonucleotides are 3-10-3 cEt gapmers. In certain embodiments, modified oligonucleotides are 3-10-3 LNA gapmers.

2. Certain Nucleobase Motifs

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methyl cytosines. In certain embodiments, all of the cytosine nucleobases are 5-methyl cytosines and all of the other nucleobases of the modified oligonucleotide are unmodified nucleobases.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl moiety. In certain embodiments, the modified nucleobase is selected from: a 2-thiopyrimidine and a 5-propynepyrimidine.

3. Certain Internucleoside Linkage Motifs

In certain embodiments, oligonucleotides comprise modified and/or unmodified internucleoside linkages arranged along the oligonucleotide or region thereof in a defined pattern or motif. In certain embodiments, each internucleoside linking group is a phosphodiester internucleoside linkage (P═O). In certain embodiments, each internucleoside linking group of a modified oligonucleotide is a phosphorothioate internucleoside linkage (P═S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate, a (Sp) phosphorothioate, and a (Rp) phosphorothioate. In certain embodiments, the sugar motif of a modified oligonucleotide is a gapmer and the internucleoside linkages within the gap are all modified. In certain such embodiments, some or all of the internucleoside linkages in the wings are unmodified phosphodiester internucleoside linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester linkage is not a terminal internucleoside linkage, and the remaining internucleoside linkages are phosphorothioate internucleoside linkages. In certain such embodiments, all of the phosphorothioate linkages are stereorandom. In certain embodiments, all of the phosphorothioate linkages in the wings are (Sp) phosphorothioates, and the gap comprises at least one Sp, Sp, Rp motif. In certain embodiments, populations of modified oligonucleotides are enriched for modified oligonucleotides comprising such internucleoside linkage motifs.

C. Certain Lengths

It is possible to increase or decrease the length of an oligonucleotide without eliminating activity. For example, in Woolf et al. (Proc. Natl. Acad. Sci. USA 89:7305-7309, 1992), a series of oligonucleotides 13-25 nucleobases in length were tested for their ability to induce cleavage of a target RNA in an oocyte injection model. Oligonucleotides 25 nucleobases in length with 8 or 11 mismatch bases near the ends of the oligonucleotides were able to direct specific cleavage of the target RNA, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides

D. Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Unless otherwise indicated, all modifications are independent of nucleobase sequence.

E. Certain Populations of Modified Oligonucleotides

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population. In certain embodiments, the modified oligonucleotides of a chirally enriched population are enriched for f-D ribosyl sugar moieties, and all of the phosphorothioate internucleoside linkages are stereorandom. In certain embodiments, the modified oligonucleotides of a chirally enriched population are enriched for both p-D ribosyl sugar moieties and at least one, particular phosphorothioate internucleoside linkage in a particular stereochemical configuration.

F. Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 50%, at least 60%, at least 70%, at least 80%, at least 85%, at least 90%, at least 95%, or 100% complementary to the second oligonucleotide or nucleic acid, such as a target nucleic acid.

III. Oligomeric Duplexes

In certain embodiments, oligomeric compounds described herein comprise an oligonucleotide, having a nucleobase sequence complementary to that of a target nucleic acid. In certain embodiments, an oligomeric compound is paired with a second oligomeric compound to form an oligomeric duplex. Such oligomeric duplexes comprise a first oligomeric compound having a region complementary to a target nucleic acid and a second oligomeric compound having a region complementary to the first oligomeric compound. In certain embodiments, the first oligomeric compound of an oligomeric duplex comprises or consists essentially of a modified or unmodified oligonucleotide, a conjugate linker, a peptide extender, and a conjugate moiety. In certain embodiments, the second oligomeric compound of an oligomeric duplex comprises or consists essentially of a modified or unmodified oligonucleotide, a conjugate linker, a peptide extender, and a conjugate moiety. Either or both oligomeric compounds of an oligomeric duplex may comprise a conjugate linker, a peptide extender, and a conjugate moiety. In certain embodiments, the oligomeric compound is directly connected to the conjugate linker, the conjugate linker is directly connected to the peptide extender, and the peptide extender is directly connected to the conjugate moiety. The oligonucleotides of each oligomeric compound of an oligomeric duplex may include non-complementary overhanging nucleosides.

IV. Antisense Activity

In certain embodiments, oligomeric compounds and oligomeric duplexes are capable of hybridizing to a target nucleic acid, resulting in at least one antisense activity; such oligomeric compounds and oligomeric duplexes are antisense compounds. In certain embodiments, antisense compounds have antisense activity when they reduce or inhibit the amount or activity of a target nucleic acid by 25% or more in the standard cell assay. In certain embodiments, antisense compounds selectively affect one or more target nucleic acid. Such antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, antisense compounds described herein are sufficiently “DNA-like” to elicit RNase H activity. In certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi compounds. RNAi compounds may be double-stranded (siRNA) or single-stranded (ssRNA).

In certain embodiments, hybridization of an antisense compound to a target nucleic acid does not result in recruitment of a protein that cleaves that target nucleic acid. In certain embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain embodiments, hybridization of an antisense compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein and/or a phenotypic change in a cell or animal.

V. Certain Target Nucleic Acids

In certain embodiments, oligomeric compounds comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: a mature mRNA and a pre-mRNA, including intronic, exonic and untranslated regions. In certain embodiments, the target RNA is a mature mRNA. In certain embodiments, the target nucleic acid is a pre-mRNA. In certain such embodiments, the target region is entirely within an intron. In certain embodiments, the target region spans an intron/exon junction. In certain embodiments, the target region is at least 50% within an intron. In certain embodiments, the target nucleic acid is the RNA transcriptional product of a retrogene. In certain embodiments, the target nucleic acid is a non-coding RNA. In certain such embodiments, the target non-coding RNA is selected from: a long non-coding RNA, a short non-coding RNA, an intronic RNA molecule.

### Complementarity/Mismatches to the Target Nucleic Acid

It is possible to introduce mismatch bases without eliminating activity. For example, Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo. Maher and Dolnick (Nuc. Acid. Res. 16:3341-3358, 1988) tested a series of tandem 14 nucleobase oligonucleotides, and a 28 and 42 nucleobase oligonucleotides comprised of the sequence of two or three of the tandem oligonucleotides, respectively, for their ability to arrest translation of human DHFR in a rabbit reticulocyte assay. Each of the three 14 nucleobase oligonucleotides alone was able to inhibit translation, albeit at a more modest level than the 28 or 42 nucleobase oligonucleotides.

In certain embodiments, oligonucleotides are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a region that is 100% or fully complementary to a target nucleic acid. In certain embodiments, the region of full complementarity is from 6 to 20, 10 to 18, or 18 to 20 nucleobases in length.

In certain embodiments, oligonucleotides comprise one or more mismatched nucleobases relative to the target nucleic acid. In certain embodiments, antisense activity against the target is reduced by such mismatch, but activity against a non-target is reduced by a greater amount. Thus, in certain embodiments selectivity of the oligonucleotide is improved. In certain embodiments, the mismatch is specifically positioned within an oligonucleotide having a gapmer motif. In certain embodiments, the mismatch is at position 1, 2, 3, 4, 5, 6, 7, or 8 from the 5′-end of the gap region. In certain embodiments, the mismatch is at position 9, 8, 7, 6, 5, 4, 3, 2, 1 from the 3′-end of the gap region. In certain embodiments, the mismatch is at position 1, 2, 3, or 4 from the 5′-end of the wing region. In certain embodiments, the mismatch is at position 4, 3, 2, or 1 from the 3′-end of the wing region.

VI. Certain Pharmaceutical Compositions

In certain embodiments, pharmaceutical compositions described herein comprise one or more oligomeric compounds. In certain embodiments, the one or more oligomeric compounds each comprise a modified oligonucleotide. In certain embodiments, the pharmaceutical composition comprises a pharmaceutically acceptable diluent or carrier. In certain embodiments, a pharmaceutical composition comprises a sterile saline solution and one or more oligomeric compounds. In certain embodiments, a pharmaceutical composition consists or consists essentially of a sterile saline solution and one or more oligomeric compounds. In certain embodiments, the sterile saline is pharmaceutical grade saline. In certain embodiments, a pharmaceutical composition comprises one or more oligomeric compounds and sterile water. In certain embodiments, a pharmaceutical composition consists or consists essentially of one or more oligomeric compounds and sterile water. In certain embodiments, the sterile water is pharmaceutical grade water. In certain embodiments, the pharmaceutically acceptable diluent or carrier is distilled water for injection. In certain embodiments, a pharmaceutical composition comprises one or more oligomeric compound and phosphate-buffered saline (PBS). In certain embodiments, a pharmaceutical composition consists or consists essentially of one or more oligomeric compounds and PBS. In certain embodiments, the sterile PBS is pharmaceutical grade PBS. In certain embodiments, a pharmaceutical composition comprises one or more oligomeric compound and artificial cerebrospinal fluid. In certain embodiments, the sterile PBS is pharmaceutical grade PBS. In certain embodiments, a pharmaceutical composition consists or consists essentially of artificial cerebrospinal fluid. In certain embodiments, the artificial cerebrospinal fluid is pharmaceutical grade.

In certain embodiments, pharmaceutical compositions comprise one or more oligomeric compounds disclosed herein and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric compound disclosed herein encompass any pharmaceutically acceptable salts of the oligomeric compound, esters of the oligomeric compound, or salts of such esters. In certain embodiments, pharmaceutical compositions comprising oligomeric compounds comprising one or more oligonucleotide, upon administration to an animal, including a human, are capable of providing (directly or indirectly) the biologically active metabolite or residue thereof. Accordingly, for example, the disclosure is also drawn to pharmaceutically acceptable salts of oligomeric compounds, prodrugs, pharmaceutically acceptable salts of such prodrugs, and other bioequivalents. Suitable pharmaceutically acceptable salts include, but are not limited to, sodium and potassium salts. In certain embodiments, prodrugs comprise a conjugate moiety attached to an oligonucleotide, wherein the conjugate moiety is cleaved by endogenous nucleases within the body.

Lipid moieties have been used in nucleic acid therapies in a variety of methods. In certain such methods, the nucleic acid, such as an oligomeric compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions disclosed herein comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver oligomeric compounds described herein to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions disclosed herein are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain.

Under certain conditions, certain compounds disclosed herein act as acids. Although such compounds may be drawn or described in protonated (free acid) form, in ionized (anion) form, or ionized and in association with a cation (salt) form, aqueous solutions of such compounds exist in equilibrium among such forms. For example, a phosphate linkage of an oligonucleotide in aqueous solution exists in equilibrium among free acid, anion, and salt forms. Unless otherwise indicated, compounds described herein are intended to include all such forms. Moreover, certain oligonucleotides have several such linkages, each of which is in equilibrium. Thus, oligonucleotides in solution exist in an ensemble of forms at multiple positions all at equilibrium. The term “oligonucleotide” is intended to include all such forms. Drawn structures necessarily depict a single form. Nevertheless, unless otherwise indicated, such drawings are likewise intended to include corresponding forms. Herein, a structure depicting the free acid of a compound followed by the term “or salts thereof” expressly includes all such forms that may be fully or partially protonated/de-protonated/in association with a cation. In certain instances, one or more specific cation is identified.

In certain embodiments, oligomeric compounds disclosed herein are in aqueous solution with sodium. In certain embodiments, oligomeric compounds are in aqueous solution with potassium. In certain embodiments, oligomeric compounds are in PBS. In certain embodiments, oligomeric compounds are in water. In certain such embodiments, the pH of the solution is adjusted with NaOH and/or HCl to achieve a desired pH.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif And, for example, where a particular high-affinity modification appears at a particular position, other high-affinity modifications at the same position are considered suitable, unless otherwise indicated.

### Example 1: Design, Synthesis and Uptake of a Modified Oligonucleotide Conjugated to Cy3 and Neurotensin Peptide

Compound No. 1016003 (1), a 3′-Cy3 and 5′-hexylamino modified uniform phosphodiester 2′-O-(2-methoxyethyl RNA) was designed, the details of which are as indicated in the table below. Compound No. 1016003 is a uniform MOE compound, wherein every sugar moiety throughout the modified oligonucleotide is a 2′-MOE sugar moiety. The internucleoside linkages throughout the modified oligonucleotide are phosphodiester linkages, and are denoted by “o” in the table below. All cytosine residues are 5-methylcytosines. 1016003 is 100% complementary to human MALAT RNA SEQ ID NO: 1 (GENBANK Accession No. NT_033903.7 truncated from nucleotides 10569000 to 10582000) at positions 6925 to 6944.

TABLE 1
Design of modified oligonucleotides

| Com- | Sequence | SEQ | | | |
|---|---|---|---|---|---|
| pound | (5′ to | ID | 5′- | 3′- | 5′- |
| No. | 3′) | NO | conjugate | conjugate | linker |
| 1016003 | CCAGGCT | 2 | (o)-6- | Cy3 | phospho- |
| | GGTTATG | | amino- | (thioate) | diester |
| | ACTCAG | | hexanol | | |

As shown in Scheme 1a below, Compound No. 1016003 (1) was treated with BCN—N-hydroxysuccinimide ester (NHS) reagent (2) to yield BCN (bicyclo[6.1.0]nonyne) containing modified oligonucleotide (3). “ASO” in Scheme 1 below refers to the sequence (from 3′ to 5′): GACTCAGTATTGGTCGGACC (SEQ ID NO: 16). To make N-terminus of neurotensin suitable for azido modification, the pyroglutamic acid residue ([pE]) at the N-terminus of neurotensin peptide ([pE]-LYENKPRRPYIL, SEQ ID NO: 72) was replaced with a lysine residue. Next, an 2-azido-acetyl group was inserted at the N-terminus lysine to yield a modified neurotensin 4 (KNT, XLYENKPRRPYIL, SEQ ID NO: 3, wherein X is 2-azido-acetyl lysine, Scheme 1a). The azido-acetyl group is attached to the side chain amine as shown below:

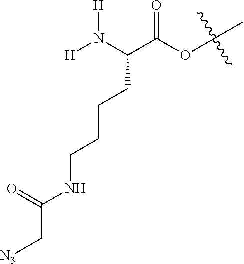 [SMILES: *OC(=O)[C@@H](N)CCCCNC(=O)CN=[N+]=[N-]]

The final coupling reaction was carried out in DMSO-sodium tetraborate buffer (0.1 M, pH 8.5) mixture at ambient temperature and the final product, Compound No. 1162754 (5) was purified by strong anion-exchange chromatography and desalted by HPLC on a reverse phase column. Compound No. 1162754 details are as indicated in Table 2. Compound No. 1162754 is a uniform MOE compound, wherein every sugar moiety throughout the modified oligonucleotide is a 2′-MOE sugar moiety. The internucleoside linkages throughout the modified oligonucleotide are phosphodiester linkages, and are denoted by “o” in the table below. All cytosine residues are 5-methylcytosines. 1162754 is 100% complementary to human MALAT RNA SEQ ID NO: 1 (GENBANK Accession No. NT_033903.7 truncated from nucleotides 10569000 to 10582000) at positions 6925 to 6944.

TABLE 2
Design of modified oligonucleotides

| | Se- | | | | | |
|---|---|---|---|---|---|---|
| Com- | quence | SEQ | 5′- | SEQ | 3′- | |
| pound | (5′ to | ID | conju- | ID | conju- | 5′- |
| No. | 3′) | NO | gate | NO | gate | linker |
| 1162754 | CCAGG | 2 | dKLYEN | 3 | Cy3 | (o)-6- |
| | CTGGT | | KPRRPY | | (thioate) | aminohexanol- |
| | TATGA | | IL | | | 1- |
| | CTCAG | | | | | carboxymethyl |
| | | | | | | [triazoloBCN1] |
| | | | | | | carbamate |

As positive control, a Cy3-labeled KNT, XLYENKPRRPYIL (SEQ ID NO: 3) wherein X is 2-azido-acetyl lysine, Compound No. 17-AH-1493 (7) (Scheme 1b) was synthesized using peptide (4) and a previously reported Cy3-BCN reagent (6) using the same synthetic method described above (Scheme 1b).

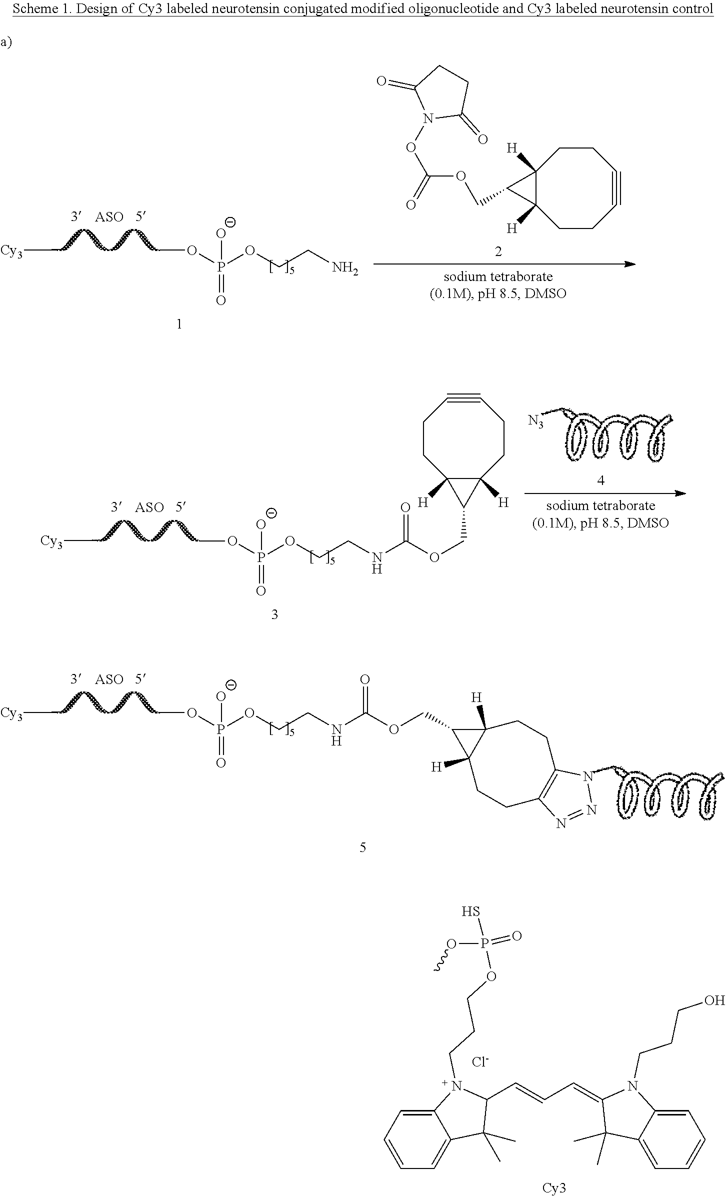

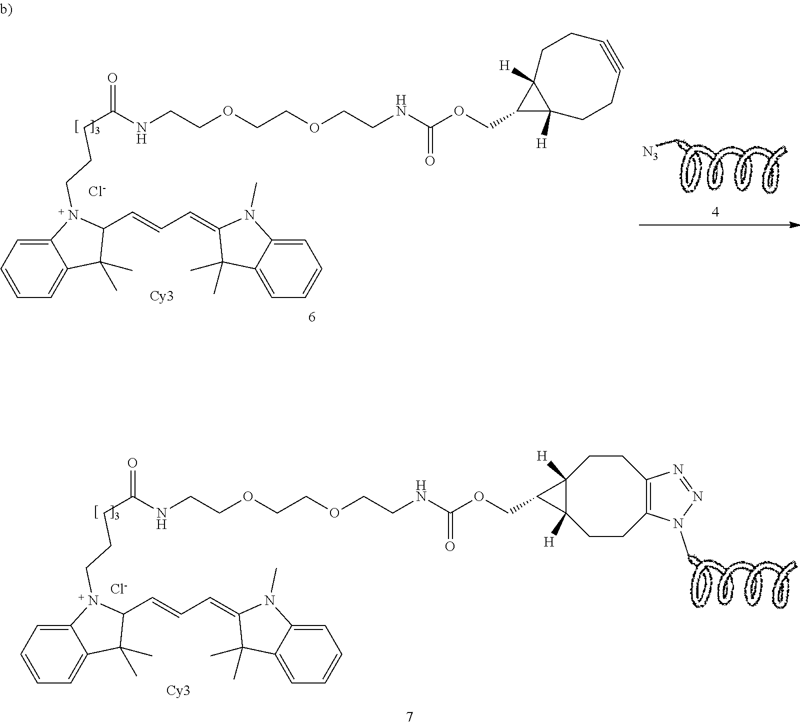

HEK cells stably expressing human sortilin (HEK-SORT1 cells) were generated by infecting HEK 293 cells (ATCC) with lentivirus produced by transfection of 293T cells with pLVX-IRES-Puro (Clontech Laboratories Inc., Mountainview, CA) harboring the SORT1 insert.

HEK-SORT1 cells were seeded in 24-well plates (at approximately 150,000 cells/well) and allowed to attach and equilibrate for at least 16 hours. The indicated Cy3-lableled compounds (U.S. Pat. Nos. 1,016,003 and 1,162,754), were added to wells at the concentrations indicated in table 3 below. In addition, 17-AH-1493, which consists of the neurotensin peptide motif XLYENKPRRPYIL (SEQ ID NO: 3), wherein in X is D-Lysine, conjugated to Cy3 at its N-terminus, was added as a comparator compound. Two plates were prepared for each treatment condition, one serving as 4° C. no internalization control that was kept on ice during incubation, while the second plate was incubated at 37° C. to allow for energy-dependent internalization. Following the incubation period all plates were placed on ice and washed 3 times with ice-cold PBS/3% BSA/2 mM EDTA then lifted with trypsin, diluted into wash buffer, and placed into flow cytometry tubes on ice. Cells were then analyzed by flow cytometry for mean fluorescence intensity using a BD FACS Calibur flow cytometer (BD Biosciences, San Jose, CA, USA). Internalized modified oligonucleotide was expressed as the difference between the corresponding 4° C. and 37° C. mean fluorescence intensities.

As seen in the Table 3 below, conjugation of the modified oligonucleotide to the neurotensin peptide did not improve uptake of modified oligonucleotide compared to the peptide-alone control.

TABLE 3
Effect of peptide conjugation on modified oligonucleotide uptake

| Concentration | Mean Fluorescence Intensity (MFI) | | |
|---|---|---|---|
| (nM) | 17-AH-1493 | 1016003 | 1162754 |
| 3000 | 1387 | 291 | 690 |
| 1000 | 1229 | 154 | 293 |
| 333 | 1126 | 126 | 171 |
| 111 | 928 | 141 | 71 |
| 37 | 362 | 49 | 54 |
| 12 | 126 | 45 | 43 |

### Example 2: Synthesis of Neurotensin Peptide-Morpholino Conjugate and Comparison to Neurotensin Peptide Conjugated Modified Oligonucleotide

To determine whether the conjugation of neurotensin to a charge neutral morpholino could minimize undesired electrostatic interactions that may be occurring due to positively charged residues within the neurotensin peptide, phosphorodiamidate morpholino oligomers (PMOs) were used to synthesize a morpholino-neurotensin conjugate. The PMO comprises a 3′-azido modification and a 5′-modification as shown below:

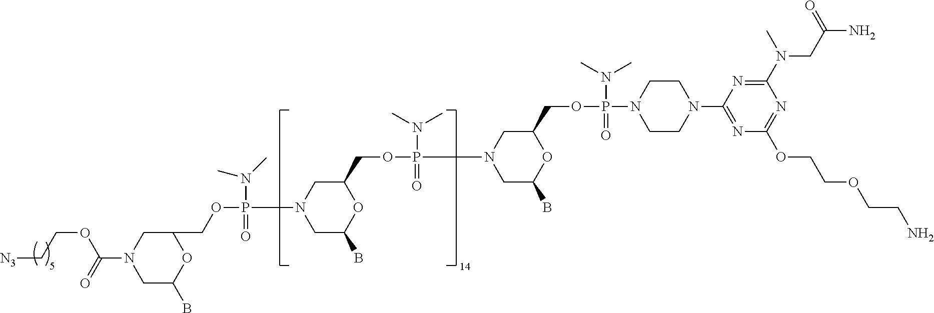

The details of the synthesis process are outlined in Scheme 2 below. In brief, the 3′-azido modified phosphoramidite morpholino oligomer 8 was reacted with Cy3-BCN reagent 6 in DMSO to generate Cy3 labeled Morpholino oligomer 9 (Scheme 2). Compound 9 was then reacted with reagent 2 in DMSO-sodium tetraborate buffer (0.1 M, pH 8.5) mixture to produce BCN-modified Morpholino oligomer 10 (designated as Compound No. 18-HD1402 herein). Finally, compound 10 was reacted with modified neurotensin peptide 4 to yield the desired Morpholino oligomer-neurotensin conjugate 11 (designated as Compound No. 18-AJ9998 herein).

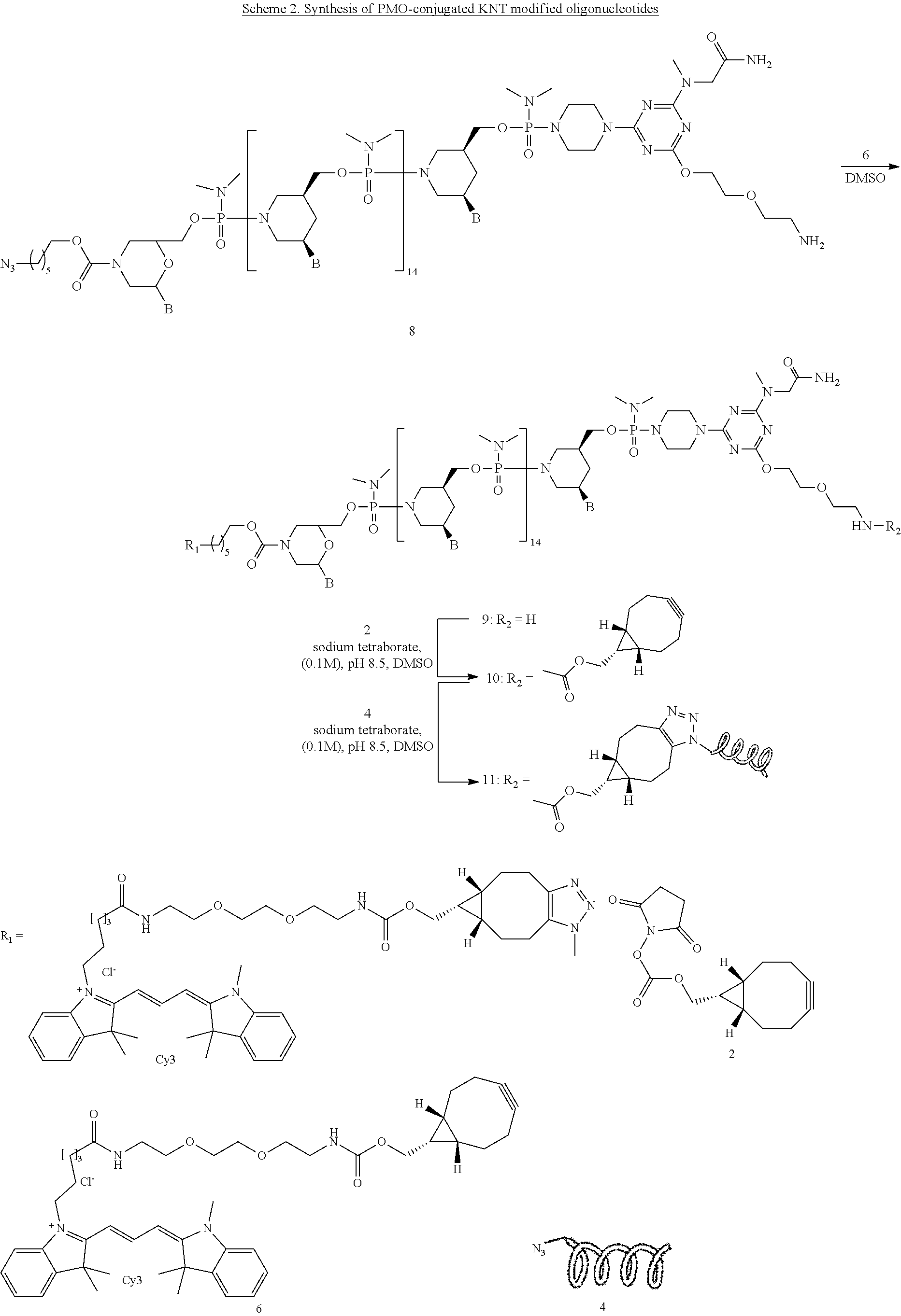

HEK-SORT1 cells were seeded in 24-well plates (at ˜150,000 cells/well) and allowed to attach and equilibrate for at least 16 hours. The indicated Cy3-lableled compounds (18-AJ9998, 18-HD1402, 17-AH-1493 and 1162754 described herein above), were added to wells at the concentrations indicated in Table 4 below. Two plates were prepared for each treatment condition, one serving as 4° C. no internalization control that was kept on ice during incubation, while the second plate was incubated at 37° C. to allow for energy-dependent internalization. Following the incubation period all plates were placed on ice and washed 3 times with ice-cold PBS/3% BSA/2 mM EDTA then lifted with trypsin, diluted into wash buffer, and placed into flow cytometry tubes on ice. Cells were then analyzed by flow cytometry for mean fluorescence intensity using a BD FACS Calibur flow cytometer (BD Biosciences, San Jose, CA, USA). Internalized modified oligonucleotide was expressed as the difference between the corresponding 4° C. and 37° C. mean fluorescence intensities.

The morpholino is charge neutral. The peptide-morpholino conjugate 18-HD1402 shows comparable uptake to peptide alone (17-AH-1493). The data below suggests that the charge of the modified oligonucleotide interferes with binding of neurotensin to the receptor.

TABLE 4
Effect of charge on modified oligonucleotide uptake

| Concentration | Mean Fluorescence Intensity (MFI) | | | |
|---|---|---|---|---|
| (nM) | 17-AH-1493 | 18-AJ9998 | 18-HD1402 | 1162754 |
| 3000 | 1365 | 631 | 1184 | 427 |
| 1000 | 866 | 267 | 1011 | 192 |
| 333 | 589 | 138 | 786 | 123 |
| 111 | 326 | 86 | 532 | 85 |
| 37 | 139 | 75 | 292 | 72 |
| 12 | 100 | 70 | 163 | 67 |

### Example 3: Effect of Modified Oligonucleotide Length on Neurotensin Peptide Conjugated Modified Oligonucleotide Uptake into HEK-SORT1 Cells

Modified oligonucleotides of varying lengths conjugated to neurotensin peptide were designed per description in Table 5 and synthesized according to the method shown in Scheme 1 herein above, wherein the sequence of the “ASO” in Scheme 1 was replaced with the sequence (from 3′ to 5′): GACTCAGTATTGGTCG (SEQ ID NO: 9) to synthesize Compound No. 1247096, wherein the sequence of the “ASO” in Scheme 1 was replaced with the sequence (from 3′ to 5′): GACTCAGTATTG (SEQ ID NO: 10) to synthesize Compound No. 1247097, wherein the sequence of the “ASO” in Scheme 1 was replaced with the sequence (from 3′ to 5′): GACTCAGT (SEQ ID NO: 11) to synthesize Compound No. 1247098, and wherein the sequence of the “ASO” in Scheme 1 was replaced with the sequence (from 3′ to 5′): GACT (SEQ ID NO: 12) to synthesize Compound No. 1247099.

All the modified oligonucleotides in the table below are uniform MOE oligonucleotides, wherein every sugar moiety throughout the modified oligonucleotides is a 2′-MOE sugar moiety. The internucleoside linkages throughout the modified oligonucleotides in the table below are phosphodiester linkages, and are denoted by “o” in the table below. All cytosine residues are 5-methylcytosines. All the compounds are labeled with Cy3 at the 3′-end.

The N-terminal end of the neurotensin peptide motif is conjugated to the 5′-end of the oligonucleotide through linkers indicated in Table 5. The attachment point of the peptide to the linker is underlined in the table below (Table 5). The compounds indicated in table 5 are 100% complementary to human MALAT RNA SEQ ID NO: 1 wherein the stop site of all the compounds in table 5 is position 6944 of SEQ ID NO: 1.

TABLE 5
Design of modified oligonucleotides

| | Se- | | | | | |
|---|---|---|---|---|---|---|
| Com- | quence | SEQ | 5′- | SEQ | 3′- | |
| pound | (5′ to | ID | conju- | ID | conju- | 5′- |
| No. | 3′) | NO | gate | NO | gate | linker |
| 1247096 | GCTGG | 9 | dKLY | 3 | Cy3 | (o)-6- |
| | TTATG | | ENKP | | (thioate) | aminohexanol- |
| | ACTCA | | RRPY | | | 1- |
| | G | | IL | | | carboxymethyl |
| | | | | | | [triazoloBCN1] |
| | | | | | | carbamate |
| 1247097 | GTTA | 10 | dKLY | 3 | Cy3 | (o)-6- |
| | TGAC | | ENKP | | (thioate) | aminohexanol- |
| | TCAG | | RRPY | | | 1- |
| | | | IL | | | carboxymethyl |
| | | | | | | [triazoloBCN1] |
| | | | | | | carbamate |
| 1247098 | TGAC | 11 | dKLY | 3 | Cy3 | (o)-6- |
| | TCAG | | ENKP | | (thioate) | aminohexanol- |
| | | | RRPY | | | 1- |
| | | | IL | | | carboxymethyl |
| | | | | | | [triazoloBCN1] |
| | | | | | | carbamate |
| 1247099 | TCAG | 12 | dKLY | 3 | Cy3 | (o)-6- |
| | | | ENKP | | (thioate) | aminohexanol- |
| | | | RRPY | | | 1- |
| | | | IL | | | carboxymethyl |
| | | | | | | [triazoloBCN1] |
| | | | | | | carbamate |

HEK-SORT1 cells were seeded in 24-well plates (at ˜150,000 cells/well) and allowed to attach and equilibrate for at least 16 hours. The indicated Cy3-lableled compounds (U.S. Pat. Nos. 1,162,754, 1,247,096, 1,247,097, 1,247,098, and 1,247,099 described herein above), were added to wells at the concentrations indicated in Table 6 below. Two plates were prepared for each treatment condition, one serving as 4° C. no internalization control that was kept on ice during incubation, while the second plate was incubated at 37° C. to allow for energy-dependent internalization. Following the incubation period all plates were placed on ice and washed 3 times with ice-cold PBS/3% BSA/2 mM EDTA then lifted with trypsin, diluted into wash buffer, and placed into flow cytometry tubes on ice. Cells were then analyzed by flow cytometry for mean fluorescence intensity using a BD FACS Calibur flow cytometer (BD Biosciences, San Jose, CA, USA). Internalized modified oligonucleotide was expressed as the difference between the corresponding 4° C. and 37° C. mean fluorescence intensities.

The data indicate that the oligonucleotide length affects uptake of the neurotensin-conjugated modified oligonucleotides.

TABLE 6
Effect of oligonucleotide length on Neurotensin
peptide conjugated oligonucleotide uptake

| Concen- | | | | | |
|---|---|---|---|---|---|
| tration | Mean Fluorescence Intensity (MFI) | | | | |
| (nM) | 1162754 | 1247096 | 1247097 | 1247098 | 1247099 |
| 1000 | 370 | 2230 | 2575 | 2170 | 2146 |
| 333 | 205 | 937 | 1354 | 1401 | 1681 |
| 111 | 101 | 446 | 483 | 590 | 973 |
| 37 | 66 | 167 | 169 | 226 | 544 |
| 12 | 54 | 83 | 78 | 110 | 223 |

### Example 4: Effect of Peptide Charge on Uptake of Modified Oligonucleotides Conjugated to Neurotensin Peptides

The effect of peptide conjugate charge on binding was determined by designing three different peptide motifs and conjugating them to modified oligonucleotides as described in the table below. The peptides include XLYENKPKRPYIL (SEQ ID NO: 73) and XLYENKPRKPYIL (SEQ ID NO: 74), wherein X is lysine, and wherein the Arg8 or Arg9 are replaced with a lysine. The third peptide motif tested was KLYENKPKKPYIL (SEQ ID NO: 75), wherein two lysines were inserted at positions 8 and 9. The conjugated modified oligonucleotides were synthesized according to the method shown in Scheme 1 herein above, wherein peptide 4 having an amino acid sequence of XLYENKPRRPYIL (SEQ ID NO: 3), wherein X is 2-azido-acetyl lysine, in Scheme 1 was replaced with the peptide sequence 2-azido-acetyl-KLYENKPKKPYIL (SEQ ID NO: 75) to generate Compound No. 1270697, wherein peptide 4 having an amino acid sequence of 2-azido-acetyl-XLYENKPRRPYIL (SEQ ID NO: 3), wherein X is 2-azido-acetyl lysine, in Scheme 1 was replaced with the peptide sequence XLYENKPKRPYIL (SEQ ID NO: 73), wherein X is a 2-azido-acetyl lysine, to generate Compound No. 1270698, and wherein the peptide 4 having an amino acid sequence of XLYENKPRRPYIL (SEQ ID NO: 3), wherein X is azido-acetyl lysine in Scheme 1 was replaced with the peptide sequence XLYENKPRKPYIL (SEQ ID NO: 74), wherein X is a 2-azido-acetyl lysine, to generate Compound No. 1270696.

All the modified oligonucleotides in the table below are uniform MOE oligonucleotides, wherein every sugar moiety throughout the modified oligonucleotide is a 2′-MOE sugar moiety. The internucleoside linkages throughout the modified oligonucleotides in the table below are phosphodiester linkages, and are denoted by “o” in the table below. All cytosine residues in the modified oligonucleotides in the table below are 5-methylcytosines. All the compounds are labeled with Cy3 at the 3′-end. The modified oligonucleotides are 100% complementary to human MALAT RNA SEQ ID NO: 1 (GENBANK Accession No. NT_033903.7 truncated from nucleotides 10569000 to 10582000) at positions 6925 to 6944.

TABLE 7
Design of modified oligonucleotides

| | Se- | | | | | |
|---|---|---|---|---|---|---|
| Com- | quence | SEQ | 5′- | SEQ | 3′- | |
| pound | (5′ to | ID | conju- | ID | conju- | 5′- |
| No. | 3′) | NO | gate | NO | gate | linker |
| 1270697 | CCAG | 2 | dKLY | 75 | Cy3 | (o)-6-amino |
| | GCTG | | ENKP | | (thioate) | hexanol- |
| | GTTA | | RRPY | | | 1- |
| | TGAC | | IL | | | carboxymethyl |
| | TCAG | | | | | [triazoloBCN1] |
| | | | | | | carbamate |
| 1270698 | CCAG | 2 | dKLY | 73 | Cy3 | (o)-6-amino |
| | GCTG | | ENKP | | (thioate) | hexanol- |
| | GTTA | | RRPY | | | 1- |
| | TGAC | | IL | | | carboxymethyl |
| | TCAG | | | | | [triazoloBCN1] |
| | | | | | | carbamate |
| 1270696 | CCAG | 2 | dKLY | 74 | Cy3 | (o)-6-amino |
| | GCTG | | ENKP | | (thioate) | hexanol- |
| | GTTA | | RRPY | | | 1- |
| | TGAC | | IL | | | carboxymethyl |
| | TCAG | | | | | [triazoloBCN1] |

HEK-SORT1 cells were seeded in 24-well plates (at ˜150,000 cells/well) and allowed to attach and equilibrate for at least 16 hours. The indicated Cy3-lableled compounds (1162754, 17-AH-1493, 1270696, 1270697 and 1270698 described herein above), were added to wells at the concentrations indicated in the table below. Two plates were prepared for each treatment condition, one serving as 4° C. no internalization control that was kept on ice during incubation, while the second plate was incubated at 37° C. to allow for energy-dependent internalization. Following the incubation period all plates were placed on ice and washed 3 times with ice-cold PBS/3% BSA/2 mM EDTA then lifted with trypsin, diluted into wash buffer, and placed into flow cytometry tubes on ice. Cells were then analyzed by flow cytometry for mean fluorescence intensity using a BD FACS Calibur flow cytometer (BD Biosciences, San Jose, CA, USA). Internalized modified oligonucleotide was expressed as the difference between the corresponding 4° C. and 37° C. mean fluorescence intensities.

TABLE 8
Effect of extended non-peptide spacer
on modified oligonucleotide uptake

| Concen- | | | | | |
|---|---|---|---|---|---|
| tration | Mean Fluorescence Intensity (MFI) | | | | |
| (nM) | 1162754 | 1270697 | 1270698 | 1270696 | 17-AH-1493 |
| 1000 | 371 | 474 | 394 | 1100 | 1102 |
| 333 | 143 | 185 | 155 | 312 | 869 |
| 111 | 72 | 86 | 78 | 138 | 396 |
| 37 | 47 | 51 | 48 | 61 | 163 |
| 12 | 38 | 39 | 35 | 43 | 49 |

### Example 5: Effect of Non-Peptide Spacers on Uptake of Modified Oligonucleotides Conjugated to Neurotensin Peptide

Three compounds with non-peptidic spacers were synthesized to evaluate the effect of a longer spacer on the uptake of modified oligonucleotide conjugated to a neurotensin peptide. Two of these modified oligonucleotide conjugates have polyethylene glycol (PEG) spacers of n=10 and n=19 atoms (Compound Nos. 1186041 and 1186505 respectively). The third modified oligonucleotide (Compound No. 1186506) is a hydrocarbon chain interrupted by a disulfide cleavable bond. All three spacers were introduced during the solid-phase synthesis of the modified oligonucleotide using commercially available phosphoramidite reagents. The neurotensin conjugated modified oligonucleotides with extended spacers were synthesized according to the method shown in Scheme 1 herein above, wherein Compound No. 1016003 (1) was replaced by the structure below. “ASO” in the figure below refers to the sequence (from 3′ to 5′): GACTCAGTATTGGTCGGACC, SEQ ID NO: 16. The Spacer in the structure below refers to either Spacer 1 (to generate Compounds No. 1186041), Spacer 2 (to generate Compound No. 1186505), or Spacer 3 to generate Compound No. 1186506.

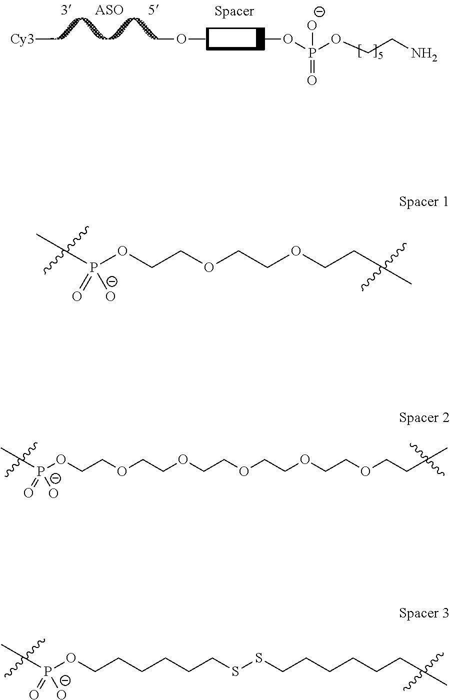

All the compounds described in Table 9 below, are 100% complementary to human MALAT RNA SEQ ID NO: 1 at start site 6925 to stop site 6944. All the modified oligonucleotides in the table below are uniform MOE compounds, wherein every sugar moiety throughout the modified oligonucleotide is a 2′-MOE sugar moiety. The internucleoside linkages throughout the modified oligonucleotides in the table below are phosphodiester linkages, and are denoted by “o” in the table below. All cytosine residues are 5-methylcytosines. All the compounds are labeled with Cy3 at the 3′-end.

TABLE 9
Design of modified oligonucleotides

| Compound | Sequence | SEQ | 5′- | SEQ | 3′- | |
|---|---|---|---|---|---|---|
| No. | (5′ to 3′) | ID NO | conjugate | ID NO | conjugate | 5′-linker |
| 1186041 | CCAGGCTG | 2 | dKLY | 3 | Cy3 (thioate) | (o)-TEG (2,2′-(ethane-1,2- |
| | GTTATGAC | | ENKP | | | diylbis(oxy))bis(ehtan-1-ol)- |
| | TCAG | | RRPY | | | (o)-6-aminohexanol-1- |
| | | | IL | | | carboxymethyl[triazoloBCN1] |
| | | | | | | carbamate |
| 1186505 | CCAGGCTG | 2 | dKLY | 3 | Cy3 (thioate) | (o)-HEG (hexaethylene glycol)- |
| | GTTATGAC | | ENKP | | | (o)-6-aminohexanol-1- |
| | TCAG | | RRPY | | | carboxymethyl [triazoloBCN1] |
| | | | IL | | | carbamate |
| 1186506 | CCAGGCTG | 2 | dKLY | 3 | Cy3 (thioate) | (o)-C6-S-S-C6 (6- |
| | GTTATGAC | | ENKP | | | mercaptohexanol-6- |
| | TCAG | | RRPY | | | mercaptohexanol)-(o)-6- |
| | | | IL | | | aminohexanol-1- |
| | | | | | | carboxymethyl [triazoloBCN1] |
| | | | | | | carbamate |

HEK-SORT1 cells were seeded in 24-well plates (at ˜150,000 cells/well) and allowed to attach and equilibrate for at least 16 hours. The indicated Cy3-lableled compounds (U.S. Pat. Nos. 1,162,754, 1,186,041, 1,186,505 and 1,186,506 described herein above), were added to wells at the concentrations indicated in Table 10 below. Two plates were prepared for each treatment condition, one serving as 4° C. no internalization control that was kept on ice during incubation, while the second plate was incubated at 37° C. to allow for energy-dependent internalization. Following the incubation period all plates were placed on ice and washed 3 times with ice-cold PBS/3% BSA/2 mM EDTA then lifted with trypsin, diluted into wash buffer, and placed into flow cytometry tubes on ice. Cells were then analyzed by flow cytometry for mean fluorescence intensity using a BD FACS Calibur flow cytometer (BD Biosciences, San Jose, CA, USA). Internalized modified oligonucleotide was expressed as the difference between the corresponding 4° C. and 37° C. mean fluorescence intensities.

TABLE 10
Effect of extended non-peptide spacer
on modified oligonucleotide uptake

| Concen- | | | | | |
|---|---|---|---|---|---|
| tration | Mean Fluorescence Intensity (MFI) | | | | |
| (nM) | 17-AH-1493 | 1162754 | 1186041 | 1186505 | 1186506 |
| 1000 | 1229 | 293 | 346 | 537 | 735 |
| 333 | 1126 | 171 | 170 | 245 | 247 |
| 111 | 928 | 71 | 76 | 79 | 98 |
| 37 | 362 | 54 | 52 | 52 | 55 |
| 12 | 126 | 43 | 41 | 40 | 44 |

### Example 6: Synthesis and Uptake of Sortilin Propeptide Conjugated Modified Oligonucleotides

In addition to neurotensin, other sortilin ligands were tested for their ability to enhance modified oligonucleotide uptake into cells. The sortilin propeptide is designated as Propeptide 31 (CQDRLDAPPPPAAPLPRWSGPIGVSWGLRAAAAGGAFPRGGRWRR, SEQ ID NO: 76) in Scheme 3 below. In addition to the full length propeptide, a truncated 28-amino acid form of this peptide, was also tested. The truncated peptide is designated as Propeptide 30 (CQDRLDAPPPPAAPLPRWSGPIGVSWGLR, SEQ ID NO: 77) in Scheme 3 below. “ASO” in Scheme 3 below refers to the sequence (from 3′ to 5′): GACTCAGTATTGGTCGGACC, SEQ ID NO: 16. To synthesize the conjugated modified oligonucleotides, 5′-hexylamino modified oligonucleotide (Compound No. 1016003, 1) at 1 mol was dissolved in 0.1 M sodium phosphate buffer (0.6 mL/μmol, pH 7.2). To this, N-succinimidyl 3-maleimidopropionate 28 (5 mol, Scheme 3) in 0.1 mL/μmol DMSO was added. The reaction mixture was stirred at room temperature for 2 h. Water (5 mL/μmol) was added and the resulting solution was purified by HPLC on a strong anion exchange column (GE Healthcare Life Sciences SOURCE 30Q) using a linear gradient of buffer A (100 mM NH₄OAc in water containing 30%) to buffer B (11.5M NaBr), and desalted by HPLC on a reverse phase column. To a solution of 1.0 μmol 5′-(3-maleimidyl)propionyl-hexylamino modified oligonucleotide (Compound 29) dissolved in degassed 0.1M sodium phosphate buffer (1.0 mL/μmol, pH 7.2), a solution of the propeptide (1.3-2 μmol) containing a free cysteine in degassed DMF (0.25 mL/μmol) was added. Both peptides were functionalized with a cysteine moiety at the C-terminus (30 and 31, Scheme 3) and conjugated to the maleimide-functionalized modified oligonucleotide (29) according to the Scheme 3 to yield 32 (designated herein as Compound No. 1215810, with the propeptide sequence CQDRLDAPPPPAAPLPRWSGPIGVSWGLR, SEQ ID NO: 77) and 33 (designated herein as Compound No. 1215811, with the propeptide sequence CQDRLDAPPPPAAPLPRWSGPIGVSWGLRAAAAGGAFPRGGRWRR, SEQ ID NO: 76). The reaction mixture was stirred at room temperature for 2-3 h. Progress of the reaction was monitored by LC-MS analysis. After the completion of reaction as assessed by LC-MS analysis, reaction was diluted with water 5 mL/μmol) and purified by HPLC on a strong anion exchange column (GE Healthcare Life Sciences SOURCE 30Q) using a linear gradient of buffer A (100 mM NH₄OAc in water containing 30%) to buffer B (11.5M NaBr), and desalted by HPLC on a reverse phase column.

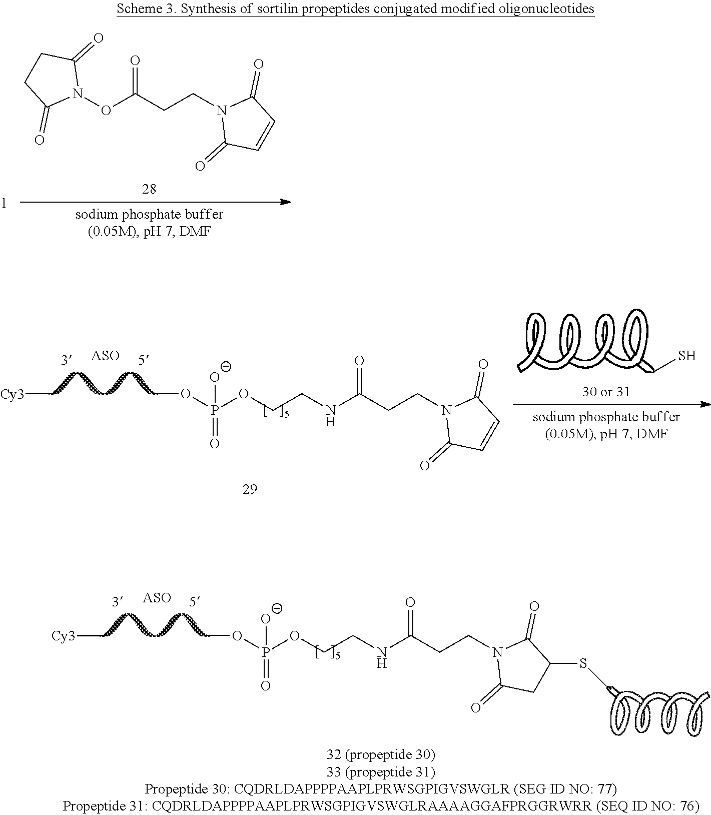

HEK-SORT1 cells were seeded in 24-well plates (at ˜150,000 cells/well) and allowed to attach and equilibrate for at least 16 hours. The indicated Cy3-lableled compounds (U.S. Pat. Nos. 1,215,810 and 1,215,811 described herein above), were added to wells at the concentrations indicated in table below. Two plates were prepared for each treatment condition, one serving as 4° C. no internalization control that was kept on ice during incubation, while the second plate was incubated at 37° C. to allow for energy-dependent internalization. Following the incubation period all plates were placed on ice and washed 3 times with ice-cold PBS/3% BSA/2 mM EDTA then lifted with trypsin, diluted into wash buffer, and placed into flow cytometry tubes on ice. Cells were then analyzed by flow cytometry for mean fluorescence intensity using a BD FACS Calibur flow cytometer (BD Biosciences, San Jose, CA, USA). Internalized modified oligonucleotide was expressed as the difference between the corresponding 4° C. and 37° C. mean fluorescence intensities.

TABLE 11
Effect of charge on modified oligonucleotide uptake

| | |
|---|---|
| Concentration | Mean Fluorescence Intensity (MFI) |

| (nM) | 1215810 | 1215811 |
|---|---|---|
| 882 | 432 | 1829 |
| 260 | 154 | 696 |
| 76 | 70 | 204 |
| 22 | 48 | 78 |
| 7 | 41 | 51 |

### Example 7: Effect of Extended Neurotensin Peptide Conjugated Modified Oligonucleotides on Uptake into HEK-SORT1 Cells

Synthesis and Uptake of Uniform 2′-MOE Modified Oligonucleotides with Extended Neurotensin Peptide Conjugates

A short sequence of 9 amino acids (PPPAGSSPG (SEQ ID NO: 38)) was inserted into the N-terminus of neurotensin to make a 23 amino acid peptide named extended neurotensin (eNT, 25, [azido-acetyl-KJPPPAGSSPGLYENKPRRPYIL (SEQ ID NO:5)). To generate a modified oligonucleotide conjugate containing the extended neurotensin, synthesis was carried out according to Scheme 1a described herein above, wherein KNT (4) was replaced by eNT (25, XPPPAGSSPGLYENKPRRPYIL (SEQ ID NO: 5), wherein X is azido-acetyl lysine) to generate Compound No. 1162756. Similarly, a control compound 17-AH-1492 was generated according to Scheme 1b described herein above, wherein KNT (4) was replaced by eNT (25, azido-acetyl KPPPAGSSPGLYENKPRRPYIL (SEQ ID NO: 5). The structures of the eNT, 17-AH-1492 and Compound No. 1162756 are as indicated below.

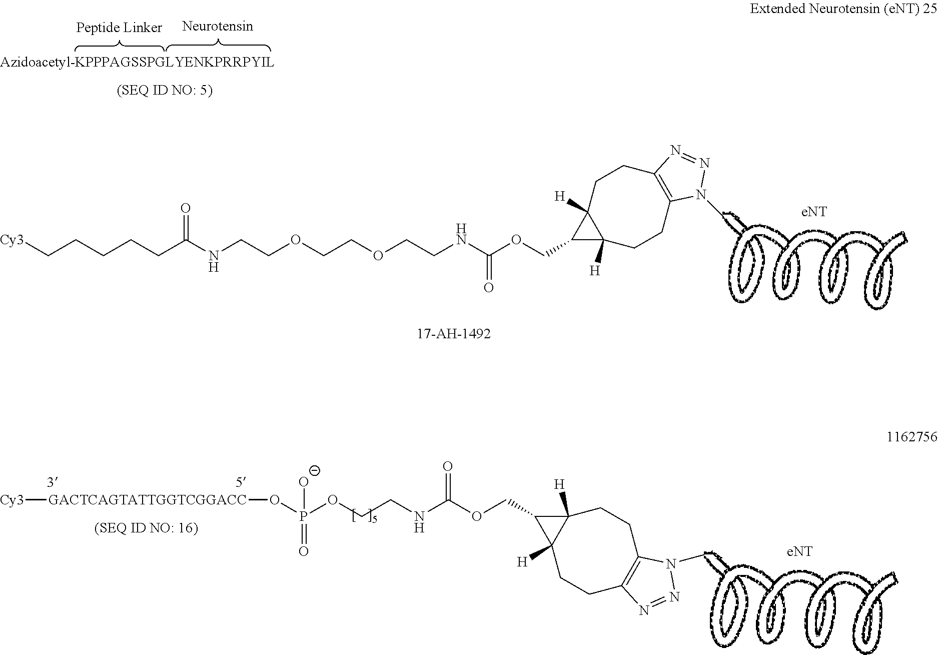

Separately, instead of a terminal lysine reside, modified oligonucleotides were designed with a second extended neurotensin peptide that contains an “SO5P” or “5-oxoproline” residue instead of lysine. The sequence of the alternate extended neurotensin peptide is [SO5P]-LYEN[azido-acetyl-K]PRRPYIL (SEQ ID NO: 4). To generate a modified oligonucleotide conjugate containing the [SO5P]-LYEN[azido-acetyl-K]PRRPYIL peptide (SEQ ID NO: 4), synthesis was carried out according to Scheme 1a described herein above, wherein KNT (4) was replaced by [SO5P]-LYEN[azido-acetyl-K]PRRPYIL (SEQ ID NO: 4) to generate Compound No. 1091165. Here, the reactive azido acetyl group is on the side chain of an internal lysine, as indicated. To generate a modified oligonucleotide conjugate containing the XAPSGPSPGLYENKPRRPYIL peptide (SEQ ID NO: 6, wherein X is azido-acetyl lysine), synthesis was carried out according to Scheme 1a described herein above, wherein KNT (4) was replaced by XPAPSGPSPGLYENKPRRPYIL (SEQ ID NO: 6, wherein X azido-acetyl lysine) to generate Compound No. 1252443. To generate a modified oligonucleotide conjugate containing a peptide having the amino acid sequence of XAGSIKPPPAGSSPGLYENKPRRPYIL (SEQ ID NO: 7, wherein X azido-acetyl lysine), synthesis was carried out according to Scheme 1a described herein above, wherein KNT (4) was replaced by XAGSIKPPPAGSSPGLYENKPRRPYIL (SEQ ID NO: 7, wherein X is azido-acetyl lysine) to generate Compound No. 1252444. To generate a modified oligonucleotide conjugate containing a peptide having the amino acid sequence of XAGMSGASAGLYENKPRRPYIL (SEQ ID NO: 8, wherein X azido-acetyl lysine), synthesis was cardied out according to Scheme 1a described herein above, wherein KNT (4) was replaced by XAGMSGASAGLYENKPRRPYIL (SEQ ID NO: 8, wherein X azido-acetyl lysine) to generate Compound No. 1252445. Similarly, a control compound 17-AE-9199 was generated according to Scheme 1b described herein above, wherein KNT (4) was replaced by [SO5P]LYEN[azido-acetyl-K]PRRPYIL peptide (SEQ ID NO: 4).

All the modified oligonucleotides in the table below are uniform MOE compounds, wherein every sugar moiety throughout the modified oligonucleotides is a 2′-MOE sugar moiety. The intemnucleoside linkages throughout the modified oligonucleotides in the table below are phosphodiester linkages, and are denoted by “o” in the table below. All cytosine residues are 5-methylcytosines. All the modified oligonucleotides are labeled with Cy3 at the 3′-end.

The attachment point of the peptide to the linker is underlined in the table below (Table 12). The compounds indicated in Table 12 are 10000 complementary to human MALAT RNA SEQ ID NO: 1 at start site 6925 to stop site 6944.

TABLE 12
Design of modified oligonucleotides

| | Se- | | | | | |
|---|---|---|---|---|---|---|
| Com- | quence | SEQ | 5′- | SEQ | 3′- | |
| pound | (5′ to | ID | conju- | ID | conju- | 5′- |
| No. | 3′) | NO | gate | NO | gate | linker |
| 1091165 | CCAGGCTGGT | 2 | SO5PLYEN | 4 | Cy3 | (o)-6-aminohexanol- |
| | TATGACTCAG | | KPRRPYIL | | (thioate) | 1- |
| | | | | | | carboxymethyl |
| | | | | | | [triazoloBCN1] |
| | | | | | | carbamate |
| 1162756 | CCAGGCTGGT | 2 | KPPPAGSSP | 5 | Cy3 | (o)-6-aminohexanol- |
| | TATGACTCAG | | GLYENKPR | | (thioate) | 1- |
| | | | RPYIL | | | carboxymethyl |
| | | | | | | [triazoloBCN1] |
| | | | | | | carbamate |
| 1252443 | CCAGGCTGGT | 2 | KPAPSGPSP | 6 | Cy3 | (o)-6-aminohexanol- |
| | TATGACTCAG | | GLYENKPR | | (thioate) | 1- |
| | | | RPYIL | | | carboxymethyl |
| | | | | | | [triazoloBCN1] |
| | | | | | | carbamate |
| 1252444 | CCAGGCTGGT | 2 | KAGSIKPPP | 7 | Cy3 | (o)-6-aminohexanol- |
| | TATGACTCAG | | AGSSPGLY | | (thioate) | 1- |
| | | | ENKPRRPYI | | | carboxymethyl |
| | | | L | | | [triazoloBCN1] |
| | | | | | | carbamate |
| 1252445 | CCAGGCTGGT | 2 | KAGMSGA | 8 | Cy3 | (o)-6-aminohexanol- |
| | TATGACTCAG | | SAGLYENK | | (thioate) | 1- |
| | | | PRRPYIL | | | carboxymethyl |
| | | | | | | [triazoloBCN1] |
| | | | | | | carbamate |

The modified oligonucleotides were tested in a series of experiments using the same culture conditions. HEK-SORT1 cells were seeded in 24-well plates (at ˜150,000 cells/well) and allowed to attach and equilibrate for at least 16 hours. The indicated Cy3-lableled compounds (U.S. Pat. Nos. 1,162,754, 1,091,165, 1,162,756, 17-AH-1493, 17-AE-9199 and 17-AH-1492 described herein above), were added to wells at the concentrations indicated in Table 13a below. In a separate experiment, the indicated Cy3-lableled compounds (17-AH-1492, 1016003, 1162756, 1252443, 1252444, and 1252445 described herein above), were added to wells at the concentrations indicated in table 13b below. Two plates were prepared for each treatment condition, one serving as 4° C. no internalization control that was kept on ice during incubation, while the second plate was incubated at 37° C. to allow for energy-dependent internalization. Following the incubation period all plates were placed on ice and washed 3 times with ice-cold PBS/3% BSA/2 mM EDTA then lifted with trypsin, diluted into wash buffer, and placed into flow cytometry tubes on ice. Cells were then analyzed by flow cytometry for mean fluorescence intensity using a BD FACS Calibur flow cytometer (BD Biosciences, San Jose, CA, USA). Internalized modified oligonucleotide was expressed as the difference between the corresponding 4° C. and 37° C. mean fluorescence intensities. In cases where data is not available, the value is marked N/A.

The data suggests that the extended peptide conjugate improves uptake of the neurotensin-conjugated modified oligonucleotides. Uptake of 1162756 containing the extended neurotensin peptide conjugate is comparable to 17-AH-1493.

TABLE 13a
Effect of extended peptide conjugate on modified oligonucleotide uptake

| Concen- | Mean Fluorescence Intensity (MFI) | | | | | |
|---|---|---|---|---|---|---|
| tration | 17-AH- | | 17-AE- | 17-AH- | | |
| (nM) | 1493 | 1162754 | 9199 | 1492 | 1091165 | 1162756 |
| 3000 | 1387 | 690 | 1383 | N/A | 138 | N/A |
| 1000 | 1229 | 293 | 1217 | 1264 | 97 | 1390 |
| 333 | 1126 | 171 | 1086 | 1034 | 73 | 812 |
| 111 | 928 | 71 | 825 | 724 | 60 | 246 |
| 37 | 362 | 54 | 378 | 390 | 53 | 119 |
| 12 | 126 | 43 | 174 | 167 | 45 | 67 |

TABLE 13b
Effect of extended peptide conjugate on modified oligonucleotide uptake

| Concen- | Mean Fluorescence Intensity (MFI) | | | | | |
|---|---|---|---|---|---|---|
| tration | 17-AH- | | | | | |
| (nM) | 1492 | 1016003 | 1162756 | 1252443 | 1252444 | 1252445 |
| 3000 | 1436 | 124 | 3380 | 3391 | 3388 | 2879 |
| 882 | 1182 | 63 | 2071 | 2427 | 2507 | 1954 |
| 260 | 869 | 47 | 937 | 1229 | 1534 | 868 |
| 76 | 593 | 44 | 309 | 413 | 609 | 306 |
| 22 | 266 | 37 | 122 | 130 | 208 | 85 |
| 7 | 84 | 35 | 51 | 53 | 90 | 55 |

In addition, Compound No. 1162756 was compared to a duplex of 1162756. The duplex was formed by complexing 1162756 to a complementary sequence (from 5′ to 3′) CTGAGTCATAACCAGCCTGG (SEQ ID NO: 78) wherein every nucleoside is a 2′-MOE sugar moiety, the intemucleoside linkages are all phosphodiester intemucleoside linkages. All cytosine residues are 5-methylcytosines.

HEK-SORT1 cells were seeded in 24-well plates (at ˜150,000 cells/well) and allowed to attach and equilibrate for at least 16 hours. The indicated Cy3-lableled compounds (1162756, 17-AH-1493, 1162756 duplex described herein above), were added to wells at the concentrations indicated in the table below. Two plates were prepared for each treatment condition, one serving as 4° C. no internalization control that was kept on ice during incubation, while the second plate was incubated at 37° C. to allow for energy-dependent internalization. Following the incubation period all plates were placed on ice and washed 3 times with ice-cold PBS/3% BSA/2 mM EDTA then lifted with trypsin, diluted into wash buffer, and placed into flow cytometry tubes on ice. Cells were then analyzed by flow cytometry for mean fluorescence intensity using a BD FACS Calibur flow cytometer (BD Biosciences, San Jose, CA, USA). Internalized modified oligonucleotide was expressed as the difference between the corresponding 4° C. and 37° C. mean fluorescence intensities. In cases where data is not available, the value is marked N/A.

TABLE 14
Effect of extended peptide conjugate
on modified oligonucleotide uptake

| | |
|---|---|
| Concentration | Mean Fluorescence Intensity (MFI) |

| (nM) | 17-AH-1492 | 1162756 | 1162756 duplex |
|---|---|---|---|
| 1000 | 1364 | 1563 | 278 |
| 333 | 1095 | 839 | 130 |
| 111 | 763 | 364 | 71 |
| 37 | 334 | 124 | 52 |
| 12 | 108 | 61 | 41 |

Synthesis, Binding, In Vitro and In Vivo Activity of 3-10-3 cET Gapmers with Extended Neurotensin Peptide Conjugates

Compound No. 786434 (38, described here in the table below) was conjugated to neurotensin peptide (4, KNT, XLYENKPRRPYIL (SEQ ID NO: 3), wherein X is azido-acetyl lysine) and the extended neurotensin peptide (25, XPPPAGSSPGLYENKPRRPYIL (SEQ ID NO: 5, wherein X is azido-acetyl lysine) as described below in Scheme 4 to generate Compound Nos 1166718 (35) and 1166356 (36) respectively.

The neurotensin 4 and extended neurotensin 25 were conjugated to Compound No. 38 (designated herein as Compound No. 786434) according to the synthetic method described in Scheme 4. The 5′-hexylamino modified Malat-1 modified oligonucleotide 38 was synthesized using previously described methods. The “ASO” in Compound 38 has the sequence (from 5′ to 3′): TCAGCATTCTAATAGCAGC (SEQ ID NO: 14), wherein nucleosides 4 to 19 having the sequence GCATTCTAATAGCAGC (SEQ ID NO: 15) are 100% complementary to mouse MALAT-1 RNA SEQ ID NO: 23 (GENBANK Accession No. NT_082868.4 (SEQ ID NO: 23) truncated from nucleotides 2689000 to 2699000) at position 6554 to 6569. Nucleosides 1 to 3 of Compound No. 38 having the sequence TCA, form a nuclease cleavable linker. The 5′-most nucleoside “T” is conjugated to either KNT or eNT peptides according to the method described briefly as follows. Compound 38 was treated with BCN—N-hydroxysuccinimide ester (NHS) at room temperature to yield BCN (bicyclo[6.1.0]nonyne) containing Compound 39. Compound 39 was then treated with neurotensin peptide 4, or extended neurotensin peptide 25 containing azido group at the N-terminus in DMSO-sodium tetraborate buffer (0.1 M, pH 8.5) at ambient temperature followed purification and desalting by HPLC. This yielded modified oligonucleotides 1166718 (described as Compound 35 in Scheme 4) and modified oligonucleotide 1166356 (described as Compound 36 in Scheme 4). The details of both conjugated compounds are specified in the table below.

The attachment point of the peptide to the linker is underlined in the table below. Compound No. 556089 in the table below has a sugar motif (from 5′ to 3′): kkkddddddddddkkk; wherein ‘d’ represents a 2′-β-D-deoxyribosyl sugar moiety, and ‘k’, which represents a cET sugar moiety. The internucleoside linkage motif for Compound No. 556089 is (from 5′ to 3′): sssssssssssssss; wherein ‘s’ represents a phosphorothioate internucleoside linkage. The sugar motif for the remaining modified oligonucleotides in the table below is (from 5′ to 3′): dddkkkddddddddddkkk; wherein ‘d’ represents a 2′-p-D-deoxyribosyl sugar moiety, and ‘k’, which represents a cET sugar moiety. The internucleoside linkage motif for the remaining modified oligonucleotides in the table below is (from 5′ to 3′): ooosssssssssssssss; wherein each ‘o’ represents a phosphodiester internucleoside linkage and each ‘s’ represents a phosphorothioate internucleoside linkage. All cytosine residues are 5-methylcytosines. Compound No. 556089 is an unconjugated parent compound that has 100% complementarity to mouse MALAT-1 RNA SEQ ID NO: 23. The remaining modified oligonucleotides in the table below have the sequence (from 5′ to 3′): TCAGCATTCTAATAGCAGC (SEQ ID NO: 14), wherein nucleosides 4 to 19 having the sequence GCATTCTAATAGCAGC (SEQ ID NO: 15) are 100% complementary to mouse MALAT-1 RNA SEQ ID NO: 23 (GENBANK Accession No. NT_082868.4 (SEQ ID NO: 23) truncated from nucleotides 2689000 to 2699000) at position 6554 to 6569. Nucleosides 1 to 3 of the modified oligonucleotides having the sequence TCA, form a nuclease cleavable linker.

TABLE 15
Design of modified oligonucleotides

| | Se- | | | | | |
|---|---|---|---|---|---|---|
| Com- | quence | SEQ | 5′- | SEQ | 3′- | |
| pound | (5′ to | ID | conju- | ID | conju- | 5′- |
| No. | 3′) | NO | gate | NO | gate | linker |
| 556089 | GCATT | 15 | none | N/A | none | none |
| | CTAAT | | | | | |
| | AGCAG | | | | | |
| | C | | | | | |
| 786434 | TCAGC | 14 | (o)-6- | N/A | none | none |
| | ATTCT | | amino | | | |
| | AATAG | | hexanol | | | |
| | CAGC | | | | | |
| 1166718 | TCAGC | 14 | dKLY | 3 | none | (o)-6- |
| | ATTCT | | ENKP | | | aminohexanol- |
| | AATAG | | RRPY | | | 1- |
| | CAGC | | IL | | | carboxymethyl |
| | | | dKLY | | | [triazoloBCN1] |
| | | | | | | carbamate |
| 1166356 | TCAGC | 14 | KPPP | 5 | none | (o)-6- |
| | ATTCT | | AGSS | | | aminohexanol- |
| | AATAG | | PGLY | | | 1- |
| | CAGC | | ENKP | | | carboxymethyl |
| | | | RRPY | | | [triazoloBCN1] |
| | | | IL | | | carbamate |

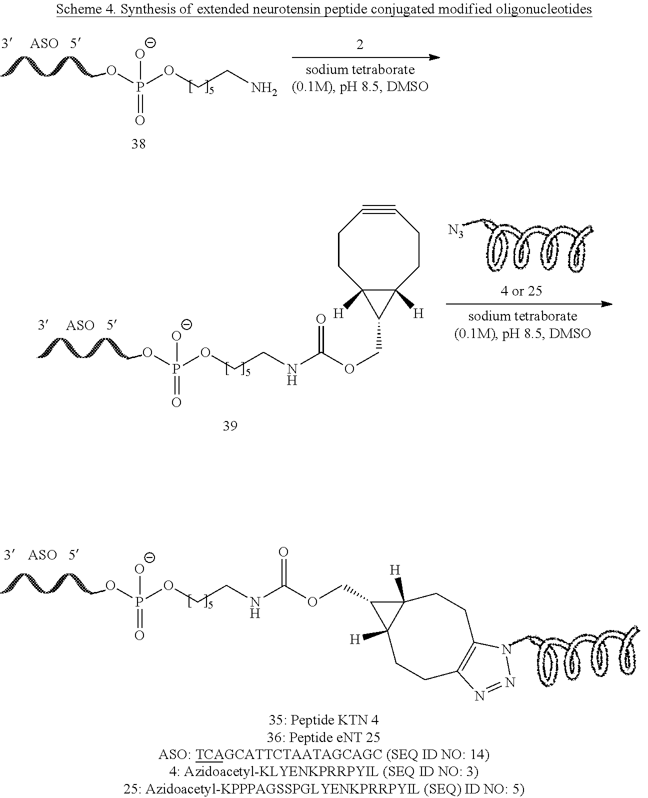

The conjugates 1166718 and 1166356 were evaluated in a ligand-receptor binding assay. This competition binding assay measures the change in fluorescence polarization upon displacement of an Alexa Fluor 647-labeled tracer ligand from the corresponding receptor. The assay was set up in 96-well costar plates (black flat-bottomed non-binding) purchased from Corning, NY, USA in phosphate buffered saline (1× PBS). Competitive binding was evaluated at an ALEXA647-labeled neurotensin concentration of 2 nM, a sortilin concentration of 625 nM, and oligonucleotide conjugate concentration ranging from 100 pM up to 400 μM. Readings were taken using the Tecan (Baldwin Park, CA, USA) InfiniteM1000 Pro instrument ((lₑₓ=635 nm, lₑₘ=675 nm). Using polarized excitation and emission filters, the instrument measures fluorescence perpendicular to the excitation plane (the ‘P-channel’) and fluorescence that is parallel to the excitation plane (the ‘S-channel’), and then it calculates FP in millipolarization units (mP) as follows: mP=[(S−P*G)/(S+P*G)]*1000. The ‘G-factor’ is measured by the instrument as a correction for any bias toward the P channel. Polarization values of each Alexa 647-labeled modified oligonucleotide in 1× PBS at 2 nM concentration were subtracted from each measurement. Ki values were calculated with GraphPad Prism 5 software (GraphPad Software, La Jolla, CA, USA) using non-linear regression for curve fit assuming one binding site. As shown in the table below, the conjugate of extended neurotensin (1166356) bound three times tighter than that of parent neurotensin (1166718).

TABLE 16
Competitive displacement of Alexa Fluor-labeled
KNT from sortilin by modified oligonucleotides

| | |
|---|---|
| Concentration | ΔFP |

| (M) | 1166718 | 1166356 |
|---|---|---|
| 3.97E−04 | 14 | 13 |
| 4.41E−05 | 15 | 0 |
| 1.47E−05 | 32 | 9 |
| 4.90E−06 | 56 | 57 |
| 1.63E−06 | 86 | 89 |
| 5.44E−07 | 83 | 80 |
| 1.81E−07 | 95 | 97 |
| 6.05E−08 | 86 | 97 |
| 2.02E−08 | 89 | 96 |
| 6.72E−09 | 91 | 102 |
| 2.24E−09 | 90 | 106 |

To determine potency of the modified oligonucleotides 1166718 and 1166356 in vitro, HEK-SORT1 cells were seeded in 96-well plates (at ˜150,000 cells/well) and transfected with the compounds at the concentrations shown in the tables below. The levels of MALAT in the cells after a 24 hr incubation with the modified oligonucleotides was measured using quantitative RTPCR.

TABLE 17
Reduction of MALAT-1 RNA by modified oligonucleotides

| Concentration | % UTC | | | |
|---|---|---|---|---|
| (nM) | 556089 | 1146627 | 1166356 | 1166718 |
| 20000 | 19 | 23 | 7 | 9 |
| 6667 | 21 | 27 | 11 | 12 |
| 2222 | 29 | 30 | 16 | 16 |
| 741 | 38 | 49 | 25 | 23 |
| 247 | 59 | 69 | 45 | 37 |
| 82 | 66 | 82 | 53 | 52 |
| 27 | 83 | 99 | 70 | 53 |
| 9. | 86 | 97 | 71 | 61 |
| 3. | 92 | 106 | 94 | 88 |
| 1.0 | 94 | 102 | 99 | 93 |
| 0.34 | 115 | 98 | 111 | 101 |
| 0.11 | 100 | 96 | 91 | 99 |

To determine potency of Compound No. 1166356 in vivo, the modified oligonucleotides (Compound No. 1166356 and unconjugated parent Compound No. 556089) were tested in C57B16/J female mice. The mice were divided into groups of 4 mice each. Each mouse received a single ICV bolus of compound at 0.25, 1, 3, 7.5, 10, 30, or 100 g of modified oligonucleotide and sacrificed two weeks later. A group of 4 mice received PBS as a negative control.

### RNA Analysis

After two weeks, mice were sacrificed, and RNA was extracted from cortical brain tissue and spinal cords for real-time PCR analysis of measurement of RNA expression of MALAT using primer probe set mMALAT1 #2 (forward sequence TGGGTTAGAGAAGGCGTGTACTG, designated herein as SEQ ID NO: 24; reverse sequence TCAGCGGCAACTGGGAAA, designated herein as SEQ ID NO: 25; probe sequence CGTTGGCACGACACCTTCAGGGACT, designated herein as SEQ ID NO: 26). Results are presented as percent change of RNA, relative to PBS control, normalized to mouse GAPDH, measured by primer-probe set mGapdh_LTS00102 (forward sequence GGCAAATTCAACGGCACAGT, designated herein as SEQ ID NO: 27; reverse sequence GGGTCTCGCTCCTGGAAGAT, designated herein as SEQ ID NO: 28; probe sequence AAGGCCGAGAATGGGAAGCTTGTCATC, designated herein as SEQ ID NO: 29).

As shown in Table 18, treatment with peptide-conjugated modified oligonucleotide resulted enhanced knockdown of MALAT1 in comparison to the parent compound in cortex.

TABLE 18
Dose-dependent percent reduction of mouse MALAT RNA in vivo

| | | CORTEX | | SPINAL CORD | |
|---|---|---|---|---|---|
| Compound | Dose | MALAT RNA | ED50 | MALAT RNA | ED50 |
| ID | (μg) | (% control) | (μg) | (% control) | (μg) |
| 556089 | 100 | 16 | 35.4 | 3 | 2.1 |
| | 30 | 75 | | 5 | |
| | 10 | 61 | | 14 | |
| | 7.5 | 75 | | 28 | |
| | 3 | 100 | | 38 | |
| | 1 | 99 | | 70 | |
| | 0.25 | 92 | | 87 | |
| 1166356 | 100 | 57 | 113.6 | 2 | 2.1 |
| | 30 | 81 | | 4 | |
| | 10 | 107 | | 7 | |
| | 7.5 | 114 | | 19 | |
| | 3 | 116 | | 41 | |
| | 1 | 125 | | 72 | |
| | 0.25 | 109 | | 92 | |

### Example 8: Synthesis and In Vivo Activity of Sortilin Propeptide Conjugated 3-10-3 cET Gapmer Oligonucleotides

The 5′-hexylamino modified oligonucleotide 38 (Compound No. 786434, described herein above) that targets MALAT-1 was treated with N-succinimidyl 3-maleimidopropionate 28 at room temperature to yield 5′-(3-maleimidyl)propionyl-hexylamino modified oligonucleotide 40. Compound 40 was then treated with sortilin propeptide 31 (CQDRLDAPPPPAAPLPRWSGPIGVSWGLRAAAAGGAFPRGGRWRR, SEQ ID NO: 76) at ambient temperature for 3 hours, followed by purification and desalting by HPLC to yield propeptide conjugated Malat-1 modified oligonucleotide 37 (designated herein as Compound No. 1272118).

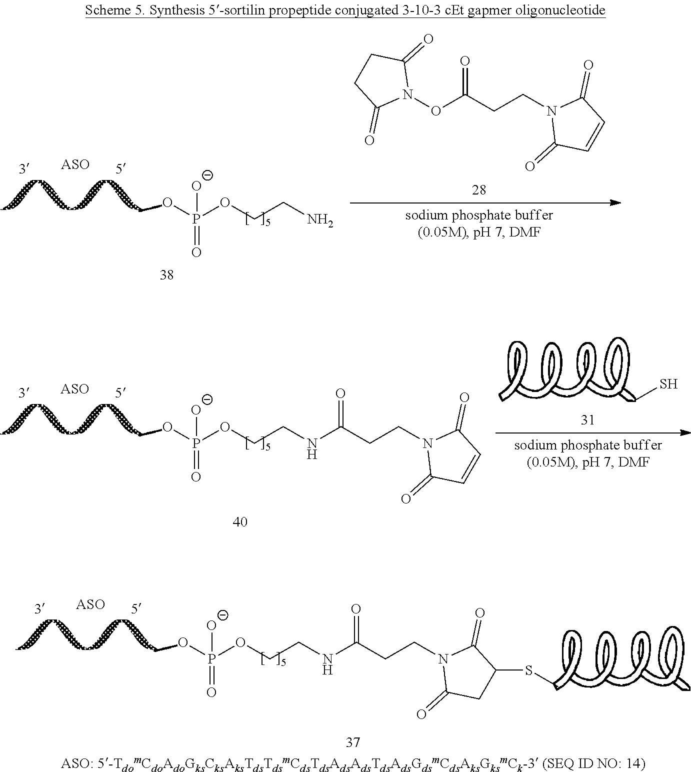

The details of Compound No. 1272118 are detailed in the table below. The attachment point of the peptide to the conjugate linker is underlined in the table below. The sugar motif for the modified oligonucleotide in the table below is (from 5′ to 3′): dddkkkddddddddddkkk; wherein ‘d’ represents a 2′-β-D-deoxyribosyl sugar moiety, and ‘k’, which represents a cET sugar moiety. The internucleoside linkage motif for the modified oligonucleotide in the table below is (from 5′ to 3′): ooosssssssssssssss; wherein each ‘o’ represents a phosphodiester internucleoside linkage and each ‘s’ represents a phosphorothioate internucleoside linkage. All cytosine residues are 5-methylcytosines.

TABLE 19
Design of modified oligonucleotides

| | Se- | | | | | |
|---|---|---|---|---|---|---|
| Com- | quence | SEQ | 5′- | SEQ | 3′- | |
| pound | (5′ to | ID | conju- | ID | conju- | 5′- |
| No. | 3′) | NO | gate | NO | gate | linker |
| 1272118 | TCAGC | 14 | CQDRL | 76 | none | (o)-6- |
| | ATTCT | | DAPPP | | | aminohexanol- |
| | AATAG | | PAAPL | | | maleimideC3 |
| | CAGC | | PRWSG | | | |
| | | | PIGVS | | | |
| | | | WGLRA | | | |
| | | | AAAGG | | | |
| | | | AFPRG | | | |
| | | | GRWRR | | | |

To evaluate whether neurotensin conjugation improved potency of modified oligonucleotides in the central nervous system, two in vivo activity studies were carried out. Compound Nos. 1166356 (described herein above), 556089 (described herein above) and 1272118 (details described in table below) were administered by intracerebroventricular bolus injection into the lateral ventricle of mice (4 mice were treated per group). Mice were sacrificed 14 days post treatment and Malat-1 RNA expression was analyzed in the spinal cord, cortex, striatum and cerebellum using quantitative RT-PCR. Compound Nos. 1166356 and 1272118 showed improved Malat-1 RNA reduction in the spinal cord relative to unconjugated Compound No. 556089. Results are presented as percent mouse MALAT-1 RNA relative to PBS control, normalized to mouse GAPDH.

TABLE 20
Reduction of Mouse MALAT-1 in mice

| Compound | | | Concentration (μg) | | | | | |
|---|---|---|---|---|---|---|---|---|
| No. | Tissue | | UTC | 0.25 | 1 | 3.5 | 10 | 30 |
| 556089 | Spinal Cord | MALAT-1 RNA | 100 | 92 | 78 | 28 | 13 | 5 |
| | | (% UTC) | | | | | | |
| | | ED₅₀ μg | 2.55 | | | | | |
| | Cortex | MALAT-1 RNA | 100 | 102 | 102 | 110 | 97 | 30 |
| | | (% UTC) | | | | | | |
| | | ED50 μg | 23.48 | | | | | |
| | Striatum | MALAT-1 RNA | 100 | 100 | 99 | 63 | 36 | 13 |
| | | (% UTC) | | | | | | |
| | | ED50 μg | 18.34 | | | | | |
| | Cerebellum | MALAT-1 RNA | 100 | 104 | 104 | 83 | 57 | 35 |
| | | (% UTC) | | | | | | |
| | | ED50 μg | 17.11 | | | | | |
| 1166356 | Spinal Cord | MALAT-1 RNA | 100 | 78 | 53 | 23 | 11 | 5 |
| | | (% UTC) | | | | | | |
| | | ED50 μg | 1.03 | | | | | |
| | Cortex | MALAT-1 RNA | 100 | 96 | 94 | 74 | 80 | 31 |
| | | (% UTC) | | | | | | |
| | | ED50 μg | 19.43 | | | | | |
| | Striatum | MALAT-1 RNA | 100 | 90 | 92 | 68 | 42 | 22 |
| | | (% UTC) | | | | | | |
| | | ED50 μg | 17.39 | | | | | |
| | Cerebellum | MALAT-1 RNA | 100 | 97 | 92 | 75 | 61 | 37 |
| | | (% UTC) | | | | | | |
| | | ED50 μg | 18.68 | | | | | |
| 1272118 | Spinal Cord | MALAT-1 RNA | 100 | 90 | 54 | 30 | 10 | 5 |
| | | (% UTC) | | | | | | |
| | | ED50 μg | 1.35 | | | | | |
| | Cortex | MALAT-1 RNA | 100 | 94 | 74 | 72 | 63 | 51 |
| | | (% UTC) | | | | | | |
| | | ED50 μg | 24.74 | | | | | |
| | Striatum | MALAT-1 RNA | 100 | 84 | 85 | 79 | 64 | 39 |
| | | (% UTC) | | | | | | |
| | | ED50 μg | 62.89 | | | | | |
| | Cerebellum | MALAT-1 RNA | 100 | 92 | 94 | 60 | 52 | 37 |
| | | (% UTC) | | | | | | |

| | |
|---|---|
| ED50 μg | 12.9 |

### Example 9: Synthesis and In Vivo Activity of Neurotensin Conjugated Splice Modulating Phosphoramidite Morpholino Oligomer

Compound 41 (GeneTools LLC Catalog 699822, designated herein as Compound No. 709034) has the sequence (from 5′ to 3′) ATTCACTTTCATAATGCTGG (SEQ ID NO: 79), wherein every nucleobase is a morpholino and the internucleoside linkages are phosphorodiamidate linkages throughout the compound. Compound No. 709034 is 100% complementary to human SMN2 sequence SEQ ID NO: 80 (GENBANK Accession No. NT_006713.14 truncated from nucleotides 19939708 to 19967777) from positions 27062 to 27081. Compound 41 was reacted with BCN—N-hydroxysuccinimide ester (2) in DMSO to generate a BCN functionalized morpholino oligomer 43. Compound 43 was then reacted with neurotensin peptide 4 (KNT, XLYENKPRRPYIL (SEQ ID NO: 3), wherein X is azido-acetyl lysine, described herein above) containing azido group at N-terminus in DMSO-sodium tetraborate buffer (0.1M, pH 8.5) at room temperature to yield Compound 42 (designated herein as Compound No. 1297806). Compound No. 1297806 was purified by HPLC.

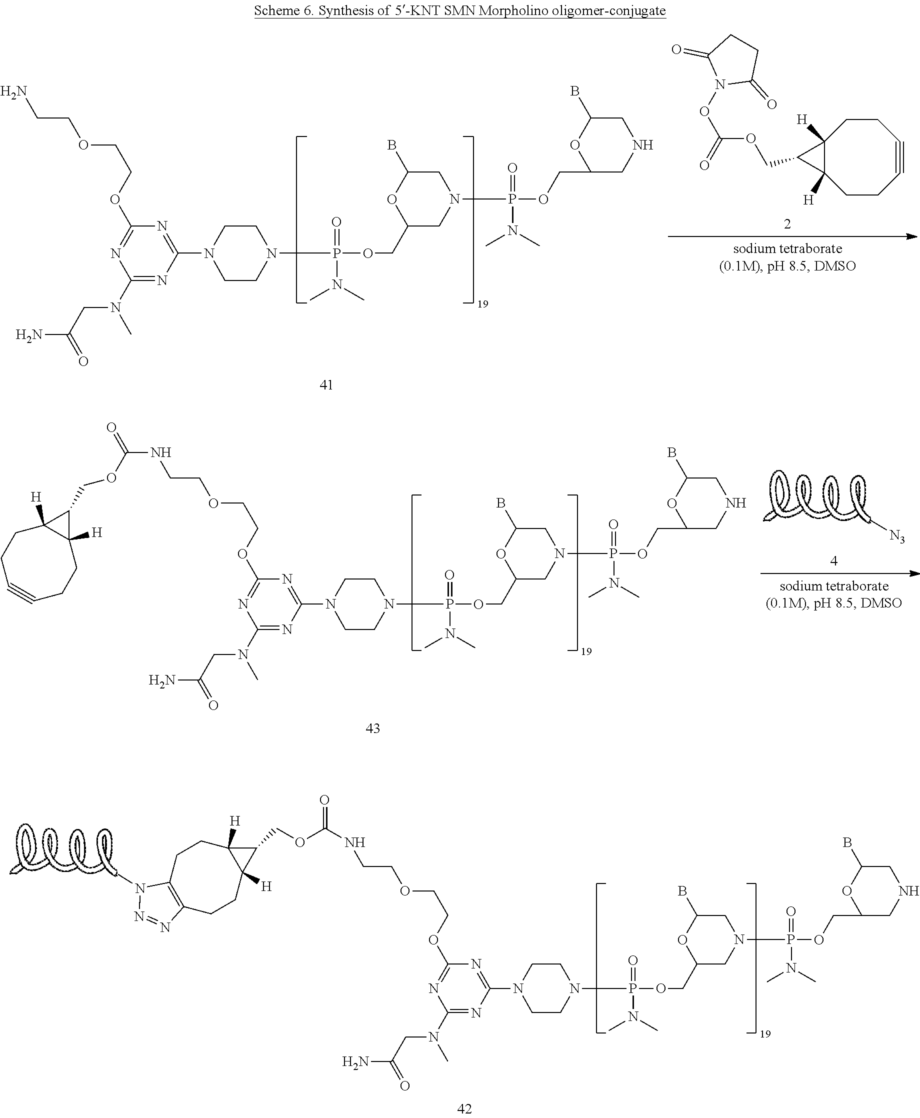

To evaluate the potency of Compound No. 1297806, transgenic SMA mice were given a single ICV bolus injection of modified oligonucleotides 709034 and 1297806 at 10 μg, 30 μg and 100 μg. The transgenic mice were divided into groups of 4 mice each. One group of 4 mice were treated with PBS as a control. Mice were sacrificed 14 days post treatment. Cortical and striatal tissues were extracted and analyzed for SMN2 splicing correction (exon 7 inclusion) using quantitative RT-PCR.

TABLE 21
Effect of modified oligonucleotides on human SMN2 RNA splicing in transgenic mice

| Compound | Dose | STRIATUM | | CEREBELLUM | | CORTEX | |
|---|---|---|---|---|---|---|---|
| No. | (μg) | exon 7⁺ | exon 7⁻ | exon 7⁺ | exon 7⁻ | exon 7⁺ | exon 7⁻ |
| PBS | 0 | 1 | 1 | 1 | 1 | 1 | 1 |
| 709034 | 10 | 1.2 | 0.8 | 1.5 | 0.8 | 1.8 | 0.8 |
| | 30 | 1.5 | 0.6 | 2.1 | 0.6 | 2.9 | 0.6 |
| | 100 | 2.1 | 0.5 | 3.1 | 0.4 | 4.2 | 0.3 |
| 1297806 | 10 | 1.3 | 0.7 | 1.5 | 0.7 | 2.3 | 0.7 |
| | 30 | 1.8 | 0.6 | 2 | 0.6 | 3.9 | 0.4 |
| | 100 | 2.6 | 0.4 | 2.7 | 0.5 | 4.9 | 0.2 |

### Example 10: Effect of Maleimide Linker on Neurotensin Peptide Conjugated Modified Oligonucleotide Uptake into HEK-SORT1 Cells

Modified oligonucleotide with a maleimide linker were designed per description in the table below. All the modified oligonucleotide in the table below are uniform MOE compounds, wherein every sugar moiety throughout the modified oligonucleotides is a 2′-MOE sugar moiety. The internucleoside linkages throughout the modified oligonucleotide in the table below are phosphodiester linkages, and are denoted by “o” in the table below. All cytosine residues are 5-methylcytosines. All compounds are labeled with Cy3 at the 3′-end.

The conjugated modified oligonucleotide was synthesized according to the method shown in Scheme 3 herein above, wherein the Compounds 30 or 31 in Scheme 3 were replaced with the peptide sequence CPPPAGSSPGLYENKPRRPYIL (SEQ ID NO: 13) to generate Compound No. 1215812. The attachment point of the peptide to the linker is underlined in the table below (Table 22). 1215812 is 100% complementary to human MALAT RNA SEQ ID NO: 1 at position 6925 to 6944.

TABLE 22
Design of modified oligonucleotides

| | Se- | | | | | |
|---|---|---|---|---|---|---|
| Com- | quence | SEQ | 5′- | SEQ | 3′- | |
| pound | (5′ to | ID | conju- | ID | conju- | 5′- |
| No. | 3′) | NO | gate | NO | gate | linker |
| 1215812 | CCAGG | 2 | CPPPA | 13 | Cy3 | (o)-6- |
| | CTGGT | | GSSPG | | (thioate) | aminohexanol- |
| | TATGA | | LYENK | | | maleimideC3 |
| | CTCAG | | PRRPY | | | |
| | | | IL | | | |

HEK-SORT1 cells were seeded in 24-well plates (at ˜150,000 cells/well) and allowed to attach and equilibrate for at least 16 hours. The indicated Cy3-lableled compounds (17-AH-1493, 1162754, 1162756 and 1215812 described herein above), were added to wells at the concentrations indicated in Table 23 below. Two plates were prepared for each treatment condition, one serving as 4° C. no internalization control that was kept on ice during incubation, while the second plate was incubated at 37° C. to allow for energy-dependent internalization. Following the incubation period all plates were placed on ice and washed 3 times with ice-cold PBS/3% BSA/2 mM EDTA then lifted with trypsin, diluted into wash buffer, and placed into flow cytometry tubes on ice. Cells were then analyzed by flow cytometry for mean fluorescence intensity using a BD FACS Calibur flow cytometer (BD Biosciences, San Jose, CA, USA). Internalized modified oligonucleotide was expressed as the difference between the corresponding 4° C. and 37° C. mean fluorescence intensities.

The data indicates that both the click and the maleimide linkers showed similar uptake of the neurotensin-conjugated modified oligonucleotides.

TABLE 23
Effect of maleimide linker on Neurotensin
peptide conjugated oligonucleotide uptake

| Concentration | Mean Fluorescence Intensity (MFI) | | | |
|---|---|---|---|---|
| (nM) | 17-AH-1493 | 1162754 | 1162756 | 1215812 |
| 3000 | 1389 | 1299 | 4217 | 3145 |
| 882 | 1209 | 381 | 2103 | 1806 |
| 260 | 710 | 145 | 817 | 675 |
| 76 | 266 | 73 | 259 | 205 |
| 22 | 63 | 50 | 102 | 69 |
| 7 | 46 | 43 | 53 | 47 |

### Example 11: Design, Synthesis, Tolerability, and In Vivo Activity of Sortilin Propeptide Conjugated RNAi Compounds in Wild-Type Mice

### Design of Sortilin Propeptide Conjugated RNAi Compounds

RNAi compounds comprising antisense RNAi oligonucleotides complementary to a mouse HPRT1 nucleic acid and sense RNAi oligonucleotides complementary to the antisense RNAi oligonucleotides were designed as follows.

The RNAi compounds in the table below consist of an antisense RNAi oligonucleotide and a sense RNAi oligonucleotide. The antisense RNAi oligonucleotide 1586322 has the sequence UAAAAUCUACAGUCAUAGGAAU (SEQ ID NO: 30), is 23 nucleosides in length, has a 5′-vinyl phosphonate, has a sugar motif (from 5′ to 3′) of: efyyyfyyyyyyyfyfyyyyyyy, wherein each “y” represents a 2′-O-methylribosyl sugar, each “f” represents a 2′-fluororibosyl sugar, and each “e” represents a 2′-MOE sugar, and has an internucleoside linkage motif (from 5′ to 3′) of: ssooooooooooooooooooss, wherein each “o” represents a phosphodiester internucleoside linkage, and each “s” represents a phosphorothioate internucleoside linkage. Each sense RNAi oligonucleotide in the table below is 21 nucleosides in length; has a sugar motif (from 5′ to 3′) of: yyyyyyfyfffyyyyyyyyyy, wherein each “y” represents a 2′-O-methylribosyl sugar, and each “f” represents a 2′-fluororibosyl sugar; and has an internucleoside linkage motif (from 5′ to 3′) of: ssooooooooooooooooss, wherein each “o” represents a phosphodiester internucleoside linkage, and each “s” represents a phosphorothioate internucleoside linkage.

The antisense RNAi oligonucleotide 1586322 is 100% complementary to the target nucleic acid (HPRT1), GenBank Accession No. NM_000194.2 (SEQ ID NO: 31) from position 444 to 465. Each sense RNAi oligonucleotide is complementary to the first 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides).

The N-terminal cysteine of the sortilin propeptide is conjugated to the 3′-end of 1615554, the sense RNAi oligonucleotide of Compound No. 1616034, through a (o)-6-aminohexanol-maleimideC3 linker (Glen Research, catalog number 20-2958-xx) The attachment point of the peptide to the conjugate linker is underlined in the table below.

TABLE 24
Design of siRNA sense oligonucleotides

| | | | 3′- | | 3′- |
|---|---|---|---|---|---|
| | Sense | | conjugate | | linker |
| | Se- | | on | | on |
| | quence | SEQ | sense | SEQ | sense |
| Sense | (5′ to | ID | oligo- | ID | oligo- |
| ID | 3′) | NO | nucleotide | NO | nucleotide |
| 1586323 | UCCUAU | 35 | none | N/A | none |
| | GACUGU | | | | |
| | AGAUU | | | | |
| | UUAA | | | | |
| 1615554 | UCCUAU | 35 | CQDRL | 76 | (o)-6- |
| | GACUGU | | DAPPP | | aminohexanol- |
| | AGAUU | | PAAPL | | maleimideC3 |
| | UUAA | | PRWSG | | |
| | | | PIGVS | | |
| | | | WGLRA | | |
| | | | AAAGG | | |
| | | | AFPRG | | |
| | | | GRWRR | | |

TABLE 25
Design of RNAi compounds

| Compound No. | Antisense ID | Sense ID |
|---|---|---|
| 1616033 | 1586322 | 1586323 |
| 1616034 | 1586322 | 1615554 |

### Synthesis of Sortilin Propeptide Conjugated RNAi Compounds

To synthesize Compound No. 1615554, the 3′-hexylamino modified oligonucleotide having the sequence UCCUAUGACUGUAGAUUUUAA (SEQ ID NO: 35), the sugar motif (from 5′ to 3′): yyyyyyfyfffyyyyyyyyyy, wherein each “y” represents a 2′-O-methylribosyl sugar, and each “f” represents a 2′-fluororibosyl sugar; and the internucleoside linkage motif (from 5′ to 3′): ssooooooooooooooooss, wherein each “o” represents a phosphodiester internucleoside linkage, and each “s” represents a phosphorothioate internucleoside linkage, (Compound No. 1590184, 44) at 1 μmol was dissolved in 0.1 M sodium phosphate buffer (0.6 mL/μmol, pH 7.2). To this, N-succinimidyl 3-maleimidopropionate 28 (5 mol) in 0.1 mL/μmol DMSO was added. The reaction mixture was stirred at room temperature for 2 h. Water (5 mL/μmol) was added and the resulting solution was purified by HPLC on a strong anion exchange column (GE Healthcare Life Sciences SOURCE 30Q) using a linear gradient of buffer A (100 mM NH₄OAc in water containing 30%) to buffer B (11.5M NaBr), and desalted by HPLC on a reverse phase column. To a solution of 1.0 μmol 5′-(3-maleimidyl)propionyl-hexylamino modified oligonucleotide (45) dissolved in degassed 0.1M sodium phosphate buffer (1.0 mL/μmol, pH 7.2), a solution of the propeptide (1.3-2 μmol) containing a free cysteine in degassed DMF (0.25 mL/μmol) was added. Peptides were functionalized with a cysteine moiety at the C-terminus (31) and conjugated to the maleimide-functionalized modified oligonucleotide (45) according to Scheme 7 to yield 46 (designated herein as Compound No. 1615554, with the conjugated peptide CQDRLDAPPPPAAPLPRWSGPIGVSWGLRAAAAGGAFPRGGRWRR, SEQ ID NO: 76). The reaction mixture was stirred at room temperature for 2-3 h. Progress of the reaction was monitored by LC-MS analysis. After the completion of reaction as assessed by LC-MS analysis, reaction was diluted with water 5 mL/μmol) and purified by HPLC on a strong anion exchange column (GE Healthcare Life Sciences SOURCE 30Q) using a linear gradient of buffer A (100 mM NH₄OAc in water containing 30%) to buffer B (11.5M NaBr), and desalted by HPLC on a reverse phase column.

Compound 46 in Scheme 7 is 1615554, described herein above. Each sense modified oligonucleotide was separately hybridized with the antisense RNAi oligonucleotide 1586322 to form the duplex Compounds No. 1616033 and No. 1616034.

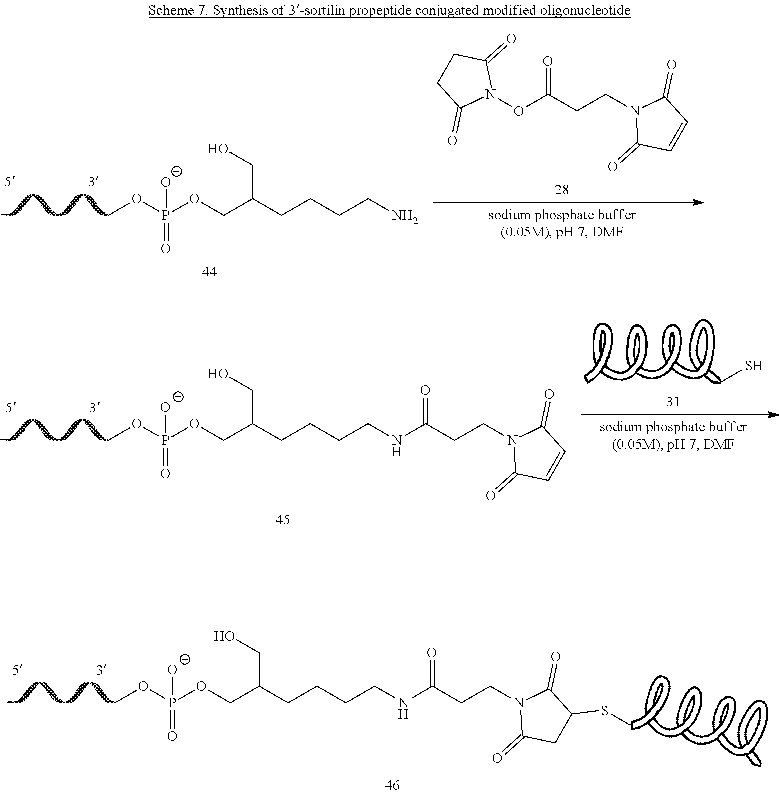

### In Vivo Study Design

In vivo studies were carried out to evaluate whether neurotensin conjugation improved potency of RNAi compounds in the central nervous system. The RNAi compounds described above were tested in C57B16/J female mice. The mice were divided into groups of 2 mice each. Each mouse received a single ICV bolus of compound at 0.0007, 0.007, 0.07, 0.7, 2.4, and/or 4.7 μmol of RNAi compound and sacrificed two weeks later. A group of 4 mice received PBS as a negative control.

### Tolerability of Sortilin Propeptide Conjugated RNAi Compounds

At 3 hours post-injection, mice were evaluated according to seven different criteria. The criteria are (1) the mouse was bright, alert, and responsive; (2) the mouse was standing or hunched without stimuli; (3) the mouse showed any movement without stimuli; (4) the mouse demonstrated forward movement after it was lifted; (5) the mouse demonstrated any movement after it was lifted; (6) the mouse responded to tail pinching; (7) regular breathing. For each of the 7 criteria, a mouse was given a subscore of 0 if it met the criteria and 1 if it did not (the functional observational battery score or FOB). After all 7 criteria were evaluated, the scores were summed for each mouse. The results are presented in the table below.

RNAi conjugated to sortilin propeptide (Compound No. 1616034) has similar tolerability in mouse as the parent RNAi (Compound No. 1616033).

TABLE 25
Tolerability of RNAi compounds

| Compound No. | Dose (μmol) | 3 hour FOB |
|---|---|---|
| PBS | N/A | 0, 0, 0, 0 |
| 1616033 | 0.0007 | 0, 0 |
| | 0.007 | 0, 0 |
| | 0.07 | 0, 0 |
| | 0.7 | 0, 0 |
| | 2.4 | N/A |
| | 4.7 | 1, 1 |
| 1616034 | 0.0007 | 0, 0 |
| | 0.007 | 1, 1 |
| | 0.07 | 0, 1 |
| | 0.7 | 1, 1 |
| | 2.4 | 1, 1 |
| | 4.7 | N/A |

### RNA Analysis of Sortilin Propeptide Conjugated RNAi Compounds

After two weeks, mice were sacrificed, and RNA was extracted from spinal cord, cortex, striatum and cerebellum for real-time PCR analysis of measurement of RNA expression of HPRT1 using primer-probe set RTS43125 (forward sequence CTCCTCAGACCGCTTTTTGC, designated herein as SEQ ID NO: 17; reverse sequence TAACCTGGTTCATCATCGCTAATC, designated herein as SEQ ID NO: 18; probe sequence CCGTCATGCCGACCCGCAGT, designated herein as SEQ ID NO: 19). Results are presented as percent mouse HPRT1 RNA relative to the amount in PBS treated mice (% ctrl), normalized to mouse cyclophilin A, measured by primer-probe set m_cyclo24 (forward sequence TCGCCGCTTGCTGCA, designated herein as SEQ ID NO: 20; reverse sequence ATCGGCCGTGATGTCGA, designated herein as SEQ ID NO: 21; probe sequence CCATGGTCAACCCCACCGTGTTC, designated herein as SEQ ID NO: 22).

As shown in the table below, treatment with peptide-conjugated RNAi (Compound No. 1616034) resulted in enhanced knockdown of HPRT1 in comparison to the parent compound (Compound No. 1616033) in cortex, thoracic cord, and striatum.

TABLE 26
Reduction of mouse HPRT1 RNA

| | | Spinal Cord | | Cortex | | Striatum | | Cerebellum | |
|---|---|---|---|---|---|---|---|---|---|
| | | HPRT1 | | HPRT1 | | HPRT1 | | HPRT1 | |
| Compound | Dose | RNA | ED₅₀ | RNA | ED₅₀ | RNA | ED₅₀ | RNA | ED₅₀ |
| No. | (μmol) | (% ctrl) | (μmol) | (% ctrl) | (μmol) | (% ctrl) | (μmol) | (% ctrl) | (μmol) |
| PBS | N/A | 100 | N/A | 100 | N/A | 100 | N/A | 100 | N/A |
| 1616033 | 0.0007 | 97 | 0.5 | 110 | 0.7 | 96 | 0.7 | 107 | 0.8 |
| | 0.007 | 104 | | 110 | | 94 | | 104 | |
| | 0.07 | 100 | | 98 | | 88 | | 86 | |
| | 0.7 | 35 | | 47 | | 51 | | 51 | |
| | 2.4 | N/A | | N/A | | N/A | | N/A | |
| | 4.7 | 14 | | 9 | | 10 | | 23 | |
| 1616034 | 0.0007 | 109 | 0.2 | 112 | 0.4 | 101 | 0.4 | 98 | 1.4 |
| | 0.007 | 98 | | 89 | | 87 | | 87 | |
| | 0.07 | 72 | | 79 | | 69 | | 76 | |
| | 0.7 | 29 | | 44 | | 48 | | 56 | |
| | 2.4 | 15 | | 10 | | 21 | | 45 | |
| | 4.7 | N/A | | N/A | | N/A | | N/A | |

1. An oligomeric compound comprising an oligonucleotide and a conjugate group comprising a conjugate moiety, and a conjugate linker that links the conjugate moiety to the oligonucleotide,
wherein the conjugate moiety comprises a cell-targeting peptide comprising an amino acid sequence that is at least 75%, at least 80%, at least 85%, at least 90%, or at least 95% identical to an amino acid sequence selected from XLYENKPRRPYIL (SEQ ID NO: 3) LYENKPRRPYIL (SEQ ID NO: 32), ELYENKPRRPYIL (SEQ ID NO: 72), XLYENKPKRPYIL (SEQ ID NO: 73), XLYENKPRKPYIL (SEQ ID NO: 74), and XLYENKPKKPYIL (SEQ ID NO: 75), wherein X is lysine or 2-azido-acetyl lysine.

2.-7. (canceled)

8. The oligomeric compound of claim 1, wherein the conjugate moiety comprises a peptide extender that extends the cell-targeting peptide.

9. (canceled)

10. The oligomeric compound of claim 8, wherein the amino acid sequence of the peptide extender and cell-targeting peptide together is at least 75%, at least 80%, at least 85%, at least 90%, at least 95%, or 100% identical to an amino acid sequence selected from XPPPAGSSPGLYENKPRRPYIL (SEQ ID NO: 5), XPAPSGPSPGLYENKPRRPYIL (SEQ ID NO: 6), XAGSIKPPPAGSSPGLYENKPRRPYIL (SEQ ID NO: 7), and XAGMSGASAGLYENKPRRPYIL (SEQ ID NO: 8), wherein X is lysine or azido-acetyl lysine.

11.-21. (canceled)

22. The oligomeric compound of claim 1, wherein the conjugate linker is connected to the 5′ end of the oligonucleotide.

23. The oligomeric compound of claim 1, wherein the conjugate linker is connected to the 3′ end of the oligonucleotide.

24. The oligomeric compound of claim 8, wherein the conjugate linker is connected to a carboxy terminus of the peptide extender.

25. The oligomeric compound of claim 8, wherein the conjugate linker is connected to an amino terminus of the peptide extender.

26. The oligomeric compound of claim 8, wherein the peptide extender comprises an amino acid sequence that is at least 70%, at least 75%, at least 80%, at least 85%, at least 90%, at least 95%, or 100% identical to:
KPPPAGSSPG (SEQ ID NO: 48), X₁PPPAGSSPG (SEQ ID NO: 49), X₁X₂PPAGSSPG (SEQ ID NO: 50), X₁P X₂PAGSSPG (SEQ ID NO: 51), X₁PP X₂AGSSPG (SEQ ID NO: 52), X₁PPP X₂GSSPG (SEQ ID NO: 53), X₁PPPA X₂SSPG (SEQ ID NO: 54), X₁PPPAG X₂SPG (SEQ ID NO: 55), X₁PPPAGS X₂PG (SEQ ID NO: 56), X₁PPPAGSS X₂G (SEQ ID NO: 57), and X₁PPPAGSSPX (SEQ ID NO: 58), wherein X₁ is selected from lysine, 2-azido-acetyl lysine, D-lysine, L-lysine, N⁶-(2-azido-acetyl)-D-lysine, and N⁶-(2-azido-acetyl)-L-lysine; and X₂ is any amino acid.

27. (canceled)

28. The oligomeric compound of claim 8, wherein the peptide extender comprises an amino acid sequence that is at least 70%, at least 75%, at least 80%, at least 85%, at least 90%, at least 95%, or 100% identical to:
CPPPAGSSPG (SEQ ID NO: 59), XPPPAGSSPG (SEQ ID NO: 49), CXPPAGSSPG (SEQ ID NO: 60), CPXPAGSSPG (SEQ ID NO: 61), CPPXAGSSPG (SEQ ID NO: 62), CPPPXGSSPG (SEQ ID NO: 63), CPPPAXSSPG (SEQ ID NO: 64), CPPPAGXSPG (SEQ ID NO: 65), CPPPAGSXPG (SEQ ID NO: 66), CPPPAGSSXG (SEQ ID NO: 67), and CPPPAGSSPX (SEQ ID NO: 68), wherein X is any amino acid.

29. (canceled)

30. (canceled)

31. The oligomeric compound of claim 8, wherein the peptide extender has an amino acid sequence selected from: XPAPSGPSPG (SEQ ID NO: 81), XAGSIKPPPAGSSPG (SEQ ID NO: 82), and XAGMSGASAG (SEQ ID NO: 83), wherein X is selected from lysine, cysteine, and N⁶-(2-azido-acetyl)-lysine.

32.-105. (canceled)

106. The oligomeric compound of claim 1, wherein the conjugate linker comprises maleimide.

107. The oligomeric compound of claim 1, wherein the conjugate linker and the oligonucleotide are connected via click chemistry.

108.-114. (canceled)

115. The oligomeric compound of claim 1, wherein the oligonucleotide consists of 12 to 30 linked nucleosides.

116. The oligomeric compound of claim 1, wherein the oligonucleotide is a modified oligonucleotide comprising at least one of a modified internucleoside linkage, a modified sugar moiety, and a modified nucleobase.

117. (canceled)

118. The oligomeric compound of claim 116, wherein the modified sugar moiety is a non-bicyclic sugar moiety selected from a 2′-MOE sugar moiety, a 2′-OMe sugar moiety, a 2′-NMA sugar moiety, or a 2′-F sugar moiety.

119. The oligomeric compound of claim 116, wherein the modified sugar moiety is a bicyclic sugar moiety comprising a 2′-4′ bridge selected from —O—CH₂—; and —O—CH(CH₃)—.

120. (canceled)

121. (canceled)

122. The oligomeric compound of claim 116, wherein the oligonucleotide has
a 5′-region consisting of 1-6 linked nucleosides;
a deoxy region consisting of 6-10 linked nucleosides; and
a 3′-region consisting of 1-6 linked nucleosides; wherein
each of the 5′-region nucleosides and each of the 3′-region nucleosides comprises a modified sugar moiety.

123.-128. (canceled)

129. The oligomeric compound of claim 116, wherein each internucleoside linkage is independently selected from a phosphorothioate internucleoside linkage and a phosphodiester internucleoside linkage.

130. (canceled)

131. (canceled)


SEQUENCE LISTING (83 sequences)

SEQ ID NO: 1 - DNA, 13001 bp, Homo sapiens
attactccctcattaatacaatataaaaattatttacgttgtattaggtattataagcaaactagagatgatttaagggatatgggaggatatctgtaggttacataaggaacttgagcatcttcagattttagcatccgaggagaggttttagaaccaaggccccatggataccaagggatgactgtacaacattctggcggctgggcacagtggctcaagcctgtaatcccagcactttgggaggccgaggcgggcggatcacgaggtcaggagatccagaccatcctggttaacacggtgaaaccctgtctctgctaaaactacaaaaaattagtcgggcgtggtggcgggcgcctgtagtaccagctactcgggaggctgaggcaagtgaattgcttgaaccagggaggcggaggttacggtgagccgagatcccgccactgcactccagcctcagcgatagagtgagacttcgtctcaaaaaaaaaaaaaaagaaacattctgtaatctgtcaaggtaatggaaattaatgcagtgacactgacaataatttaaagcattttagggctcttaacgttcatctcactcgctctagagtgaaggagagagcagagagtacagagcaggtcctaggtcctcccccagatatagaaaaaccgcagttggagagactgacagaagtgctggagaggaagggaggcagcaggacgtggaaaatctgggattcaagaacagcttctgctattaattagctgtgtgtcatttcaggcaaatcacaaaatctccaggcccccaaatccccatttgcgaaacgaggtgacctgcctcagttttccttcatccagcgataaaattctactaaaaaatttccaaccccagagcaagcatcaggactgtgcctgaagggatccggctgcatctcagtaatattccactcttacaggcttaaaaataaaaaagaaaagaaaagaaaacaagaacagtttcaccagcgtcaattgagaacagctgcttcaacaggccctgctttatgtgggcaggtggggacagggagttggcccagaaaacaggaccctcatttcctggagcccccagggcagctccccaacaccgtacacaacctgcatcatttacaggagccaaaggagttttagaaatcaacttcatagagttgctgtatcattgtgttagttttgccatcctaacctatacagcgtcactaatctctcccctcggagttgactgcctaaaaacaagccatggatacaggttccaaagacccgggggtgagggagcggagagggtgggtgtcaccaccccgtccagctccaagctttgtgtgccctggaactctccattttaggtcattgcttcagtttcttttctaaaaaattaggctgctgcaaggtcagcctgagaccacttctgccccgagaattctagactagtaagacctggtgacatacaaacgacgaagatattttacaataatgccatggcccttgatagctacacgaggtttgtgttctgattttaaattaatggatgacgtcgagactatatggaggaaatgacaaaggacaggagagaggtgggaaaggaagacctagactgaaaatggaagttgggcagcagctccacgaaagaaagaccagcccccaagtgcagtgacagcgcagagtagcgaccgagaagttcccaggccagctgccaccccgcccccatgccattccccagaacaggcacaggcgttagggcggggcgcgcgtgcgcagtcacgcgctgcgccaaccgccacagctccgggaaggcggccaggaccggctagagccggttagaaccagtggcgcccgcccacgagccagcgcctcacaaagggagggcggctcacggccctcgcgtatccctgcgcggcgctcgcgagccgcccctcccccggcgtttgtccctgacgcagccccaccggttgcgcagtccctccccgcccccgctctcccctccgcagcctgcagcccgagacttctgtaaaggactggggccccgcaactggcctctcctgccctcttaagcgcagcgccattttagcaacgcagaagcccggcgccgggaagcctcagctcgcctgaaggcaggtcccctctgacgcctccgggagcccaggtttcccagagtccttgggacgcagcgacgagttgtgctgctatcttagctgtccttataggctggccattccaggtggtggtatttagataaaaccactcaaactctgcagtttggtcttggggtttggaggaaagcttttatttttcttcctgctccggttcagaaggtctgaagctcatacctaaccaggcataacacagaatctgcaaaacaaaaacccctaaaaaagcagacccagagcagtgtaaacacttctgggtgtgtccctgactggctgcccaaggtctctgtgtcttcggagacaaagccattcgcttagttggtctactttaaaaggccacttgaactcgctttccatggcgatttgccttgtgagcactttcaggagagcctggaagctgaaaaacggtagaaaaatttccgtgcgggccgtggggggctggcggcaactggggggccgcagatcagagtgggccactggcagccaacggcccccggggctcaggcggggagcagctctgtggtgtgggattgaggcgttttccaagagtgggttttcacgtttctaagatttcccaagcagacagcccgtgctgctccgatttctcgaacaaaaaagcaaaacgtgtggctgtcttgggagcaagtcgcaggactgcaagcagttgggggagaaagtccgccattttgccacttctcaaccgtccctgcaaggctggggctcagttgcgtaatggaaagtaaagccctgaactatcacactttaatcttccttcaaaaggtggtaaactatacctactgtccctcaagagaacacaagaagtgctttaagaggtattttaaaagttccgggggttttgtgaggtgtttgatgacccgtttaaaatatgatttccatgtttcttttgtctaaagtttgcagctcaaatctttccacacgctagtaatttaagtatttctgcatgtgtagtttgcattcaagttccataagctgttaagaaaaatctagaaaagtaaaactagaacctatttttaaccgaagaactactttttgcctccctcacaaaggcggcggaaggtgatcgaattccggtgatgcgagttgttctccgtctataaatacgcctcgcccgagctgtgcggtaggcattgaggcagccagcgcaggggcttctgctgagggggcaggcggagcttgaggaaaccgcagataagtttttttctctttgaaagatagagattaatacaactacttaaaaaatatagtcaataggttactaagatattgcttagcgttaagtttttaacgtaattttaatagcttaagattttaagagaaaatatgaagacttagaagagtagcatgaggaaggaaaagataaaaggtttctaaaacatgacggaggttgagatgaagcttcttcatggagtaaaaaatgtatttaaaagaaaattgagagaaaggactacagagccccgaattaataccaatagaagggcaatgcttttagattaaaatgaaggtgacttaaacagcttaaagtttagtttaaaagttgtaggtgattaaaataatttgaaggcgatcttttaaaaagagattaaaccgaaggtgattaaaagaccttgaaatccatgacgcagggagaattgcgtcatttaaagcctagttaacgcatttactaaacgcagacgaaaatggaaagattaattgggagtggtaggatgaaacaatttggagaagatagaagtttgaagtggaaaactggaagacagaagtacgggaaggcgaagaaaagaatagagaagatagggaaattagaagataaaaacatacttttagaagaaaaaagataaatttaaacctgaaaagtaggaagcagaagaaaaaagacaagctaggaaacaaaaagctaagggcaaaatgtacaaacttagaagaaaattggaagatagaaacaagatagaaaatgaaaatattgtcaagagtttcagatagaaaatgaaaaacaagctaagacaagtattggagaagtatagaagatagaaaaatataaagccaaaaattggataaaatagcactgaaaaaatgaggaaattattggtaaccaatttattttaaaagcccatcaatttaatttctggtggtgcagaagttagaaggtaaagcttgagaagatgagggtgtttacgtagaccagaaccaatttagaagaatacttgaagctagaaggggaagttggttaaaaatcacatcaaaaagctactaaaaggactggtgtaatttaaaaaaaactaaggcagaaggcttttggaagagttagaagaatttggaaggccttaaatatagtagcttagtttgaaaaatgtgaaggactttcgtaacggaagtaattcaagatcaagagtaattaccaacttaatgtttttgcattggactttgagttaagattattttttaaatcctgaggactagcattaattgacagctgacccaggtgctacacagaagtggattcagtgaatctaggaagacagcagcagacaggattccaggaaccagtgtttgatgaagctaggactgaggagcaagcgagcaagcagcagttcgtggtgaagataggaaaagagtccaggagccagtgcgatttggtgaaggaagctaggaagaaggaaggagcgctaacgatttggtggtgaagctaggaaaaaggattccaggaaggagcgagtgcaatttggtgatgaaggtagcaggcggcttggcttggcaaccacacggaggaggcgagcaggcgttgtgcgtagaggatcctagaccagcatgccagtgtgccaaggccacagggaaagcgagtggttggtaaaaatccgtgaggtcggcaatatgttgtttttctggaacttacttatggtaaccttttatttattttctaatataatgggggagtttcgtactgaggtgtaaagggatttatatggggacgtaggccgatttccgggtgttgtaggtttctctttttcaggcttatactcatgaatcttgtctgaagcttttgagggcagactgccaagtcctggagaaatagtagatggcaagtttgtgggttttttttttttacacgaatttgaggaaaaccaaatgaatttgatagccaaattgagacaatttcagcaaatctgtaagcagtttgtatgtttagttggggtaatgaagtatttcagttttgtgaatagatgacctgtttttacttcctcaccctgaattcgttttgtaaatgtagagtttggatgtgtaactgaggcgggggggagttttcagtatttttttttgtgggggtgggggcaaaatatgttttcagttctttttcccttaggtctgtctagaatcctaaaggcaaatgactcaaggtgtaacagaaaacaagaaaatccaatatcaggataatcagaccaccacaggtttacagtttatagaaactagagcagttctcacgttgaggtctgtggaagagatgtccattggagaaatggctggtagttactcttttttccccccacccccttaatcagactttaaaagtgcttaaccccttaaacttgttattttttacttgaagcattttgggatggtcttaacagggaagagagagggtgggggagaaaatgtttttttctaagattttccacagatgctatagtactattgacaaactgggttagagaaggagtgtaccgctgtgctgttggcacgaacaccttcagggactggagctgcttttatccttggaagagtattcccagttgaagctgaaaagtacagcacagtgcagctttggttcatattcagtcatctcaggagaacttcagaagagcttgagtaggccaaatgttgaagttaagttttccaataatgtgacttcttaaaagttttattaaaggggaggggcaaatattggcaattagttggcagtggcctgttacggttgggattggtggggtgggtttaggtaattgtttagtttatgattgcagataaactcatgccagagaacttaaagtcttagaatggaaaaagtaaagaaatatcaacttccaagttggcaagtaactcccaatgatttagtttttttccccccagtttgaattgggaagctgggggaagttaaatatgagccactgggtgtaccagtgcattaatttgggcaaggaaagtgtcataatttgatactgtatctgttttccttcaaagtatagagcttttggggaaggaaagtattgaactgggggttggtctggcctactgggctgacattaactacaattatgggaaatgcaaaagttgtttggatatggtagtgtgtggttctcttttggaatttttttcaggtgatttaataataatttaaaactactatagaaactgcagagcaaaggaagtggcttaatgatcctgaagggatttcttctgatggtagcttttgtattatcaagtaagattctattttcagttgtgtgtaagcaagtttttttttagtgtaggagaaatacttttccattgtttaactgcaaaacaagatgttaaggtatgcttcaaaaattttgtaaattgtttattttaaacttatctgtttgtaaattgtaactgattaagaattgtgatagttcagcttgaatgtctcttagagggtgggcttttgttgatgagggaggggaaactttttttttttctatagacttttttcagataacatcttctgagtcataaccagcctggcagtatgatggcctagatgcagagaaaacagctccttggtgaattgataagtaaaggcagaaaagattatatgtcatacctccattggggaataagcataaccctgagattcttactactgatgagaacattatctgcatatgccaaaaaattttaagcaaatgaaagctaccaatttaaagttacggaatctaccattttaaagttaattgcttgtcaagctataaccacaaaaataatgaattgatgagaaatacaatgaagaggcaatgtccatctcaaaatactgcttttacaaaagcagaataaaagcgaaaagaaatgaaaatgttacactacattaatcctggaataaaagaagccgaaataaatgagagatgagttgggatcaagtggattgaggaggctgtgctgtgtgccaatgtttcgtttgcctcagacaggtatctcttcgttatcagaagagttgcttcatttcatctgggagcagaaaacagcaggcagctgttaacagataagtttaacttgcatctgcagtattgcatgttagggataagtgcttatttttaagagctgtggagttcttaaatatcaaccatggcactttctcctgaccccttccctaggggatttcaggattgagaaatttttccatcgagcctttttaaaattgtaggacttgttcctgtgggcttcagtgatgggatagtacacttcactcagaggcatttgcatctttaaataatttcttaaaagcctctaaagtgatcagtgccttgatgccaactaaggaaatttgtttagcattgaatctctgaaggctctatgaaaggaatagcatgatgtgctgttagaatcagatgttactgctaaaatttacatgttgtgatgtaaattgtgtagaaaaccattaaatcattcaaaataataaactatttttattagagaatgtatacttttagaaagctgtctccttatttaaataaaatagtgtttgtctgtagttcagtgttggggcaatcttgggggggattcttctctaatctttcagaaactttgtctgcgaacactctttaatggaccagatcaggatttgagcggaagaacgaatgtaactttaaggcaggaaagacaaattttattcttcataaagtgatgagcatataataattccaggcacatggcaatagaggccctctaaataaggaataaataacctcttagacaggtgggagattatgatcagagtaaaaggtaattacacattttatttccagaaagtcaggggtctataaattgacagtgattagagtaatactttttcacatttccaaagtttgcatgttaactttaaatgcttacaatcttagagtggtaggcaatgttttacactattgaccttatatagggaagggagggggtgcctgtggggttttaaagaattttcctttgcagaggcatttcatccttcatgaagccattcaggattttgaattgcatatgagtgcttggctcttccttctgttctagtgagtgtatgagaccttgcagtgagtttatcagcatactcaaaatttttttcctggaatttggagggatgggaggagggggtggggcttacttgttgtagctttttttttttttacagacttcacagagaatgcagttgtcttgacttcaggtctgtctgttctgttggcaagtaaatgcagtactgttctgatcccgctgctattagaatgcattgtgaaacgactggagtatgattaaaagttgtgttccccaatgcttggagtagtgattgttgaaggaaaaaatccagctgagtgataaaggctgagtgttgaggaaatttctgcagttttaagcagtcgtatttgtgattgaagctgagtacattttgctggtgtatttttaggtaaaatgctttttgttcatttctggtggtgggaggggactgaagcctttagtcttttccagatgcaaccttaaaatcagtgacaagaaacattccaaacaagcaacagtcttcaagaaattaaactggcaagtggaaatgtttaaacagttcagtgatctttagtgcattgtttatgtgtgggtttctctctcccctcccttggtcttaattcttacatgcaggaacactcagcagacacacgtatgcgaagggccagagaagccagacccagtaagaaaaaatagcctatttactttaaataaaccaaacattccattttaaatgtggggattgggaaccactagttctttcagatggtattcttcagactatagaaggagcttccagttgaattcaccagtggacaaaatgaggaaaacaggtgaacaagctttttctgtatttacatacaaagtcagatcagttatgggacaatagtattgaatagatttcagctttatgctggagtaactggcatgtgagcaaactgtgttggcgtgggggtggaggggtgaggtgggcgctaagcctttttttaagatttttcaggtacccctcactaaaggcaccgaaggcttaaagtaggacaaccatggagccttcctgtggcaggagagacaacaaagcgctattatcctaaggtcaagagaagtgtcagcctcacctgatttttattagtaatgaggacttgcctcaactccctctttctggagtgaagcatccgaaggaatgcttgaagtacccctgggcttctcttaacatttaagcaagctgtttttatagcagctcttaataataaagcccaaatctcaagcggtgcttgaaggggagggaaagggggaaagcgggcaaccacttttccctagcttttccagaagcctgttaaaagcaaggtctccccacaagcaacttctctgccacatcgccaccccgtgccttttgatctagcacagacccttcacccctcacctcgatgcagccagtagcttggatccttgtgggcatgatccataatcggtttcaaggtaacgatggtgtcgaggtctttggtgggttgaactatgttagaaaaggccattaatttgcctgcaaattgttaacagaagggtattaaaaccacagctaagtagctctattataatacttatccagtgactaaaaccaacttaaaccagtaagtggagaaataacatgttcaagaactgtaatgctgggtgggaacatgtaacttgtagactggagaagataggcatttgagtggctgagagggcttttgggtgggaatgcaaaaattctctgctaagactttttcaggtgaacataacagacttggccaagctagcatcttagcggaagctgatctccaatgctcttcagtagggtcatgaaggtttttcttttcctgagaaaacaacacgtattgttttctcaggttttgctttttggcctttttctagcttaaaaaaaaaaaaagcaaaagatgctggtggttggcactcctggtttccaggacggggttcaaatccctgcggcgtctttgctttgactactaatctgtcttcaggactctttctgtatttctccttttctctgcaggtgctagttcttggagttttggggaggtgggaggtaacagcacaatatctttgaactatatacatccttgatgtataatttgtcaggagcttgacttgattgtatattcatatttacacgagaacctaatataactgccttgtctttttcaggtaatagcctgcagctggtgttttgagaagccctactgctgaaaacttaacaattttgtgtaataaaaatggagaagctctaaattgttgtggttcttttgtgaataaaaaaatcttgattggggaaaaaagatgggtgttctgtgggcttgttctgttaaatctgtggtctataaacacagcacccataattacagcataatcttcaagtagggtacggactttgggggattggtgcgagggtagtgggtgagtggcctactaaaaagcccagtaacccccacaggaaaatagggaacttctttttaagtagcctcctttccactatttagtaattggctgtgagctgggctgggggagaaatggggcggggtgtgtgtgtcattggaaagctctcttttttgtttttttgagacagtctcactttgtcccccaggctggagtgtagtggcatgatctctgcaaactgcaacctccacttctggggtccaagtggttgtcctgcttcaccctccctgtagctgggactacaggtgcacaccaccacgcctggctaatttttgtattttcagttagagacgtggttttaccatattggccaggctggtctcaaactcctgacctcgtgtgatccacccgcctgggcctctgaaagtgctgggattacaggtgtgagccaccaagcctggccgatccttttaagtttttaaaccagttaagctctttggttccccctcagagtcccaaggtcctgggtcactcaggatcacattttcctttaatcagttgtcactggtccccttttgtccctttgaacgtgctgtgggattagtagcagcatctggctgttggaaggactggctgggatctcaggtgaaataccctccctggccctccttacccttaagatctccctgaaaagtcagaccttgcttattggagtttgagatgcattctagtaaacccagacctaccagagagagggacccacgtctgaagaatcttccagcctggggaacctgtgactggccacacccttagcctaatggaggggcctcagaagcagaactacctttttttttttttttaatggtattacattttgcaaatggagaggtagactctgattgggccccttttgacaacgcagcagagctgaggtttgaatcctagtcagtcaacacttcaacccctatgttgctgtttcttcaatgtctagaatgttctgatagatgaagcaatggcccggcaggtcagcagcacaccaacccccaacctacccagcctgtataggccttggccacaggagctttctcttcccttgaagtgatctaatgggggatggggtatctgtctcgctggttatgaaagaagctgagctaggtgctctcctaagttgcttaagtgtcttctgaattgatgcccagtcagtttcgtatggagaaaggtctctgtttttccaaagcagaaggttctgtggaagcacaaatactttactggagagcaaaggagtacgagtaggaattgggggccacgcacagtggctcacgcctataatcccaggactttgggaggccgaggtgggcagatcacctgaggtcaggagttcgagaccagcctgaccaacatggagaaaccccatctctactaaaaatacaaaattagccgggcgtggtggcgtatgtctgtaaccccagctattcgagaggctgaggtaggagaatcgcttggacccgggaggcggaggttgcagtgagctgagattgcgccactgcactccagcctgggcaacaagagcgaaactgtctcaaaaaaaaaaaaaaaagaagaagaaaggctgggcacggtggctcaagcctgtaatcccagcactttgggaggccgaggcgggcggatcacgaagtcaggagattgaaaccatcctggctaacacggtgaaaccccgtctctactacacacacacacacacacacacacacacacacacacacacacacaatttgctgggcgtggtggagggcgcctgtagtcctagctactcgtgaggctgaggcaggagaatggcgtgaacctgggaggcggagcttgccgtgaggtgagatcgggccactgcactccagcctgggcgacagagcgagactccgtcttaaaaaaaaaaaaaaaaaaaaaaagagtaggagctgggcattcgccaggggctacgtagttcctggagttgaggaagttctccaggattgaaaaatgtaaagatcagacactgatcacagcaacaggaggaggcagaaaagtaggccggaagtcatttccaccctctgagcacaaaatggagagcataaattccccaaaagc

SEQ ID NO: 2 - DNA, 20 bp, Artificial sequence
  Synthetic oligonucleotide
ccaggctggttatgactcag

SEQ ID NO: 3 - protein, 13 aa, Artificial sequence
  Synthetic peptide
  MISC_FEATURE (1)..(1): Xaa is selected from lysine or 2-azido-acetyl lysine
XLYENKPRRPYIL

SEQ ID NO: 4 - protein, 12 aa, Artificial sequence
  Synthetic peptide
  MISC_FEATURE (5)..(5): Xaa is N6-(2-azidoacetyl)-lysine
LYENXPRRPYIL

SEQ ID NO: 5 - protein, 22 aa, Artificial sequence
  Synthetic peptide
  MISC_FEATURE (1)..(1): Xaa is selected from lysine or 2-azido-acetyl lysine
XPPPAGSSPGLYENKPRRPYIL

SEQ ID NO: 6 - protein, 22 aa, Artificial sequence
  Synthetic peptide
  MISC_FEATURE (1)..(1): Xaa is selected from lysine or 2-azido-acetyl lysine
XPAPSGPSPGLYENKPRRPYIL

SEQ ID NO: 7 - protein, 27 aa, Artificial sequence
  Synthetic peptide
  MISC_FEATURE (1)..(1): Xaa is selected from lysine or 2-azido-acetyl lysine
XAGSIKPPPAGSSPGLYENKPRRPYIL

SEQ ID NO: 8 - protein, 22 aa, Artificial sequence
  Synthetic peptide
  MISC_FEATURE (1)..(1): Xaa is selected from lysine or 2-azido-acetyl lysine
XAGMSGASAGLYENKPRRPYIL

SEQ ID NO: 9 - DNA, 16 bp, Artificial sequence
  Synthetic oligonucleotide
gactcagtattggtcg

SEQ ID NO: 10 - DNA, 12 bp, Artificial sequence
  Synthetic oligonucleotide
gactcagtattg

SEQ ID NO: 11 - DNA, 8 bp, Artificial sequence
  Synthetic oligonucleotide
gactcagt

SEQ ID NO: 12 - DNA, 4 bp, Artificial sequence
  Synthetic oligonucleotide
gact

SEQ ID NO: 13 - protein, 22 aa, Artificial sequence
  Synthetic peptide
CPPPAGSSPGLYENKPRRPYIL

SEQ ID NO: 14 - DNA, 19 bp, Artificial sequence
  Synthetic oligonucleotide
tcagcattctaatagcagc

SEQ ID NO: 15 - DNA, 16 bp, Artificial sequence
  Synthetic oligonucleotide
gcattctaatagcagc

SEQ ID NO: 16 - DNA, 20 bp, Artificial sequence
  Synthetic oligonucleotide
gactcagtattggtcggacc

SEQ ID NO: 17 - DNA, 20 bp, Artificial sequence
  Primer
ctcctcagaccgctttttgc

SEQ ID NO: 18 - DNA, 24 bp, Artificial sequence
  Primer
taacctggttcatcatcgctaatc

SEQ ID NO: 19 - DNA, 20 bp, Artificial sequence
  Probe
ccgtcatgccgacccgcagt

SEQ ID NO: 20 - DNA, 15 bp, Artificial sequence
  Primer
tcgccgcttgctgca

SEQ ID NO: 21 - DNA, 17 bp, Artificial sequence
  Primer
atcggccgtgatgtcga

SEQ ID NO: 22 - DNA, 23 bp, Artificial sequence
  Probe
ccatggtcaaccccaccgtgttc

SEQ ID NO: 23 - DNA, 10001 bp, Mus musculus
tgactatcagcctgtatggtcattaagttctgtctaccctgggaaagcctctgcccaaacctccctattcacaggtcctagaacgtagaggtggggagcaggacggtgccgccaggccgtgtgcgatcgcgagctctggatctcaatgcgccccggggcgctgtttcccacgactccagcagcttttctaaaaatccaggcagcctccagtttacgggatcaacccgagactcgcttccctttgaaaattctagagtataaagtaaacgtacgagcaaagtatgtgtcttaacacttaatggatgacatagagaccaaaaaagccatgtccgtgggcccagtaggccgcaataaggggcgaccaggaaactgcagcacagccccccgcagccgccctgctcccacaccagtcattccagcaccgtggtgaaggcgcttgggggcggggcggggcgcgcctgcgcagcgaggctctgcagcagaaactttgcctagaccggctggaaccggttagaaccggtcgaacccggccggctgccagccctcgattcagcagctcacaaagggaggcggcgactcacgacccgcgtatccttgcgcctctccccaccccctttgtcctcgcgacgggttccgcggtcctccccgccctccctggcgcggcccccgctttctgcgcccagtgacgctttctccatggtcctgggagaaagagaaaaacatcctttcccctccgtcgtagttttaggaagcgatgagatagacctggggaccttgccgccacgggccgggctctgacggttattagcgcagtgcgggtggtgctcggcatggccgccaaggtcgccgtgccctcacctgcagcgaccatggccttgctgggctgagaccgcagcctaacatggcggacgtaggcaagcaccaaagcgctcgtgtacccgggctcggaaaagtggccccgagagcagccggaggctgcaggtcgtccctacaggagcattcccagtataaaccagtacaaagtgtcaccacctcagaagccactcgcagggccggtcactttccgagagacctccatcttgtttcgcatgaaatggcagccgctcggggagttacaaaatgggaagtggaagctgaagctgtgggaaagcctgttttaacacttgcaacatacgctataccctctgtcctcccaggaaaacgcaaaaggtgttgaaacatctgaaaaacttggggctcccatttttaatagctattagttcatgttttttctccttgtgaccagaaattttaaacctatttgtacctatttagctggtacaagctaaacatttctctgtattagcaaggtccaagaggcccacacgacgtcaagaaaaatctagaaacttggaagtcaggatctatttttaactctctgaggaactatttttcttccttcaccaaggtggtggagggttactaggttccggtggagtgacgtgtccctttgcaataaataccggcgctccgggctctgcgtcaggcattcaggcagcgagagcagagcagcgtagagcagcacagctgagctcgtgaggcaggagactcagcccgaggaaatcgcagataagtttttaattaaaaagattgagcagtaaaaagaattagaactctaaacttaagctaatagagtagcttatcgaaatattacttagtcttaataatctaagaagatcttaagagataacatgaaggcttatttaaacagtttgaaaaaggaaatgaggagaaaagtatttgtactgtataatggaggctgaccagagcagtttaggagattgtaaagggaggttttgtgaagttctaaaaggttctagtttgaaggtcggccttgtagattaaaacgaaggttacctaaatagaatctaagtggcatttaaaacagtaaagttgtagagaatagtttgaaaatgaggtgtagttttaaaagattgagaaaagtaggttaagttgacggccgttataaaaatccttcgactggcgcatgtacgtttgaaggcatgagttggaaacagggaagatggaagtgttaggctagccgggcgatggtggcgcacgcctttaatcctagcacttgggaggcagaggcaggcggatttctgagttcgaggccagcctggtctacagagtgagttccaggacagccagggctacacagagaaaccctgtcttgaaaaaacaaaaaggttaggctagtatttggagaaagaagattagaaaatggaagtgaaagacgaagaagacatacaggaaggtgaagaaaaagctgttagagaagataggaaaatagaagacaaagcatctttagaagacagaaaaggtacttaaaggcacaggtagtaggaagccgaagaatagaagatagaaagaagcaagatagaaaaacaaaatggaagttaagacaactttggatgccagcattcaagataggcaaagaagataagattgaggccaaaaggttggataagatataaagtcagaaggaaattatctttaaagccataagttcaaatttctgatggagcgagcagtttagaagagtctttagacagccacatacaagattgaagctagcaatcaaagctactaggactgaagtaaaaagttaaggcagaatgcctttgaagagttagaagaatattaaaagccttaacttgtagcttaattttgcttgatgacaaaaggacttttgataacagtttcaagattgtcagcattttgcattggacttgagctgaggtgcttttaaaatcctaacgactagcattggcagctgacccaggtctacacagaagtgcattcagtgaactaggaagacaggagcggcagacaggagtcccgaagccagtttggtgaagctaggaaggactgaggagccagcagcagcagtgcatggtgaagatagcccaggaaagagtgcggttcggtggaggaagctaggaagaaggagccatacggatgtggtggtgaagctgggaaagggttccaggatggtggagcgagagcgagttggtgatgaagctagctggcggcttggcttgtcaactgcgcggaggaggcgagcaggcattgtggagaggatagatagcggctcctagaccagcatgccagtgtgcaagaaaggctgcagggagagcatgcggtgcggtaacattccttgaggtcggcaacatggtggtggttttctgtaacttggatggtaacttgtttactttgtcttaatagttatgggggagttgtaggcttctgtgtaaagagatatatctggggctgtatgtaggcctttgcgggtgttgtaggtttttctttttcagggttatgtcctcttgcatcttgtcagaagcttttgagggctgactgccaaggcccagaaagaagaatggtagatggcaagttgtctttaaccgctcagaggggaatgaatggtagagccagcacaacctcccagttttgtaagacgttgtagtttgaacagatgacctaccacaagcctcactcctgtgtaggggaggtaattgggcaaagtgcttttgggggaatgggggcaaaatatattttgagttcttttccccttaggtctgtctagaatcctaaaggcagatgactcaagggaaccagaaaaaaggaaatccactctcaggataagcagagctcgccaggtttacagtttgtaggaagtagaggatggatgctagctttcacactgagtgtggaggagctggccatggcggaattgctggtagtttactctttccccctcccttaatgagatttgtaaaatcctaaacacttttacttgaaatatttgggagtggtcttaacagggaggagtgggtgggggaaacgttttttttctaagattttccacagatgctatagttgtgttgacacactgggttagagaaggcgtgtactgctatgctgttggcacgacaccttcagggactggagctgccttttgtccttggaagagttttcccagttgccgctgaagtcagcacagtgcggctttggttcacagtcacctcaggagaacctcaggagcttggctaggccagaggttgaagttaagttttacagcaccgtgatttaaaatatttcattaaaggggaggggtaaaacttagttggctgtggccttgtgtttgggtgggtgggggtgttaggtaattgtttagtttatgatttcagataatcataccagagaacttaaatatttggaaaaacaggaaatctcagctttcaagttggcaagtaactcccaatccagtttttgcttcttttttcctttttctttttttgaggcgggcagctaaggaaggttggttcctctgccggtccctcgaaagcgtagggcttgggggttggtctggtccactgggatgatgtgatgctacagtggggactcttctgaagctgttggatgaatatagattgtagtgtgtggttctcttttgaaatttttttcaggtgacttaatgtatcttaataactactataggaacaaaggaagtggctttaatgaccctgaaggaatttcttctggtgatagcttttatattatcaagtaagagatactatctcagttttgtataagcaagtctttttcctagtgtaggagaaatgattttccttgtgactaaacaagatgtaaaggtatgctttttttcttcttgtgcattgtatacttgtgtttatttgtaacttataatttaagaattatgataattcagcctgaatgtcttttagagggtgggcttttgttgatgagggaggggaaacctttttttttctgtagacctttttcagataacaccatctgagtcataaccagcctggcagtgtgatgacgtagatgcagagggagcagctccttggtgaatgagtgataagtaaaggcagaaaaaataatgtcatgtctccatggggaatgagcatgagccagagattgttcctactgatgaaaagctgcatatgcaaaaatttaagcaaatgaaagcaaccagtataaagttatggcaatacctttaaaagttatggcttatctaccaagctttatccacaaaagtaaagaattgatgaaaaacagtgaagatcaaatgttcatctcaaaactgcttttacaaaagcagaatagaaatgaagtgaaaatgctgcattaagcctggagtaaaaagaagctgagcttgttgagatgagtgggatcgagcggctgcgaggcggtgcagtgtgccaatgtttcgtttgcctcagacaggtttctcttcataagcagaagagttgcttcattccatctcggagcaggaaacagcagactgctgttgacagataagtgtaacttggatctgcagtattgcatgttagggatagataagtgccttttttctctttttccaaaaagacctgtagagctgttgaatgtttgcagctggcccctcttaggcagttcagaattttgagtagttttcccatccagcctcttaaaaattcctaagccttgcaccgatgggctttcatgatgggatagctaataggcttttgcatcgtaaacttcaacacaaaagcctacatgattaatgcctactttaattacattgcttacaagattaaggaatctttatcttgaagaccccatgaaagggatcattatgtgctgaaaattagatgttcatattgctaaaatttaaatgtgctccaatgtacttgtgcttaaaatcattaaattatacaaattaataaaatacttcactagagaatgtatgtatttagaaggctgtctccttatttaaataaagtcttgtttgttgtctgtagttagtgtgggcaattttggggggatgttcttctctaatcttttcagaaacttgacttcgaacacttaagtggaccagatcaggatttgagccagaagaccgaaattaactttaaggcaggaaagacaaattttattctccatgcagtgatgagcatttaataattgcaggcctggcatagaggccgtctaactaaggactaagtaccttaggcaggtgggagatgatggtcagagtaaaaggtaactacatattttgtttccagaaagtcaggggtctaatttgaccatggctaaacatctagggtaagacacttttcccccacatttccaaatatgcatgttgagtttaaatgcttacgatcatctcatccactttagccttttgtcacctcacttgagccacgagtggggtcaggcatgtgggtttaaagagttttcctttgcagagcctcatttcatccttcatggagctgctcaggactttgcatataagcgcttgcctctgtcttctgttctgctagtgagtgtgtgatgtgagaccttgcagtgagtttgtttttcctggaatgtggagggagggggggatggggcttacttgttctagctttttttttacagaccacacagaatgcaggtgtcttgacttcaggtcatgtctgttctttggcaagtaatatgtgcagtactgttccaatctgctgctattagaatgcattgtgacgcgactggagtatgattaaagaaagttgtgtttccccaagtgtttggagtagtggttgttggaggaaaagccatgagtaacaggctgagtgttgaggaaatggctctctgcagctttaagtaacccgtgtttgtgattggagccgagtccctttgctgtgctgccttaggtaaatgtttttgttcatttctggtgaggggggttgggagcactgaagcctttagtctcttccagattcaacttaaaatctgacaagaaataaatcagacaagcaacattcttgaagaaattttaactggcaagtggaaatgttttgaacagttccgtggtctttagtgcattatctttgtgtaggtgttctctctcccctcccttggtcttaattcttacatgcaggaacattgacaacagcagacatctatctattcaaggggccagagaatccagacccagtaaggaaaaatagcccatttactttaaatcgataagtgaagcagacatgccattttcagtgtggggattgggaagccctagttctttcagatgtacttcagactgtagaaggagcttccagttgaattgaaattcaccagtggacaaaatgaggacaacaggtgaacgagccttttcttgtttaagattagctactggtaatctagtgttgaatcctctccagcttcatgctggagcagctagcatgtgatgtaatgttggccttggggtggaggggtgaggtgggcgctaagcctttttttaagatttttcaggtacccctcactaaaggcactgaaggcttaatgtaggacagcggagccttcctgtgtggcaagaatcaagcaagcagtattgtatcgagaccaaagtggtatcatggtcggttttgattagcagtggggactaccctaccgtaacaccttgttggaattgaagcatccaaagaaaatacttgagaggccctgggcttgttttaacatctggaaaaaaggctgtttttatagcagcggttaccagcccaaacctcaagttgtgcttgcaggggagggaaaagggggaaagcgggcaaccagtttccccagcttttccagaatcctgttacaaggtctccccacaagtgatttctctgccacatcgccaccatgggcctttggcctaatcacagacccttcacccctcaccttgatgcagccagtagctggatccttgaggtcacgttgcatatcggtttcaaggtaaccatggtgccaaggtcctgtgggttgcaccagaaaaggccatcaattttccccttgcctgtaatttaacattaaaaccatagctaagatgttttatacatagcacctatgcagagtaaacaaaccagtatgggtatagtatgtttgataccagtgctgggtgggaatgtaggaagtcggatgaaaagcaagcctttgtaggaagttgttggggtgggattgcaaaaattctctgctaagactttttcaggtggacataacagacttggccaagctagcatcttagtggaagcagattcgtcagtagggttgtaaaggtttttcttttcctgagaaaacaaccttttgttttctcaggttttgctttttggcctttccctagctttaaaaaaaaaaaagcaaaagacgctggtggctggcactcctggtttccaggacggggttcaagtccctgcggtgtctttgcttgactcttatatcatgaggccattacatttttcttggagggttctaaaggctctgggtatggtagctgatatcactggaacactccccagcctcagtgttgaactcttgataattaactgcattgtctttcaggttatgcccaattcgtcttattacctctgagtcgacacacctcctactatttattgaatactttgattttatgaaataaaaactaaatatctctcattgtgtgcttctttgtgcataaaacacaggcttattttaagcctaaagagaccaaatgtctgatctacctcagcttctccgattagtgaggccttccctgtttccttgggctgcatggctctttcatgcagatggctctaaagttgggcttgggtcctaggtggccactcttgcacctcaggaacacaaggcctttccctgctgttcaggctctcctccctgagaaaacattctggattgtctatgaggaagttgggaaaagatggtgtcgaaaagaggtggtgtgcattgctcctctgttcctaacactggatggaagactagttttcatgtagtttagggaaatagttatacatggtctaaaggcccaaaaacattcccagagtgtatgcaatactgtgtgtaagtgtgcactgcgtgtgtttggaggtcagaacttctctgaggttctagagatgaagcaagtcctcagccatggcccaagaatgggaaggaactgggtcctgctgtaccacttcccattccttaaggaacagtttggcccggtgtggtgcaagcatggtcggtcactgaaaaaagaaaacccacttaggtttcacaggcttgaagagctgcatgtcatccagcaaattactggctgctgtaaggacaggcccctaggtcccagtcccaggtgcccttcctgccactcaatcaagccttacaccctgggcaaaaacatcctgcgttgaaggttcagctcccagggctggaaacttgtgctggcatctaccccagttcaaaggggctcagcacattgacaactaaaactaagccctcaggtgagcaaaatggtctccttaaggcaatcatggtcattggtgttcctgcagtaaaggacagcatcacagctgatgtctgtgtactggctagttttgtatcaacttgacacagctggaattatcacagagaaagcttcagttggggaagtgcctccaagagatcctccacgagatcctgctctaaggcattttctcaattagtgatcaagggggaaagaccccttgtgtgtgggaccatctctgggctggtagtcttggttcagttctataagagagcaggctgagcaagccaggggaagcaagccagtaaagaacatccctccatggcctctgcatcagctcctgcttcctgacctgcttgagttccagtcctgacttccttggtgatgaacagcagtatggaagtgtaagccgaataaaccctgtcctccccaacttgcttcttggtcatgtttgtgcaggaatagaaaccctgactaagacagtctgagacctgacagatctgtgctaaagtctggtaccaactgagctagaccctgccacacacctcagtaatggcccattctgaattcacccagagctgaggctttgccgaggtgaggcacaaagacttcactggagagcaggagatatgaacagaggttggggctcacacttcctgattgggggccagga

SEQ ID NO: 24 - DNA, 23 bp, Artificial sequence
  Primer
tgggttagagaaggcgtgtactg

SEQ ID NO: 25 - DNA, 18 bp, Artificial sequence
  Primer
tcagcggcaactgggaaa

SEQ ID NO: 26 - DNA, 25 bp, Artificial sequence
  Probe
cgttggcacgacaccttcagggact

SEQ ID NO: 27 - DNA, 20 bp, Artificial sequence
  Primer
ggcaaattcaacggcacagt

SEQ ID NO: 28 - DNA, 20 bp, Artificial sequence
  Primer
gggtctcgctcctggaagat

SEQ ID NO: 29 - DNA, 27 bp, Artificial sequence
  Probe
aaggccgagaatgggaagcttgtcatc

SEQ ID NO: 30 - RNA, 22 nt, Artificial sequence
  Synthetic oligonucleotide
uaaaaucuacagucauaggaau

SEQ ID NO: 31 - DNA, 1435 bp, Homo sapiens
ggcggggcctgcttctcctcagcttcaggcggctgcgacgagccctcaggcgaacctctcggctttcccgcgcggcgccgcctcttgctgcgcctccgcctcctcctctgctccgccaccggcttcctcctcctgagcagtcagcccgcgcgccggccggctccgttatggcgacccgcagccctggcgtcgtgattagtgatgatgaaccaggttatgaccttgatttattttgcatacctaatcattatgctgaggatttggaaagggtgtttattcctcatggactaattatggacaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtaatgaccagtcaacaggggacataaaagtaattggtggagatgatctctcaactttaactggaaagaatgtcttgattgtggaagatataattgacactggcaaaacaatgcagactttgctttccttggtcaggcagtataatccaaagatggtcaaggtcgcaagcttgctggtgaaaaggaccccacgaagtgttggatataagccagactttgttggatttgaaattccagacaagtttgttgtaggatatgcccttgactataatgaatacttcagggatttgaatcatgtttgtgtcattagtgaaactggaaaagcaaaatacaaagcctaagatgagagttcaagttgagtttggaaacatctggagtcctattgacatcgccagtaaaattatcaatgttctagttctgtggccatctgcttagtagagctttttgcatgtatcttctaagaattttatctgttttgtactttagaaatgtcagttgctgcattcctaaactgtttatttgcactatgagcctatagactatcagttccctttgggcggattgttgtttaacttgtaaatgaaaaaattctcttaaaccacagcactattgagtgaaacattgaactcatatctgtaagaaataaagagaagatatattagttttttaattggtattttaatttttatatatgcaggaaagaatagaagtgattgaatattgttaattataccaccgtgtgttagaaaagtaagaagcagtcaattttcacatcaaagacagcatctaagaagttttgttctgtcctggaattattttagtagtgtttcagtaatgttgactgtattttccaacttgttcaaattattaccagtgaatctttgtcagcagttcccttttaaatgcaaatcaataaattcccaaaaatttaaaaaaaaaaaaaaaaaaaaaa

SEQ ID NO: 32 - protein, 12 aa, Artificial sequence
  Synthetic peptide
LYENKPRRPYIL

SEQ ID NO: 33 - protein, 28 aa, Artificial sequence
  Synthetic peptide
QDRLDAPPPPAAPLPRWSGPIGVSWGLR

SEQ ID NO: 34 - protein, 44 aa, Artificial sequence
  Synthetic peptide
QDRLDAPPPPAAPLPRWSGPIGVSWGLRAAAAGGAFPRGGRWRR

SEQ ID NO: 35 - RNA, 21 nt, Artificial sequence
  Synthetic oligonucleotide
uccuaugacuguagauuuuaa

SEQ ID NO: 36-37 - skipped (2 entries)

SEQ ID NO: 38 - protein, 9 aa, Artificial sequence
  Synthetic peptide
PPPAGSSPG

SEQ ID NO: 39 - protein, 9 aa, Artificial sequence
  Synthetic peptide
  misc_feature (1)..(1): Xaa can be any naturally occurring amino acid
XPPAGSSPG

SEQ ID NO: 40 - protein, 9 aa, Artificial sequence
  Synthetic peptide
  misc_feature (2)..(2): Xaa can be any naturally occurring amino acid
PXPAGSSPG

SEQ ID NO: 41 - protein, 9 aa, Artificial sequence
  Synthetic peptide
  misc_feature (3)..(3): Xaa can be any naturally occurring amino acid
PPXAGSSPG

SEQ ID NO: 42 - protein, 9 aa, Artificial sequence
  Synthetic peptide
  misc_feature (4)..(4): Xaa can be any naturally occurring amino acid
PPPXGSSPG

SEQ ID NO: 43 - protein, 9 aa, Artificial sequence
  Synthetic peptide
  misc_feature (5)..(5): Xaa can be any naturally occurring amino acid
PPPAXSSPG

SEQ ID NO: 44 - protein, 9 aa, Artificial sequence
  Synthetic peptide
  misc_feature (6)..(6): Xaa can be any naturally occurring amino acid
PPPAGXSPG

SEQ ID NO: 45 - protein, 9 aa, Artificial sequence
  Synthetic peptide
  misc_feature (7)..(7): Xaa can be any naturally occurring amino acid
PPPAGSXPG

SEQ ID NO: 46 - protein, 9 aa, Artificial sequence
  Synthetic peptide
  misc_feature (8)..(8): Xaa can be any naturally occurring amino acid
PPPAGSSXG

SEQ ID NO: 47 - protein, 9 aa, Artificial sequence
  Synthetic peptide
  misc_feature (9)..(9): Xaa can be any naturally occurring amino acid
PPPAGSSPX

SEQ ID NO: 48 - protein, 10 aa, Artificial sequence
  Synthetic peptide
KPPPAGSSPG

SEQ ID NO: 49 - protein, 10 aa, Artificial sequence
  Synthetic peptide
  misc_feature (1)..(1): Xaa can be any naturally occurring amino acid
XPPPAGSSPG

SEQ ID NO: 50 - protein, 10 aa, Artificial sequence
  Synthetic peptide
  MISC_FEATURE (1)..(1): Xaa is an amino acid selected from selected from lysine, 2-azido-acetyl lysine, D-lysine, L-lysine, N6-(2-azido-acetyl)-D-lysine, and N6-(2-azido-acetyl)-L-lysine
  misc_feature (2)..(2): Xaa can be any naturally occurring amino acid
XXPPAGSSPG

SEQ ID NO: 51 - protein, 10 aa, Artificial sequence
  Synthetic peptide
  MISC_FEATURE (1)..(1): Xaa is an amino acid selected from selected from lysine, 2-azido-acetyl lysine, D-lysine, L-lysine, N6-(2-azido-acetyl)-D-lysine, and N6-(2-azido-acetyl)-L-lysine
  misc_feature (3)..(3): Xaa can be any naturally occurring amino acid
XPXPAGSSPG

SEQ ID NO: 52 - protein, 10 aa, Artificial sequence
  Synthetic peptide
  MISC_FEATURE (1)..(1): Xaa is an amino acid selected from selected from lysine, 2-azido-acetyl lysine, D-lysine, L-lysine, N6-(2-azido-acetyl)-D-lysine, and N6-(2-azido-acetyl)-L-lysine
  misc_feature (4)..(4): Xaa can be any naturally occurring amino acid
XPPXAGSSPG

SEQ ID NO: 53 - protein, 10 aa, Artificial sequence
  Synthetic peptide
  MISC_FEATURE (1)..(1): Xaa is an amino acid selected from selected from lysine, 2-azido-acetyl lysine, D-lysine, L-lysine, N6-(2-azido-acetyl)-D-lysine, and N6-(2-azido-acetyl)-L-lysine
  misc_feature (5)..(5): Xaa can be any naturally occurring amino acid
XPPPXGSSPG

SEQ ID NO: 54 - protein, 10 aa, Artificial sequence
  Synthetic peptide
  MISC_FEATURE (1)..(1): Xaa is an amino acid selected from selected from lysine, 2-azido-acetyl lysine, D-lysine, L-lysine, N6-(2-azido-acetyl)-D-lysine, and N6-(2-azido-acetyl)-L-lysine
  misc_feature (6)..(6): Xaa can be any naturally occurring amino acid
XPPPAXSSPG

SEQ ID NO: 55 - protein, 10 aa, Artificial sequence
  Synthetic peptide
  MISC_FEATURE (1)..(1): Xaa is an amino acid selected from selected from lysine, 2-azido-acetyl lysine, D-lysine, L-lysine, N6-(2-azido-acetyl)-D-lysine, and N6-(2-azido-acetyl)-L-lysine
  misc_feature (7)..(7): Xaa can be any naturally occurring amino acid
XPPPAGXSPG

SEQ ID NO: 56 - protein, 10 aa, Artificial sequence
  Synthetic peptide
  MISC_FEATURE (1)..(1): Xaa is an amino acid selected from selected from lysine, 2-azido-acetyl lysine, D-lysine, L-lysine, N6-(2-azido-acetyl)-D-lysine, and N6-(2-azido-acetyl)-L-lysine
  misc_feature (8)..(8): Xaa can be any naturally occurring amino acid
XPPPAGSXPG

SEQ ID NO: 57 - protein, 10 aa, Artificial sequence
  Synthetic peptide
  MISC_FEATURE (1)..(1): Xaa is an amino acid selected from selected from lysine, 2-azido-acetyl lysine, D-lysine, L-lysine, N6-(2-azido-acetyl)-D-lysine, and N6-(2-azido-acetyl)-L-lysine
  misc_feature (9)..(9): Xaa can be any naturally occurring amino acid
XPPPAGSSXG

SEQ ID NO: 58 - protein, 10 aa, Artificial sequence
  Synthetic peptide
  MISC_FEATURE (1)..(1): Xaa is an amino acid selected from selected from lysine, 2-azido-acetyl lysine, D-lysine, L-lysine, N6-(2-azido-acetyl)-D-lysine, and N6-(2-azido-acetyl)-L-lysine
  misc_feature (10)..(10): Xaa can be any naturally occurring amino acid
XPPPAGSSPX

SEQ ID NO: 59 - protein, 10 aa, Artificial sequence
  Synthetic peptide
CPPPAGSSPG

SEQ ID NO: 60 - protein, 10 aa, Artificial sequence
  Synthetic peptide
  misc_feature (2)..(2): Xaa can be any naturally occurring amino acid
CXPPAGSSPG

SEQ ID NO: 61 - protein, 10 aa, Artificial sequence
  Synthetic peptide
  misc_feature (3)..(3): Xaa can be any naturally occurring amino acid
CPXPAGSSPG

SEQ ID NO: 62 - protein, 10 aa, Artificial sequence
  Synthetic peptide
  misc_feature (4)..(4): Xaa can be any naturally occurring amino acid
CPPXAGSSPG

SEQ ID NO: 63 - protein, 10 aa, Artificial sequence
  Synthetic peptide
  misc_feature (5)..(5): Xaa can be any naturally occurring amino acid
CPPPXGSSPG

SEQ ID NO: 64 - protein, 10 aa, Artificial sequence
  Synthetic peptide
  misc_feature (6)..(6): Xaa can be any naturally occurring amino acid
CPPPAXSSPG

SEQ ID NO: 65 - protein, 10 aa, Artificial sequence
  Synthetic peptide
  misc_feature (7)..(7): Xaa can be any naturally occurring amino acid
CPPPAGXSPG

SEQ ID NO: 66 - protein, 10 aa, Artificial sequence
  Synthetic peptide
  misc_feature (8)..(8): Xaa can be any naturally occurring amino acid
CPPPAGSXPG

SEQ ID NO: 67 - protein, 10 aa, Artificial sequence
  Synthetic peptide
  misc_feature (9)..(9): Xaa can be any naturally occurring amino acid
CPPPAGSSXG

SEQ ID NO: 68 - protein, 10 aa, Artificial sequence
  Synthetic peptide
  misc_feature (10)..(10): Xaa can be any naturally occurring amino acid
CPPPAGSSPX

SEQ ID NO: 69 - skipped

SEQ ID NO: 70 - protein, 15 aa, Artificial sequence
  Synthetic peptide
CAGSIKPPPAGSSPG

SEQ ID NO: 71 - protein, 15 aa, Artificial sequence
  Synthetic peptide
KAGSIKPPPAGSSPG

SEQ ID NO: 72 - protein, 13 aa, Artificial sequence
  Synthetic peptide
ELYENKPRRPYIL

SEQ ID NO: 73 - protein, 13 aa, Artificial sequence
  Synthetic peptide
  MISC_FEATURE (1)..(1): Xaa is selected from lysine or 2-azido-acetyl lysine
XLYENKPKRPYIL

SEQ ID NO: 74 - protein, 13 aa, Artificial sequence
  Synthetic peptide
  MISC_FEATURE (1)..(1): Xaa is selected from lysine or 2-azido-acetyl lysine
XLYENKPRKPYIL

SEQ ID NO: 75 - protein, 13 aa, Artificial sequence
  Synthetic peptide
  MISC_FEATURE (1)..(1): Xaa is selected from lysine or 2-azido-acetyl lysine
XLYENKPKKPYIL

SEQ ID NO: 76 - protein, 45 aa, Artificial sequence
  Synthetic peptide
CQDRLDAPPPPAAPLPRWSGPIGVSWGLRAAAAGGAFPRGGRWRR

SEQ ID NO: 77 - protein, 29 aa, Artificial sequence
  Synthetic peptide
CQDRLDAPPPPAAPLPRWSGPIGVSWGLR

SEQ ID NO: 78 - DNA, 20 bp, Artificial sequence
  Synthetic oligonucleotide
ctgagtcataaccagcctgg

SEQ ID NO: 79 - DNA, 20 bp, Artificial sequence
  Synthetic oligonucleotide
attcactttcataatgctgg

SEQ ID NO: 80 - DNA, 28070 bp, Homo sapiens
ccacaaatgtgggagggcgataaccactcgtagaaagcgtgagaagttactacaagcggtcctcccggccaccgtactgttccgctcccagaagccccgggcggcggaagtcgtcactcttaagaagggacggggccccacgctgcgcacccgcgggtttgctatggcgatgagcagcggcggcagtggtggcggcgtcccggagcaggaggattccgtgctgttccggcgcggcacaggccaggtgaggtcgcagccagtgcagtctccctattagcgctctcagcacccttcttccggcccaactctccttccgcagcctcgggacagcatcaagtcgatccgctcactggagttgtggtccgcgtttttctacgtcttttcccactccgttccctgcgaaccacatccgcaagctccttcctcgagcagtttgggctccttgatagcgttgagtggaggccctgccgcgacttggcagtagcttattttgttcactcctctctggctggtgtgggggaggtgggggcattaggccagggtgaagcaggggaaccacttaggagtctgttaagatgatctgaacttcagaacaagatgttattaacagagtgaaagtatttggattctgggtatattttgaaatcggaggcaacaggtttttcagatagattcgataacggaggttatcctgaatagttgaaaagataaagttgccttttgctgaggtgggaaagagaagattgccagtagagcaggtttctcaggagttcagtcttgggcatagcatggtaggggtgaatttggctggagtgagttggagagtaggagaagagaaatccaaggcaacatttgaccagcctgggcaacatagtgtgactccgagtctgcaaaaattagacgggtgttgtggtgcgcgtctgtggtctcagctacctggaaggttcaggccttggaaggctcagggaggtggaggctgcagtgatctgtgattgcgcctctgcactccagcctgggcgacagagccagaccctgtcttaaaacaaaataaacggccgggcgcggtggctcaagcctgtaatcccagcactttgggaggccgaggcggccggatcacaaggtcaggagatcgagaccatcctggctaacacggtgaaaccccgtctctactacaaatacaaaaaattagccgggcgtggtgacgggcgcctgtagtcccagctactcgggaggctgaggcaggagaatgtcatgaagccgggaggcggagcttgcagtgagccgagatcgcgccactgcactccagcctgggcgatagagcaagactccgtctcaaataaataaataaataaataaataaataataaaaacatcggtaggcatatttcaaggaattctatttaaaaaaaatttttttagagacaagttcgctctctgtggcccaggctggagtacagtggcatgatcctagcccatggcagcgttgatctcttggcctcaagcgaccctcctttggagtcgctgggcctaaaggagtgagccaccacgaaattttattataaatggagggtagagaaattgggcaataaatggagggggaagtgagttaagaggaattttaattatgtgtgtgtggttttaaaagaggggggtcttgctctgttgcccaggctgctggggtgccagtggcgcaatcatgaatcactacagccttggactcctggcctcaagctatcctcccacctctgcctcccaaagtactgggattactagtgtgagccactgcactaagataggagcaacatgtttcagcatgtttgtgggttgataggaaagatgagaatgggaaagttgatgtcggaaagaagacaatggctagagcaatgtcctagagtaggtaagaagggatggatttggcctttgttggaaacattagcggttcttttggtgacagctatatagttaacacatctatgatacgtgaatgggcagataggatggcaggagattttgaaagttctcttgattcttactgttctcttagtgaaagaagcaaggttatcagctagaagctgggatgggagaggaaagagaagatgggaagtagatagttctttagaagagtgggcaagggttggactagggaagtttagtggaaatattgctaggcaacataaagagcctacttgagattcgtggtcatgagttgaaggagaccagacagcaagattgtgtatgagggcacccacagagtaaatggagagttgaaattaatgcagttgtgattttaccacgtggatatgaagaagtgagggggagaagtacaaaggagttctcttaatgattgaccatggaatttaagctggctaagaaaggaagtgagaggccgggcgcggtggctcacgcctgtaatcccagcactttgggagactgaggtgggtggattacctgaggtcaggagtttgagaccaacctggccgatatggcgaaaccccatctctaataaaaatacagaaaaattagccgggaatggtggcaggtgcctgtaatcccagctactcaagaggctgtggcaggagtatcccttggacccaggaggtggaggttgcagtgagccgagatcacgccactgtactccagcctggacgatatagtgagacttcacctcaaaaaaaaaaaaaaagaaaggaagtgaggattttaagaccctgagagacagtttaaaaagtgggaggatcggccgggcgctgtggctgacacctgtaatcccagcactttgggaggccgagttgggcagatcacaaggtcaggagttcgagaccagcctggccaatatggtgaaaccttgtctctactaaaaatacaaaaattagccgggcatggtgtcacgtgtctataatcccagctactcgggaggctgaggcagaaaaattgcttgaacctgggaggcagaggttgcagacagctgagatcactccattgcactccagcctgggcaacaagagcaaaactttgtctttaaaaaaaaaaaaaaaaaaagaatacaaaaattagccgggcgtggtggcgcgtgcctataatcccagctacttgggaggctgaggcaggagaatcagttgaacacgggaggcgaggtttgcagtgagccgagattgcgccactgcactccagcctgggcgacagagcaggactcctcttggaaaaaaaaaattagctgggcatggtggcaggtgcctgtagtctcagctactagggaggctgaggcaggaaaatcacttgaacccgggatgtggagtttgcagtgacccgagatcgtgccactgtactccatcctgggcgacaaaatgagactctgcctcaaaaaaaaaaaaaaaaaaaagtgggaggatcaatgtactgccagtcctaatgaagtggaatgattgtccccatcaaatcactagtaggagtaagttgcagagcctagaaggtgatggttaagagagtgggattcttgaaactgcatttatggagaggttgtggttattggttataataaataaatacagttgaagtgagtgagtagctgagatttggggatgtatcagttcattcttacactgctacaaagacatacctgagaccaggtatttataaagataagaggtttaatcagctcacagttctgctgcctgtacaggcttctcttgtggaggcctaaggaaacttacagtcatggtggaaggtgaaggggaaacaagcacagtcttcacatggccagcaggagagagagagaagggggaagtgctacatactttaaaacaaccagatcttgtgagaacgcttatcaggaaacagcacttggggatggtgctaaatcattagaaatcacccccatgatccagtcgcctcctaccatgcccacctccaacactggggatcacaattcagcatgagatttgggtaggaacacagagctgcaccacatcagaggatgtacaagattgtggtggagaggagtttagagacctgcaaatatagggtaattgaagggatcatctacatggatatttaaatcaccaaaaattatgacaggagtagtgttggagagagaactgcgatgtaaacattaaggaatgaggaagagtgactcggtaggctgtaggtgactgcaataggaaacgataatagactgtgagtctggtgacaagattttccttctttctttttttccccccccccgagacagggcctctttttgttgcccaggtgggagtgcagtggcgcgatcacggctcactacaacctcctcccaagctcaagggattctcccacttcagcctctcaagtagctggaactacaggtgctgaccaccatgcctggctactttttgtcaggattttcaaggctgggaattttgagaggggaatggaggagaataatctgaaagtgcaagtaaggagcagggaagatttcttttttcttttttttttttttttttgagtcggagtctggctcagtcgcccaggctggagtgcagtggcgagatctccgctcactgcaagctccgcctcccgtgttcacgccattctcctccttcagcctcccgagtagctgggactacaggcgcccgccaccacgcccagctaattgtttttttgtatttttagtagagacggggtttcaccgtgttagccaggatggtctcaatctcctgactttgtgatccgcccaccccggcctcccaaagcgcttgggattacaggcgtgagccaccgcgccagccagagcagggaagatttcttccccacatctccagtaggtacagtgatatgaagtgtgtggaggagaaaagaggaaacatctatcatttgagatggctgcgaaaggaaaaggcatcctcagggagctagattttacttagagcaagaaatgaagggatgattcagaggttaaaagagtggattttatgaattactcaagggagcacagtggaagtttcaggaagtggtaggagaaggtagaagatggcagggtgttgggaataatttgagaaatctgagctactggaaatgactgagaatcagatataaaggcagtcctggtggtccgttctggctgccgttgctgtgtaacgaatctgccaaaacttagtggcttgaaacaacaaagaacattttattatctctcattgtttctgtgggttaggaatttgtgagagccgtgctgggcagttttcgtgcggctgtctcgtggttgcacctacatagttgctagagctacagtagctggggactgagcagctagggattggcaggctatctctttttttcatgtagtctcatgaagatttctttatgtggtttcaatgtgtgggctggtttggatttccttatagcatggtggcctcagttggattgctgttttgtgatccttttcatccctccttgtcctgtccccagacaaccactgatctactttctgtcaccatagattagcctgcatttttaagaatttttataaacgtggaatgatagagtaccttttttgtcacgtttcttttatttatcatagctattttgattttcatccattttattgctgagtagtatcccattgcatgtatatactatactgtattcattcgcttgcttgtgaacatttgggctttttccagtttgggactgttaacaagtagagccactatgaatattagtgtataagacttcatatagccaaggctggcagatcgcttgagcccaggagtttgagaccagcctgggaaacatggtgaaacctctatttttattttaaaatcaaaaattaaaaattttctataaaaaattttaaagaagactttgtatagacatacgctttcatttttcttgagtgaatacttaggtctcagggtagatgtattttaagtctttaaggagctgtcaaactcttcctcaaagtggtggttgtaccatgttactttttaatataacagagattaattgagcaaagaaaaattcaaaagttggacagcccccacaactaaataggttcagaacagctcccccattttgcattttgaccagcaatgtatgaaagttccatttgctcagtgtccctgcaaacacctggtatggtcagtctttttaattttaggcattataatagatatagtggcttcttgtgattttaattagcatttcctaatgaccagtgctgctgttgatcatttcatgagtgtatttgccatccgtatatcttttttggtgaagtgtctattcaaatcatttgggttttttttttttttgttttttttttttggagacagtgtctcactctgtcacccaggctgttgtgcagtggtgcaatcacacagcctactgcagcctccacctcctgcgctcagtcttcttgtctcagccttctgagtagctgaaattacgagcacacgccacaatgcctggctaattttttaaaattttgtagaaacaaggtctcattatgttgcctgggcttgtcgtgaactcctgggctcaagcaatcttcctgcctcagcctcccaaagattgggattgcaagtatgagccactgcacccggccaacttacccatcttttaattgaatttttttgttgttgaggtttgagagttcttcatgtttgctgggtacaatatctttatcagataggtaacttgcatgtattttctcccggtttacactttggtttttcattttgttaacaacgtctttttaagaacagaaaatcttaattttgctgaaatctaatttttcagttttttctttgatggttttgagagaggaggtaaaaaaagactaggtaagccgatagttagacagagtcctcggtagaacttcccttctaacaaaaagcagcccaagaaatcacttctcttctaacaaggagcagcctggaagatcgggctgtaaacatgtataaggaagcagctctggcacagagggggagcttcctgggtaatcagcaagcttcacatacgtaaggtgggtatgtgaagtaaacacagtatgtgaagtaaacacagtggaccttagtacatactcagataaggaagctggaagcttgcatgttgtgagttgttggggttgcctgcagctgcacggagagaaaggggtacctggggccaggcatgtccaccatggtggctccacctccccttatttagcacatgcacaataggaaagagataagcaatgtggagtagctcaggccaaggacctgcctgcataataaaaggttggggtgggggatgccagagattcacgctctgtgcagatggcaacacctggtcctaactggttttttgctccctatgtgtagataagctacccccttcccattagctcatttataaaaatgcttgcatttcactgtggaatgggaactcttttcaggacctctctctgcaggagagagctagtctctttcttttgcctattaaacttctgctctagcctcacacccttggtgtgtcagcgtccttgatttcctcagcgtgagaccaagaacctcgggtgccaccccaggcaacaaggccatttcagtttgttcttttgttataggcaatccatgatcacagatttttctctcttttttttttttacacagtttagagttttagttttacacttaggtctgtaatccattttgtattaattcttatatgtggctcagtgtaggtggaaatttggtttgtttttgcataaggatttccaatagttttaccaccatttcttgaaactactatgctttctctattaaaccacatttgtaactttagttaaaatcagtcacatatatcacagggctatttctgactctcaattctgttacattgtctattagtgtatattgatgtcagtactacacttttaattactattgcttcagggtatgtcttgtaaaccaaaaataaaattataggccccccccgcccctgcacaaccaactgaatggacccatcctctcagccaagggcattccaaaattaacctgaaaaactagttcaagccatgatgggaagggggagttggacatgtctcatcacaccctactaccttttggaattactgatagaacagactcttaaagtctgaaaagaaacatttacaacctaccctctctgaagcctgctacctgggagcttcatctgcatgataaaaccttggtctccacaaccccttatggtaacccaaacattcctttctgttgataataactctttcaactagttgccaattagaaaatctttaaatcttcctatgacctagaaacctccctacccccactttgagttgtcctgcctttcctgacagaactcatgtacatcttacatatattgattgatgcctcatgtctccctaaaatgtataaaacaaagctgtaccccaccaccttggggacatgtcatcaggacctcctgtggctgtgtcataggagcgtctttaactttggcaaaataaactttctaaattgattgaaacctgtcttagctacttctggtttacagtcttaaagttagataatgtaaattgtccagctttggtttatttttgtccttagtagttccatataaattttagaatcagcttttcaatttaatacactactttcctcttagatccacaattaaatatatttgatgctaacaattctgttttatgtttttcgttttttttttttgagacaagagtttcgctcttgttgcccaggctggagtgcagtggcgcgatcttggctcaccacaacctccacctcccaggttcaagcaattcttctgcctcagcctcccgagtagctgggattacaggcatgcgccaccacgcccggctaattttgtatttttagtagagacggggtttcaccatgttgatcaggctggtcttgaactcctgacctcaggtgatccacccacctcggcctcccaaagtgttgggattacaggcgtgaaccaccatgcctggccagttctgttatttttaaaacccaagtttccctggtcatatcttggttggatgaagcgtattttcaatagattaccctggaaaggctagtgagtacggtattcttctacattttagacttttcttagtcttgctacttcaaggacagctaggctgcatataaaattcttggctcatactttttccccataaatttctatgagaaagtctaatgataactgattttctttattttgtaacttagtctttttgcttagaggctctctgaggatgggagggggttcttcctcccatccctaggaatttttcttttttttaaattcctaatcactagaccaccaggaagattgtttgttttgttttgtttttattcttcagggaccccatttatacatacgttaaataaatactgtttgccaatgtatcaaccattttgcttcttatttatttttgttcctttggttctttttcatggctttgctttggtgctccttagattttcagtcagatgtatttgtccttgggtaccttgtaatcagtattaccttttcttctgtcgctttgttttctgttcgttttgaaattacttgtttcctggtctggcaataacagttgagatatgaggagtttgagctgccatctgtctatgtatcttgctttaagactgcactcttctattgatatcactggccttgattttgtgatttctttatttcttcaggaccacccttcattttctactgtttgcttcctttttttttgagatggagtctcactctgtcactcaggctggagtgcagtgatcttggctcattgcaacctctgcctcccgggttccagcaattctcctgcctcagcctcccaagtatctgggactacaggtgtgcaccaccatgcccggctaagttttgtatttttaatagagacggggttttgccacattggcaggctggtctcaaactcctgatgtcaagtgatccacccaccccacccacctctgcatcccaaagtgctgggattacaggaatgagctgccgtgcccagcctcccccctacccccctttttttctttcgagacagagattataggtgtgagccactggacccagcctgtttttattccttttaccaaatctccaaggaatatcttcccttccaagtgcgaatgtaaccttaagtcagttaacctctttgtgattacttttcttatctgcaaagtgacttaatgatcttaagtactttttttttttgagacagggtctcactgtcaccctggctggagtgcagtggcacgatctctgatctccactcactgcaatctcctcttccctggttcaagcggccctcccaccttagccttctgggtagctgggactacagatgtgaaccaccacgcccagctaatttttgtactttttgtagagatggggttttgccatgttgcccaggctgggattattaagtactttttatcatacagcaagattgacattttatattggaatacatttgtctctatataacggagattaacaggaaaatgacaagcctgggtgcggtggctcatgcctgtaatcccagcactttgggaggctgaggtgggaggatcacttgaggtcaggagttcgagaccagttttgccaagatgatgaaagcccatgtctactaaaaatacaaaaattagcccagcttgatggtgggcgcctataatcccagctatttgagagactgaggcaggagaatcacttgaacctgggcagcagaggttgcagtgagccgagatcatgccactgcactccagcctgggtggcatagcgagactcttgtctcaagagaaaacaaaacaaaacaaaaaaaaaacaggaaaatgacaaaaagtaatattacaactcagtgaattttataacaaacttttttggaattcattgactaatactataccaaatccaaaatactctctagtataccaaatccaactctaccctatagtataaattggattctatttggacttgtctcactaatccctcatacagtgtgttttattttttattgaagtaaaaaaatttgtcattttaaccatttttaagtatatagttcagtaatattaagtatgttcatgttgttgcgcaatagatcttcggaagtttttcgtcttgcaacctgaaactctacccattagcaaattcccatttctccttacacttagcccttggtaatcatcattcttttttttttttttttgagatggagttttactcttgttgcccaggctggagtgcaatggtgcaatctcgactcaccacaacctccgcctcccaggttcaagcaattctacctcagcctcccgagtagctgggattacagtcatgcaccaccacgcccggctaattttgtatttttagtagagaaggggtttctccatgttgaggctggtctcgaactcctgacctcaggtgatctgcccacctcggcctcccaaagtgctgggattacaggcgtgagccactgcgcctggcccattctttctaattctataaatttgactacttagttaccttacataaataaattcttatagttagtgttatttttgcttccatgccttttttgttgttgttcatgctcttacttggaatgcgttctattttgtctacctatgcacatcctgttgggttttttttttttttgggggttttttttgtttttttttgtttttttttcccagacaaggtctcaatttgttacccaggctggagtgcagcggcgccatctccactcactgcatcctcaacttcctgggcccaggtgatcctctcgcctcagcccctgcaggtagctgggactataggcatgtgccaccatgcccagctaaatttggtttttttgtttgtttgtttttgagacagagtctcactctgtcacccaggctggagtgcagtggcacaatctcagctcactgcaatctctgccgcccgggttcaagtgattctcctgcctcagcctcccaagcagctgggattacaggtgactgccaccacgccagctaagttttgtagttttagtagagatggggtttcaccttgttggccatgctggtctcgaactcctgacctcgtgatctgcctgcttctgcctcccaaagtgctggaattacaggcatgagccaccacgcccggccagaatttttgtatttttagtagacacaaggttcttaccctgttgcctaggctggtctggaagtcctggactcaagcaattcacctgccttggcctcccaaaatgctgggattacaagccaccatgcccggcctaaatcctgttgttttgttttgttttattttgttttgttttgttttgtttgttttttgagacagagtctcgctatgtctctcaggctgtagtgcagtggcgcgatcttggctcactgccacctctgcctcccaggttcaagtgattctcctgcctcagcctcccaagtagctgggattacaggcatgtgctactatgtccggctaatttttgtatttttagtagagacagggtttcaccatgttggccaggctggtctcgaactcctgacctcgtgatccacccacctcggccacccaaagtgctgggattacaggcgtgagtggtttttatttcttaggccggtttcctccatatgatcttgcagtagacattaatttctttcctttttaattaaaatactgtttgtatttcacattttgatgtttgttaagatttgttttatattgttttttgttttgtcttgtgtgatagtcttaaatccctagttagataataactggagagtaccatgtttctatatatctctcagtgacttgcacagtgctagcagatagtgctaaaaaattatttattattattattattttgttattgttgttgttgttgttagacagggtcttcctctgtcacccaggctagagggcaatgggatgatcatagcttactgcagcctccaacaactgggctcatgtaattctcctgcctcagcttcccaagtagctgggattacaggcatgagccaccatgtctggacaaaaatatttccaggtgcagtggctcatgcctgtaattcccacacttgggaggccgagcgaggctggaggatcacttgagcctaggagttcaagaccagcttggctaagatggcgagaccccgtccctacaaaaaattttaaaaactagccaggcatggtggcatgcacctatattcccaactactcagtgggctgaggtgggagggtcatttgaacacaggaatttgaggggagaaaaaaagaagagagaaagagaagtgaaggaaggaagaaaggaaggagggagggagagaagaaagaaacgaaagaaaggaaaagaaaaggaaggaaagaaaattggtaccaggaaagcaggaaagggaaatggaagtaaaaaaataataataataataaaatgaaaattggttagtcactattaacaatttgtatccttataatctggaaacattataatttcaaaagaaaaaatattctttggatcataggttctgaggtcagaacagcattcccgtagtctagatgaagtcaagttttatctgatcttaattgaaataaatatagctggccttgaacaaatctactcatggtatgtggataggaattaaattgtaggggcattcacttgatggcattcattcttagaacatttacctatgtctagcttttggagtaaagtcacataacctctaaccaggtaagtttcctgtggctttatttaggattttaaatactcattttcagtgtaattttgttatgtgtggattaagatgactcttggtactaacatacattttctgattaaacctatctgaacatgagttgtttttatttcttaccctttccagagcgatgattctgacatttgggatgatacagcactgataaaagcatatgataaagctgtggcttcatttaaggtatgaaatgcttgcttagtcgttttcttattttctcgttattcatttggaaaggaattgataacatacgataaagtgttaaagtacatgttattcagttttcattttgaagattagatggtagtatgagttagttaaatcaggtgatatcctcctttagaagttgatagcctatatatgtcatcctttgtggaggcaatttaaataaaatttaaaacatttattcctggctgggtatggtggctcactcctgtaatcccagcactttgagaggctgaggcgggtggatcacctgaggtcaggagtttgagaccagcctggccaacatggtgaaaccccgtctttactaaaaatacaaaaattagccaagcatggtggcacgtgcctgtaatcccagctgcttgggacactgaggcaggagaattgcttgaacctggggggcagaggttgcaatgattgcaccactgcactccagcctgggcgatagagtgagactccatctcagaaaacgaacaaacaatgtattccttttagtatttttacattgtatcaaactatggaagtcctctaattgagattaataagaaaaagacaatctgaattataattttaaacatttaacaagcatgtagtaaaataatgatgaagataaatagcattagtacagcaattaatatttgtagcatgctgacagtgctctgtgtgcgtttcatatattaaattactctaatcatcccaaatcctgtaagttgggtatcaattcaagtgttcctattgggtaggaatatacagttcttttaggaaatgtagtatggttctgtgtctcaaacaggacacttacacagttggccaacatcatcaccttctccattctctgagatgtttagtcttactgagcactaaatatgggtcatcaatagtccagactaccttgagcaaacaatagtccagactaccttgagcaaacagagcatatactcatacagtgtataaagagcaccaagcatacagatttcatgtctttctcatagttactcttgtaacatgagctaaagatcagacctctatgtcacctttgtaactgatttctagatttttttttttttttgagatggggtcttgccctgtcacccaggctggagtgtagtggcgtgatcatgcctcattggagccttcaactcatgagctcaaacaatcctcctacctcagcttcctgagtagttgggaccacaggtgtgtgccaccacacccagctcatttttgtattctttgtagagatgcagtctcaccctgttgcccacgctggcctggaactcctgagctcaaaagatccctccgccttgaccttccaaagtgctgggattacaagcatgaaccactgcacccggcctagatttttaaatgtgctttccagtatacactgaaactagaagtcgactaaagaattaccaagagaattctataaaatagagattgaaatggggctcgatgtgggatgggttggtgatattgcagggagaagtaatctgagtaaaggaggaaaagaactgatttgggaaaacgatagttttagtagtgagtttgagtatgaattaagttgagattgaatttgaattaagttgaggttgaatatgaattaagttgaggttgagtttgaggtatgaattaagatgtgaaattgatcattggaaatgttagattgagaaaagtcacagctggattaatagcttcagaagtgtgtttgcagacagttgcaactaaagtaataagaatagatggccttggccgggcgcggtggctcacgcctgtaatcccagtactttgggaggctgaggcgagcaaatcacgaggtcaggagttcaagaccagcctggcccacatggtgaaaccccgtctttattaaaaatacaaaaattagctgtgcacagtggtgcacgcctgtaatcccagctactcgggaggctgagacaggagaatcgcttgaacctgggaggtggaggttgcagtgagctgagatcagtgtgactgcactccagcccggtgacagagtgagactctgtgtaaaaaaataaaataaataaaataatggccgtaagcaagtaaagaaggatggccagctcttattgggaatgcctaaatctaaggcttgatcagaagtaatgaaaccgttggggccctacattgctatgacatccaaagggccatgaatatcaggaagaaagataattaacagggtctaatgttacagagaggttgagagcaaggagatttgattaaaagggtctttagagctgatgtcaggtgtatgatgcctttaagagcagtttttatagtgcagggggtggtcaaaagagaaaataggtgctttctgaggtgacggagccttgagactagcttatagtagtaactgggttatgtcgtgacttttattctgtgcaccaccctgtaacatgtacatttttattcctattttcgtagcatgctctaaagaatggtgacatttgtgaaacttcgggtaaaccaaaaaccacacctaaaagaaaacctgctaagaagaataaaagccaaaagaagaatactgcagcttccttacaacaggttattttaaaatgttgagatttaacttcaaaggatgtctcattagtccttatttaatagtgtaaaatgtctttaacttaagtgattagtacagtgtttctattgacatatacttatacaacttcaaaaacaactattaaattttctgttatttaggaacatgcatattagtcatgaaagtataaagaattagatgggaatgataaatgctaaaatcaggacatgtgttccatttgtgaatggaaggcagggagaaggtgccgtttggaaggagtacccaagagccgtaagctgaattggcagtgttttacatcttaagctgagagatagatttttttttcccctttttctttaaaaactctaaaactgttaattccaaggaacccagaagtctaggtagattatttctgctagttaaaagcagtagtcctgaaagctgaatattttggtgtcttttgagccaactttagtttcatcattaccaagggggaagagagctaacagttgatgagcacttgctctaggccagtccagagtgctgggcaccatacgcattttatctccctcccgctattcacaacaaatatgggaggtagtttatattatagccatctaataagatggggaaactaagactcaaagagattcagaaacttgtccatgattataaatgtaagagagttggaattcagatttatgtatttagaccccaagcctttctcattacatcattttgccttccaaatctctaccctctatccttcacctccccactgatcaaaacgagatgatagtttgccctcttcaaaagaaatgtgtgcatgtatatatctttgatttcttttgtagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacaaaatgctcaagaggtaaggatacaaaaaaaaaaaaattcaatttctggaagcagagactagatgagaaactgttaaacagtatacacagttgtcagtttgatccaccgaggcattaattttttcttaatcacacccttataacaaaaacctgcatattttttctttttaaagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaaggtaaaccttctatgaaagttttccagaaaatagttaatgtcgggacatttaacctctctgttaactaatttgtagctctcccatgaaacttttgtagcttaaatacacaagaattttttgaaaaggaaataagataatgatgcaaaatagttaattttttaaaaaaatgttagacactgcagtggatgcaacaaaatactttatatgaaagatttatccagttaacttttgtggagtattaggtattagactaataattagcacacttacttaagttagaaagtataataatgcgccggacgcggtagctcacgcctgtaatcccagcactttgggaggccaaggtgggcggatcacaaggtcaggagatcgagaccatcctggctaacacggtgaaaccccatctctactgaaaatacaaaaaaatttgccgggcgtgatggcgggcacctgtagtcccagctactcgggaggctgaggcaggaggatggtgtgaaccccggaggcagagcttgcagtgagtcaagatcgtgccactgcactccaacctgggcgacagaatgagactccatctcaaacaaaaaaacaaaacaaaacaaaaaaaagtgtaataataatttatcattagctggatgatatgctgttgtttcccatgtcacctgtataagatatgtaaaataagaacacattatttacatctaatatagataaaatcctgaggcgctctcagattgttttgtagagttcaaatgtaaatattgttttcatttatggtccttttggttataagtaacagaaatcaactctaaaaagatttttattataggttagattatgtcatggaaccttaaggcttgtccctttctagttcttttgtgtaaagcggtgatttcttccatggagggaatggtatttaggcaatttttttttttttttcgagatggagtcttgctctgtcgctcaggctggagtgcagtggcaccatttcagctcactgcaacttccacctcctgggttcaagtgattctcctgcttcagcctcccaagtagctgagattacaggcacccgccaccacacccggcttattttgtatttttagtagagatggggtttcaccatgttggccaggctggtcttgaactcctgacctcaagtgatctccccaccttggccttccaaagtgctaggattacaggcgcctagcctaggcagtcattttcaaaaaacaagcatgactcaccaaaagttttaagattttctgtgataatgttcttattgaggcttacattatattacagtttcttgaatctaaaatgatgtaccctcttagaatatatacatcatgcttcattggtctcagggggctgatttttataaggagagatttgctagttttcacaatatgtcctctaagttggcatgtatagctaaacaggctttcataaaaatatacaatttagttaatgaaatttgggatatagtcttttatgattgaaataattttgctaaatagactgtctctgatttattaggtaatcaccactcttattttgttttacttccttaatgtctacatagaaaggaaatgagaaaaatccagaggttgtcatttgacttatgagtctgtttgacttcaggatttggtacatgaaatttcacttaatctttttgatatgtataaaacaaatattctgggtaattatttttatccttttggttttgagtcctttttattcctatcatattgaaattggtaagttaattttcctttgaaatattccttatagccaggtctaaaattcaatggcccaccaccgccaccgccaccaccaccaccccacttactatcatgctggctgcctccatttccttctggaccaccagtaagtaaaaaagagtataggttagattttgctttcacatacaatttgataattagcagaatagaggattgtaaaatgtcattgtagaacatcccttgggccagattctaatgggtagaaatttgaactaaacctctgggttttgtttgtttttaatgcctttctgttacccagatgcagtgctcttgtagtcccaagtctaagctctaggttgccttctttcctggcagaagttggtgtctatgccataaggaggtagttcctgttagaagggatttaattataccttatataaggaattagtgtttgcccttctaggtatagttggatgttagcttctgatgtaaactggatttctttttctttctctctctttttttttttttgttttggaggcagagttttgcccttgtaccccaggctggagtgcagtggtgtgatctcagctcacagcaacctccgcctcctgggttcaagcaattctgcctcggcctcccaagtagctgggattacaggcgactgccaccacacccggctaatttttgttttattagtagagatggggtttcaccatgttggccagactgatcttgaactcctgacctcaggtgatccacccgccttggcctcccaaagcgctgggattacaggcgtgagctgccgcacccagctgtaaactggatttctaatggtagatttttaggtattaacaatagataaaaagatactttttggcatactgtgtattgggatggggttagaacaggtgttctacccaagacatttacttaaaatcgccctcgaaatgctatgtgagctgtgtgtgtgtgtgtgtgtgtgtgtgtattaaggaaaagcatgaaagtatttatgcttgattttttttttttactcatagcttcatagtggaacagatacatagtctaaatcaaaatgtttaaactttttatgtcacttgctgtcttttcgtcctcgttaaatttaattttgttggtcttttgttgttattggttggttttctccaaatgctagctatgttaagaaatttaaggccaggtacagtggctcatgcctgtaatcccggcattttagaaggctgaggcaggaggatcacttgagctcaggagtttgagaccagtctgggcaacatagcaagacctcgtctttgtttaggggaaaaaaaagaaatttaagtaggagattatataagcaaaaatacaattaatttccagcattcactatataatataaatctccagactttacttttttgtttactggatataaacaatatctttttctgtctccagataattcccccaccacctcccatatgtccagattctcttgatgatgctgatgctttgggaagtatgttaatttcatggtacatgagtggctatcatactggctattatatggtaagtaatcactcagcatcttttcctgacaatttttttgtagttatgtgactttgttttgtaaatttataaaatactacttgcttctctctttatattactaaaaaataaaaataaaaaaatacaactgtctgaggcttaaattactcttgcattgtccctaagtataattttagttaattttaaaaagctttcatgctattgttagattattttgattatacacttttgaattgaaattatactttttctaaataatgttttaatctctgatttgaaattgattgtagggaatggaaaagatgggataatttttcataaatgaaaaatgaaattcttttttttttttttttttttttgagacggagtcttgctctgttgcccaggctggagtgcaatggcgtgatcttggctcacagcaagctctgcctcctggattcacgccattctcctgcctcagcctcagaggtagctgggactacaggtgcctgccaccacgcctgtctaattttttgtatttttttgtaaagacagggtttcactgtgttagccaggatggtctcaatctcctgaccccgtgatccacccgcctcggccttccaagagaaatgaaatttttttaatgcacaaagatctggggtaatgtgtaccacattgaaccttggggagtatggcttcaaacttgtcactttatacgttagtctcctacggacatgttctattgtattttagtcagaacatttaaaattattttattttattttattttttttttttttttgagacggagtctcgctctgtcacccaggctggagtacagtggcgcagtctcggctcactgcaagctccgcctcccgggttcacgccattctcctgcctcagcctctccgagtagctgggactacaggcgcccgccaccacgcccggctaatttttttttatttttagtagagacggggtttcaccgtggtctcgatctcctgacctcgtgatccacccgcctcggcctcccaaagtgctgggattacaagcgtgagccaccgcgcccggcctaaaattatttttaaaagtaagctcttgtgccctgctaaaattatgatgtgatattgtaggcacttgtatttttagtaaattaatatagaagaaacaactgacttaaaggtgtatgtttttaaatgtatcatctgtgtgtgcccccattaatattcttatttaaaagttaaggccagacatggtggcttacaactgtaatcccaacagtttgtgaggccgaggcaggcagatcacttgaggtcaggagtttgagaccagcctggccaacatgatgaaaccttgtctctactaaaaataccaaaaaaaatttagccaggcatggtggcacatgcctgtaatccgagctacttgggaggctgtggcaggaaaattgctttaatctgggaggcagaggttgcagtgagttgagattgtgccactgcactccacccttggtgacagagtgagattccatctcaaaaaaagaaaaaggcctggcacggtggctcacacctataatcccagtactttgggaggtagaggcaggtggatcacttgaggttaggagttcaggaccagcctggccaacatggtgactactccatttctactaaatacacaaaacttagcccagtggcgggcagttgtaatcccagctacttgagaggttgaggcaggagaatcacttgaacctgggaggcagaggttgcagtgagccgagatcacaccgctgcactctagcctggccaacagagtgagaatttgcggagggaaaaaaaagtcacgcttcagttgttgtagtataaccttggtatattgtatgtatcatgaattcctcattttaatgaccaaaaagtaataaatcaacagcttgtaatttgttttgagatcagttatctgactgtaacactgtaggcttttgtgttttttaaattatgaaatatttgaaaaaaatacataatgtatatataaagtattggtataatttatgttctaaataactttcttgagaaataattcacatggtgtgcagtttacctttgaaagtatacaagttggctgggcacaatggctcacgcctgtaatcccagcactttgggaggccagggcaggtggatcacgaggtcaggagatcgagaccatcctggctaacatggtgaaaccccgtctctactaaaagtacaaaaacaaattagccgggcatgttggcgggcaccttttgtcccagctgctcgggaggctgaggcaggagagtggcgtgaacccaggaggtggagcttgcagtgagccgagattgtgccagtgcactccagcctgggcgacagagcgagactctgtctcaaaaaataaaataaaaaagaaagtatacaagtcagtggttttggttttcagttatgcaaccatcactacaatttaagaacattttcatcaccccaaaaagaaaccctgttaccttcattttccccagccctaggcagtcagtacactttctgtctctatgaatttgtctattttagatattatatataaacggaattatacgatatgtggtcttttgtgtctggcttctttcacttagcatgctattttcaagattcatccatgctgtagaatgcaccagtactgcattccttcttattgctgaatattctgttgtttggttatatcacattttatccattcatcagttcatggacatttaggttgtttttatttttgggctataatgaataatgttgctatgaacattcgtttgtgttctttttgtttttttggttttttgggttttttttgttttgtttttgtttttgagacagtcttgctctgtctcctaagctggagtgcagtggcatgatcttggcttactgcaagctctgcctcccgggttcacaccattctcctgcctcagcccgacaagtagctgggactacaggcgtgtgccaccatgcacggctaattttttgtatttttagtagagatggggtttcaccgtgttagccaggatggtctcgatctcctgacctcgtgatctgcctgcctaggcctcccaaagtgctgggattacaggcgtgagccactgcacctggccttaagtgtttttaatacgtcattgccttaagctaacaattcttaacctttgttctactgaagccacgtggttgagataggctctgagtctagcttttaacctctatctttttgtcttagaaatctaagcagaatgcaaatgactaagaataatgttgttgaaataacataaaataggttataactttgatactcattagtaacaaatctttcaatacatcttacggtctgttaggtgtagattagtaatgaagtgggaagccactgcaagctagtatacatgtagggaaagatagaaagcattgaagccagaagagagacagaggacatttgggctagatctgacaagaaaaacaaatgttttagtattaatttttgactttaaattttttttttatttagtgaatactggtgtttaatggtctcattttaataagtatgacacaggtagtttaaggtcatatattttatttgatgaaaataaggtataggccgggcacggtggctcacacctgtaatcccagcactttgggaggccgaggcaggcggatcacctgaggtcgggagttagagactagcctcaacatggagaaaccccgtctctactaaaaaaaatacaaaattaggcgggcgtggtggtgcatgcctgtaatcccagctactcaggaggctgaggcaggagaattgcttgaacctgggaggtggaggttgcggtgagccgagatcacctcattgcactccagcctgggcaacaagagcaaaactccatctcaaaaaaaaaaaaataaggtataagcgggctcaggaacatcattggacatactgaaagaagaaaaatcagctgggcgcagtggctcacgccggtaatcccaacactttgggaggccaaggcaggcgaatcacctgaagtcgggagttccagatcagcctgaccaacatggagaaaccctgtctctactaaaaatacaaaactagccgggcatggtggcgcatgcctgtaatcccagctacttgggaggctgaggcaggagaattgcttgaaccgagaaggcggaggttgcggtgagccaagattgcaccattgcactccagcctgggcaacaagagcgaaactccgtctcaaaaaaaaaaggaagaaaaatatttttttaaattaattagtttatttattttttaagatggagttttgccctgtcacccaggctggggtgcaatggtgcaatctcggctcactgcaacctccgcctcctgggttcaagtgattctcctgcctcagcttcccgagtagctgtgattacagccatatgccaccacgcccagccagttttgtgttttgttttgttttttgttttttttttttgagagggtgtcttgctctgtcccccaagctggagtgcagcggcgcgatcttggctcactgcaagctctgcctcccaggttcacaccattctcttgcctcagcctcccgagtagctgggactacaggtgcccgccaccacacccggctaatttttttgtgtttttagtagagatggggtttcactgtgttagccaggatggtctcgatctcctgaccttttgatccacccgcctcagcctccccaagtgctgggattataggcgtgagccactgtgcccggcctagtcttgtatttttagtagagtcgggatttctccatgttggtcaggctgttctccaaatccgacctcaggtgatccgcccgccttggcctccaaaagtgcaaggcaaggcattacaggcatgagccactgtgaccggcaatgtttttaaattttttacatttaaattttattttttagagaccaggtctcactctattgctcaggctggagtgcaagggcacattcacagctcactgcagccttgacctccagggctcaagcagtcctctcacctcagtttcccgagtagctgggactacagtgataatgccactgcacctggctaatttttatttttatttatttatttttttttgagacagagtcttgctctgtcacccaggctggagtgcagtggtgtaaatctcagctcactgcagcctccgcctcctgggttcaagtgattctcctgcctcaacctcccaagtagctgggattagaggtccccaccaccatgcctggctaattttttgtactttcagtagaaacggggttttgccatgttggccaggctgttctcgaactcctgagctcaggtgatccaactgtctcggcctcccaaagtgctgggattacaggcgtgagccactgtgcctagcctgagccaccacgccggcctaatttttaaattttttgtagagacagggtctcattatgttgcccagggtggtgtcaagctccaggtctcaagtgatccccctacctccgcctcccaaagttgtgggattgtaggcatgagccactgcaagaaaaccttaactgcagcctaataattgttttctttgggataacttttaaagtacattaaaagactatcaacttaatttctgatcatattttgttgaataaaataagtaaaatgtcttgtgaaacaaaatgctttttaacatccatataaagctatctatatatagctatctatatctatatagctattttttttaacttcctttattttccttacagggttttagacaaaatcaaaaagaaggaaggtgctcacattccttaaattaaggagtaagtctgccagcattatgaaagtgaatcttacttttgtaaaactttatggtttgtggaaaacaaatgtttttgaacatttaaaaagttcagatgttagaaagttgaaaggttaatgtaaaacaatcaatattaaagaattttgatgccaaaactattagataaaaggttaatctacatccctactagaattctcatacttaactggttggttgtgtggaagaaacatactttcacaataaagagctttaggatatgatgccattttatatcactagtaggcagaccagcagacttttttttattgtgatatgggataacctaggcatactgcactgtacactctgacatatgaagtgctctagtcaagtttaactggtgtccacagaggacatggtttaactggaattcgtcaagcctctggttctaatttctcatttgcaggaaatgctggcatagagcagcactaaatgacaccactaaagaaacgatcagacagatctggaatgtgaagcgttatagaagataactggcctcatttcttcaaaatatcaagtgttgggaaagaaaaaaggaagtggaatgggtaactcttcttgattaaaagttatgtaataaccaaatgcaatgtgaaatattttactggactctattttgaaaaaccatctgtaaaagactgaggtgggggtgggaggccagcacggtggtgaggcagttgagaaaatttgaatgtggattagattttgaatgatattggataattattggtaattttatgagctgtgagaagggtgttgtagtttataaaagactgtcttaatttgcatacttaagcatttaggaatgaagtgttagagtgtcttaaaatgtttcaaatggtttaacaaaatgtatgtgaggcgtatgtggcaaaatgttacagaatctaactggtggacatggctgttcattgtactgtttttttctatcttctatatgtttaaaagtatataataaaaatatttaatttttttttaaa

SEQ ID NO: 81 - protein, 10 aa, Artificial sequence
  Synthetic peptide
  MISC_FEATURE (1)..(1): Xaa is selected from lysine, cysteine, D-lysine, L-lysine, N6-(2-azido-acetyl)-lysine, N6-(2-azido- acetyl)-D-lysine, and N6-(2-azido-acetyl)-L-lysine
XPAPSGPSPG

SEQ ID NO: 82 - protein, 15 aa, Artificial sequence
  Synthetic peptide
  MISC_FEATURE (1)..(1): Xaa is selected from lysine, cysteine, D-lysine, L-lysine, N6-(2-azido-acetyl)-lysine, N6-(2-azido- acetyl)-D-lysine, and N6-(2-azido-acetyl)-L-lysine
XAGSIKPPPAGSSPG

SEQ ID NO: 83 - protein, 10 aa, Artificial sequence
  Synthetic peptide
  MISC_FEATURE (1)..(1): Xaa is selected from lysine, cysteine, D-lysine, L-lysine, N6-(2-azido-acetyl)-lysine, N6-(2-azido- acetyl)-D-lysine, and N6-(2-azido-acetyl)-L-lysine
XAGMSGASAG